(19) United States
(12) Patent Application Publication
(10) Pub. No.: US 2016/0024083 A1
(43) Pub. Date: Jan. 28, 2016

(54) COMPOUNDS AND METHODS FOR TREATING CANCERS

(71) Applicant: HEALTH RESEARCH, INC., Buffalo,, NY (US)

(72) Inventors: Katerina GUROVA, Orchard Park, NY (US); Warren WADE, San Diego, CA (US)

(73) Assignee: HEALTH RESEARCH, INC., Buffalo, NY (US)

(21) Appl. No.: 14/775,732

(22) PCT Filed: Mar. 14, 2014
(86) PCT No.: PCT/US14/28813

§ 371 Date: Sep. 14, 2015

Related U.S. Application Data
(60) Provisional application No. 61/781,334, filed on Mar. 14, 2013.

(51) Int. Cl. C07D 471/04; C07D 209/86; C07D 409/04; C07D 403/04; C07D 413/04; C07D 401/04; C07D 417/04; C07D 209/88; C07D 405/04

(52) U.S. Cl. CPC C07D 471/04; C07D 209/88; C07D 209/86; C07D 409/04; C07D 405/04; C07D 413/04; C07D 401/04; C07D 417/04; C07D 403/04

(57) ABSTRACT

Provided are carbazole and carbazole-like compounds (e.g., pyridoindole and pyrrolodipyridine) compounds, that can be used to selectively kill cancer cells, specifically androgen-receptor expressing prostate cancer cells. Also provided is a method of treating AR-positive prostate cancer in a subject diagnosed with or suspected of having AR positive or negative cancer, comprising administering an effective amount of a carbazole and carbazole-like compound to said subject.

### CROSS-REFERENCE TO RELATED APPLICATIONS

This application claims priority to U.S. provisional patent application No. 61/781,334, filed Mar. 14, 2013, the disclosure of which is incorporated herein by reference.

### FIELD OF THE DISCLOSURE

The present disclosure generally relates to carbazole and carbazole-like compounds and methods of making and using such compounds.

### BACKGROUND OF THE DISCLOSURE

Prostate cancer (PCa) is the most frequent neoplastic disease and the second leading cause of cancer-related deaths in men, claiming more than 30,000 men each year in the United States alone. PCa tumors are composed primarily of prostate luminal epithelial cells. Differentiation of prostate luminal epithelial cells is controlled in part by Androgen receptor (AR) driven expression of prostate-specific markers. AR controls survival of the cells through mechanisms that remain unclear. In addition to prostate cancer, AR is in involved in the etiology of other cancers, including breast cancers. AR belongs to the family of steroid receptors and functions as a transcription factor. In the absence of ligand, members of this family are unstable proteins that reside in the cytoplasm bound to Heat Shock Protein 90 (Hsp90). Upon binding of a steroid such as androgen to the ligand binding domain (LBD) of AR, AR is freed from Hsp90 and translocates to the nucleus. Androgen-bound AR in the nucleus activates transcription of genes with androgen responsive elements (ARE) in their promoters (Cato, A. C., et al. 1998. Trends Endocrinol Metab 9: 150-154). In addition to its function as a transcriptional activator, AR is also capable of repressing transcription of some genes (Claessens, et al. 2001. J Steroid Biochem Mol Biol 76:23-30).

Depletion of androgens causes death of normal prostate luminal epithelial cells, which demonstrates the critical role of the AR pathway in their survival. Cancerous prostate cells continue to express AR and their survival also depends on the presence of androgens, which makes androgen deprivation the therapy of choice for patients with advanced PCa. Anti-androgen therapies, including use of the inhibitors flutamide and casodex, are usually effective initially, but rarely result in a complete cure. PCa relapse occurs in most of patients treated with such therapies, which leads to androgen-independent, chemotherapy-resistant tumors with poor prognosis. Thus, resistance to anti-androgen therapy is a major obstacle in successful treatment of PCa.

Analysis of the mechanisms of androgen-independence acquired by PCa during tumor progression indicates that loss of AR signalling is involved rarely (Balk, S. P. 2002. Urology 60: 132-138; discussion 138-139). On the contrary, androgen-independent PCa is typically characterized by heightened AR activity due to expression of AR mutants that are ligand-independent (constitutively active) or responsive to non-androgen ligands (Chen, Y., et al. 2008. Curr Opin Pharmacol 8:440-448; Tilley, et al. 1996. Clin Cancer Res 2:277-285; Koivisto, et al. 1998 Am J Pathol 152: 1-9; Marques, et al. 2005. Int J Cancer 117:221-229; Bohl, C et al. 2005. J Biol Chem 280:37747-37754; Hara, T., et al. 2003. Cancer Res 63: 149-153).

It was recently shown that unlike normal prostate stem cells, prostate “tumor initiating cells” or “cancer stem cells”, a minor cell population believed to be the major source of self-renewing tumor cells, express functional AR (Vander Griend, et al. 2008. Cancer Res 68:9703-97111). This, together with the observed maintenance of AR activity in PCa tumors that have progressed to the stage of castration resistance, indicates that AR is a promising potential therapeutic target for both androgen-dependent and -independent PCa, as well as other AR positive cancer types. For example, breast epithelial cells are, in many regards, similar to prostate cells. As the survival of PC cells depend upon the androgen-stimulated activity AR, breast epithelial cells are similarly dependent upon the related estrogen (ER) and progesterone receptors (PR). The role of ER and PR in breast cancer (BC) and modulation of their function as a therapeutic approach has been the focus of studies for many years.

However, AR is expressed at low levels in normal mammary cells and at different levels in a majority of BCs, including 50% of “triple negative” (ER-, PR-, Her2-) BCs, for which targeted therapy is not yet available. Although the effect of androgens on breast epithelial cells has been addressed in several studies, the role played by AR in BC remains unclear (Birrell, et al. (1995) J Steroid Biochem Mol Biol 52, 459-467; Brettes, et al. (2008) Bull Cancer 95, 495-502; Di Monaco, et al. (1995) Anticancer Res 15, 2581-2584). Thus, current treatment modalities are largely ineffective for AR positive cancers, and there is an ongoing need for new methods for therapy of AR positive cancer cells, including but not limited to PCa and breast cancer.

### BRIEF SUMMARY OF THE DISCLOSURE

In an aspect, the present disclosure provides heterocyclic compounds having the following structure:

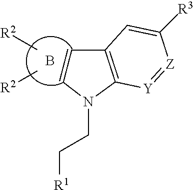

where R¹ is selected from the group consisting of H, CH₃, CH₂F, CHF₂, and CF₃; Y and Z each independently are a nitrogen atom or carbon atom; ring B is a unsubstituted or substituted aryl or heteroaryl ring; each R² is independently a hydrogen atom, a halogen atom, an alkoxy group, a nitrile group, or an amide group, and R³ is a five membered or six membered unsubstituted or substituted heterocycle, ketone, or nitrile. In certain embodiments, R³ is a five or six membered heteroaryl ring. The compound has 0-2 R² groups.

In an embodiment, the present disclosure provides heterocyclic compounds having the following structure:

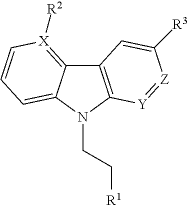

where R¹ is selected from the group consisting of H, CH₃, CH₂F, CHF₂, and CF₃. X, Y and Z each independently are a nitrogen atom or carbon atom. When X is a carbon atom then R² is a hydrogen atom, a halogen atom, an alkoxy group, a nitrile group, or an amide group. When X is a nitrogen atom, R² is absent. R³ is a ketone, nitrile, or a five membered or six membered unsubstituted or substituted heterocycle. In certain embodiments, R³ is a five or six membered heteroaryl ring.

In an embodiment, R¹ is not a hydrogen atom. In an embodiment, R¹ is not CH₃. In an embodiment, each R² is independently not a hydrogen atom. In an embodiment, each R² is independently not an alkoxy group. In an embodiment, R³ is not a ketone. In another embodiment, the compounds of the present disclosure are as described herein, with the proviso they do not have the following structure:

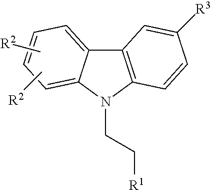

where R¹ is a hydrogen atom or CH₃, each R² is independently a hydrogen atom, an alkoxy group, a ketone, or a halide group and R³ is a ketone group.

In an embodiment, in the following structure R² is not the same as R³

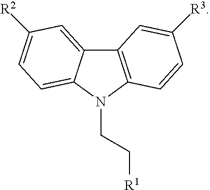

In an embodiment, in the following structure R² is a hydrogen atom and R¹ and R³ are as defined herein:

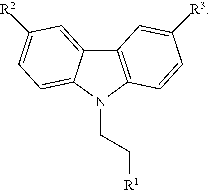

In an embodiment, the compound has the following structure:

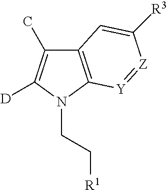

where C and D are replaced by the atoms of the following structures to form a ring:

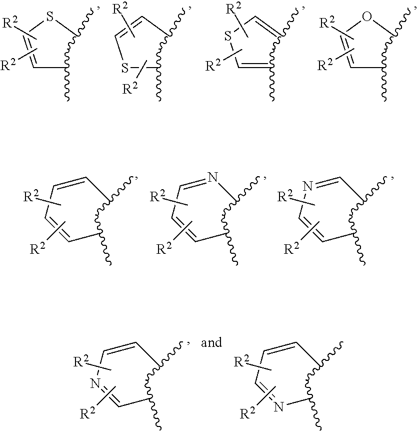

which can be optionally substituted with 0, 1, or 2 R² groups and R¹, R², R³, Y, and Z are as defined herein. In certain embodiments, the double bond between C and D is a single bond. For example, when C and D are replaced by

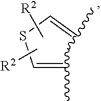

the bond between C and D is a single bond.

In an embodiment, R³ is a heterocyclic ring that is aromatic. In another embodiment, R³ is a heterocyclic ring that is partially unsaturated or unsaturated.

In an embodiment, R³ is selected from one of the following structures:

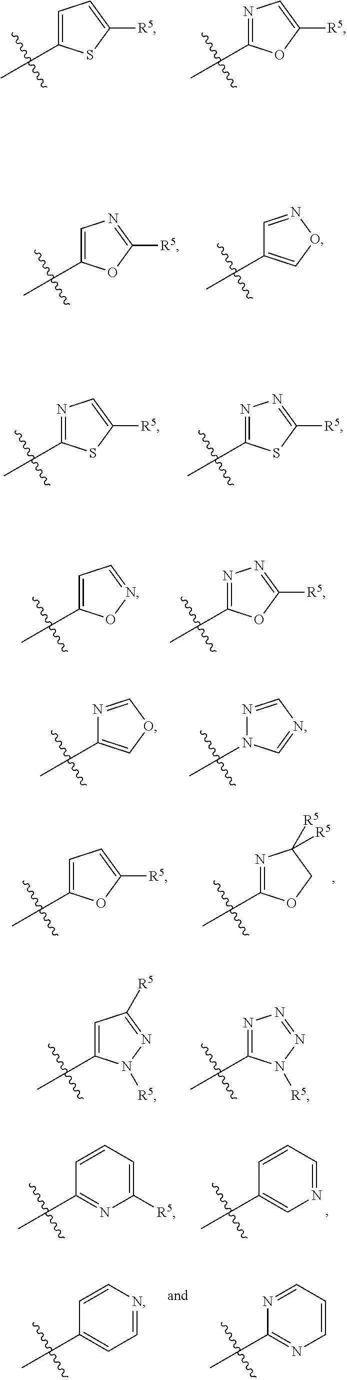

where each R⁵ is independently a hydrogen atom or alkyl group. In another embodiment, R³ is

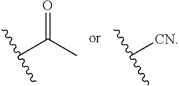

In an embodiment, the compound has the following structure:

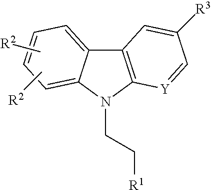

where R¹, R², R³, and Y are as defined herein.

In an embodiment, the compound has the following structure:

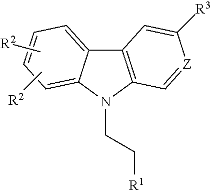

where R¹, R², R³, and Z are as defined herein.

In an embodiment, the compound has the following structure:

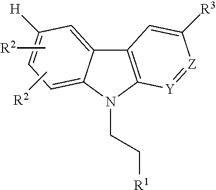

where R¹, R², R³, Y, and Z are as defined herein.

In an embodiment, the compound has the following structure:

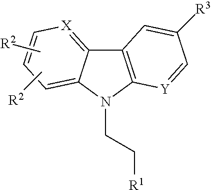

where R¹, R², R³, X, and Y are as defined herein.

In an embodiment, the compound has the following structure:

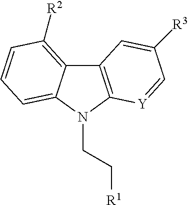

where R¹, R², Y are as defined herein and R³ is a ketone or nitrile.

In an embodiment, the compound has the following structure:

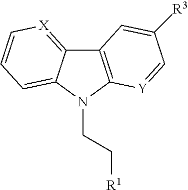

where R¹, X, and Y are as defined herein and R³ is a ketone or nitrile.

In an embodiment, the compound has the following structure:

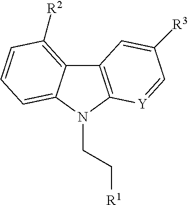

where R¹, R³, and Y are as defined herein and R² in this embodiment is a fluorine atom, an alkoxy group, a nitrile group, or an amide group.

In an embodiment, the compound has the following structure:

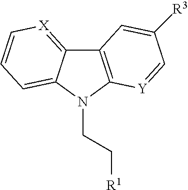

where R¹, X, and Y are as defined herein and R³ is a heterocyclic ring that only contains oxygen, sulfur, or a combination thereof.

In an embodiment, the compound has the following structure:

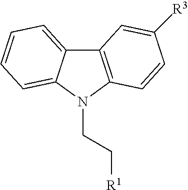

where R¹ is H, CH₃, CH₂F, CHF₂, or CF₃ and R³ is a heterocyclic ring.

In an embodiment, the compound has the following structure:

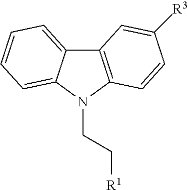

where R¹ is H, CH₃, CH₂F, CHF₂, or CF₃ and R³ is a heteroaryl ring.

In an embodiment, the compound of the present disclosure has the following structure:

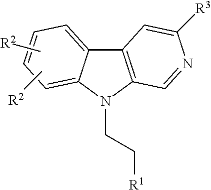

where R¹ and R² are as defined herein and R³ is a ketone or nitrile.

### BRIEF DESCRIPTION OF THE FIGURES

FIGS. 1A-F. Cytotoxicity data for examples of the compounds.

FIG. 2. Metabolic stability of examples of the compounds in the presence of mouse hepatocytes. a) Half-life was calculated as t_{1/2}=0.693/k, where k is the elimination rate constant in the equation describing first order decay (Ct=C0*e^{-kt}), and Cₜ and C₀ are the peak area ratios at time t and time 0, respectively. Data points were fitted to a first-order decay model by non-linear regression, using GraphPad Prism (version 5.04 or higher) without weighting or any user intervention. When the percent remaining was >50% at the longest incubation time or <50% at the shortest incubation time, the half-life is expressed as >the longest incubation time or <the shortest incubation time, respectively, and the calculated half-life is given in parentheses. b) Intrinsic clearance (C_{Lint}) was calculated based on C_{Lint}=k/P, where k is the elimination rate constant and P is the protein concentration in the incubation. NF-peak not found

FIG. 3. Comparison of microsomal stability of examples of the compounds in the presence of mouse hepatocytes (HS) and mouse liver microsomes (MS).

FIG. 4. Pharmacokinetic profiles for examples of the compounds. The table shows concentrations of the compounds (μg) per ml of plasma at different time points after administration of the indicated dose IP or IV. Below is a graphical view of the same data.

FIG. 5. Dynamic of weight of mice treated with 5 daily IV or IP doses of PLA1125 or PLA1079.

FIG. 6. Growth of CWR22R tumors in SCID mice treated with 5 daily IV or IP doses of vehicle or PLA1125 or PLA1079. Data shown as fold of increase of tumor volume comparing with the day of start of treatment.

FIG. 7. Concentrations of PLA1079 and PLA1125 in tumors of mice treated with 5 daily doses of the drugs. Tumors were collected 24 hours after last administration.

FIG. 8. Pharmacokinetic (PK) data for PLA1098.

FIG. 9. PK data of PLA1148. Data from the mouse injected twice are shown in red.

FIG. 10. Scheme and summary of PLA1098 pilot efficacy testing. On the top is the scheme of drug administration and samples collection. First mouse was euthanized on day 3 and drug concentration was measured in plasma, liver and two tumors. Data are shown near first red arrow. Other 4 mice were euthanized on day 12. Plots demonstrate curves of individual tumor growth in control and PLA1098 group mice. Bar diagram shows average weight of excised tumors from control and PLA1098 treated mice+/−standard deviation.

FIG. 11. Expression of Caveolin 1 gene in a panel of breast cancer cell lines with different c52 sensitivity.

FIG. 12. Expression of Caveolinl in sensitive versus resistant cells. Caveolinl is expressed in resistant MDA MB 231 cells, but not in sensitive MCF7 cells. Treatment of cells with c52 or PLA1079 did not change levels of Caveolinl expression. Gapdh was used as a loading control, overexpression of p21 in sensitive cells upon treatment with the compounds confirmed the activity of used compounds.

FIG. 13. c52 causes a DNA-damage-response type of p53 activation in sensitive, but not resistant cells. Results of Western Blot analysis. Sensitive (MCF7 and CWR22r) and resistant (NKE) cells were treated with 1 uM of c52 for indicated time-periods. In sensitive, but not resistant cells c52 caused elevation of p53 amount, more than that c52 treatment induced phosphorylation of p53 by Serines 15 and 329—hallmarks of DNA damage response activation.

FIG. 14. c52 causes replication stress in sensitive, but not resistant cells. A. Immuno-histological staining with specific antibodies to RPA70 and XRCC1 (proteins, accumulating at sites of stalled replication forks) in sensitive (MCF7) or resistant (MDA MB 231) cells treated with c52 versus control untreated ones. Clear formation of loci of both proteins might be observed in treated sensitive cells. B. c52 treatment abrogates incorporation of Edu in sensitive MCF7 cells, indicating stall of replication.

FIG. 15. c52 induces accumulation of Mdm2 in sensitive CWR22r cells. Results of Western Blot analysis: CWR22r cells were treated with 1 uM c52 for indicated time intervals.

FIG. 16. Inhibition of p53 activity by expression of its dominant-negative forms does not abrogate c52-caused degradation of AR level in CWR22r or MCF7 cells. A. Though p53 activity is inhibited by introduction of its dominant-negative mutant we still observe decrease of AR upon c52 treatment in CWR22r cells. B. In MCF7 cells with functionally inactive p53 (R175H, GSE56) Mdm2 is no longer overexpressed upon treatment with PLA1079 or p53 activator CBL 137. Still, AR is being degraded upon this treatment.

FIG. 17. Summary of PK data of tested PLA compounds.

FIG. 18. Scheme and data of pilot efficacy testing of the compound PLA1163. Curves which end earlier than other indicate tumors which were collected to measure intra-tumor drug concentration at the end of treatment.

FIG. 19. Weight of CWR22R tumors excised from mice at the end of experiment (day 12 after start of treatment). Bars—mean of tumor weight within each group (n=5-10), error bars—standard deviation.

FIG. 20. Plot of plasma concentrations of different PLA compounds at different time points after single IV administration of 50 mg/kg.

FIG. 21. Toxicity of PLA1055 to CWR22R cells in vitro depending on time of incubation. PLA1055 was added to CWR22R in full range of concentrations for the periods of time shown on the right. After that drug containing media were changed for drug free and survival of cells was detected at 72 hours after start of treatment using Alamar Blue assay (Promega).

FIG. 22. Activation of Caspase 3/7 by c52 vs Doxorubicin (Dox) in sensitive and resistant cells with a different status of p53. Cells were incubated with c52 or doxorubicin in indicated concentrations for 16 hours. After that substrate to Caspases 3/7 was added and activity of caspases 3/7 (which would indicate apoptosis occurrence) was estimated by measuring the substrate cleavage.

FIG. 23. Ectopic Caveolinl did not save sensitive cells from sensitivity to c52. A. Results of WB analysis: AR is being decreased and p53 activated in Caveolinl expressing cells, same as in regular ones. B. MCF7 cells, introduced with Caveolinl expressing construct, remain sensitive to c52.

FIG. 24. DARTS (Drug Affinity Responsive Target Stability) assay was performed using c52 and PLA1118. According to this assay these compounds are capable of protecting presumable target protein from protease degradation. Protein lysates from sensitive CWR22r cells were incubated with or without the drug and subsequently digested with the indicated concentration of pronase. A. c52 was used (lane T-treated, M-marker) with indicated concentrations of pronase; ability of c52 to protect its target from protease cleavage was judged based on presence of protein band in the treated lane vs the untreated (UT) control. B. Additional compounds PLA1098 (an active analogue of c52) and PLAl 118 (inactive analog) were used to confirm the results. The protein band appeared in c52 and PLA1098 (shown by arrows) as opposed to the untreated and faintly in PLA1118 samples indicating that both of the active compounds protected their targets from degradation.

FIG. 25. Efficacy (A) and Stability (B) of biotinylated PLA1200 and PLA1201 compounds.

### DETAILED DESCRIPTION OF THE DISCLOSURE

The present disclosure provides carbazole compounds and carbazole-like compounds (e.g., pyridoindole and pyrrolodipyridine compounds). The compounds can selectively kill cancer cells.

In an aspect, the present disclosure provides heterocyclic compounds having the following structure:

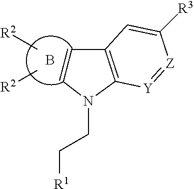

where R¹ is selected from the group consisting of H, CH₃, CH₂F, CHF₂, and CF₃; Y and Z each independently are a nitrogen atom or carbon atom; ring B is a unsubstituted or substituted aryl or heteroaryl ring; each R² is independently a hydrogen atom, a halogen atom, an alkoxy group, a nitrile group, or an amide group, and R³ is a five membered or six membered unsubstituted or substituted heterocycle, ketone, or nitrile. In certain embodiments, R³ is a five or six membered heteroaryl ring. The compound has 0-2 R² groups.

In an embodiment, the present disclosure provides heterocyclic compounds having the following structure:

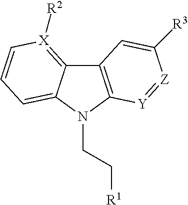

where R¹ is selected from the group consisting of H, CH₃, CH₂F, CHF₂, and CF₃. X, Y and Z each independently are a nitrogen atom or carbon atom. When X is a carbon atom then R² is a hydrogen atom, a halogen atom, an alkoxy group, a nitrile group, or an amide group. When X is a nitrogen atom, R² is absent. R³ is a ketone, nitrile, or a five membered or six membered unsubstituted or substituted heterocycle. In certain embodiments, R³ is a five or six membered heteroaryl ring.

In an embodiment, R¹ is not a hydrogen atom. In an embodiment, R¹ is not CH₃. In an embodiment, each R² is independently not a hydrogen atom. In an embodiment, each R² is independently not an alkoxy group. In an embodiment, R³ is not a ketone. In another embodiment, the compounds of the present disclosure are as described herein, with the proviso they do not have the following structure:

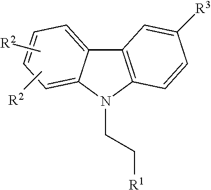

where R¹ is a hydrogen atom or CH₃, each R² is independently a hydrogen atom, an alkoxy group, a ketone, or a halide group and R³ is a ketone group.

In an embodiment, in the following structure R² is not the same as R³:

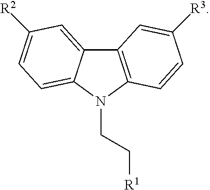

In an embodiment, in the following structure R² is a hydrogen atom and R¹ and R³ are as defined herein:

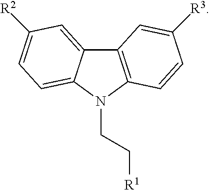

As used herein, the term “alkyl group” refers to branched or unbranched hydrocarbons. Examples of such alkyl groups include methyl groups, ethyl groups, propyl groups, butyl groups, isopropyl groups, tert-butyl groups, and the like. For example, the alkyl group can be a C₁ to C₅ alkyl group, including all integer numbers of carbons and ranges of numbers of carbons therebetween. The alkyl groups can be substituted with various other functional groups.

As used herein, the term “halogen atom” refers to a fluorine, chlorine, bromine, or iodine atom.

As used herein, the term “nitrile” refers to the following structure:

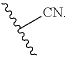 [SMILES: *C#N]

As used herein, the term “ketone” refers to the following structure:

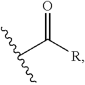

where R is an alkyl group as described herein.

As used herein, the term “amide” refers to the following structure:

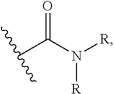

where each R is independently a hydrogen atom or alkyl group. Thus, the amide can be a primary, secondary, or tertiary amide.

As used herein, the term “aryl ring” refers to an aromatic carbocyclic group of 6 carbon atoms having a single ring (e.g., phenyl). The aryl group is substituted with 0, 1, or 2 R² groups as described herein.

As used herein, the term “heteroaryl ring” refers to an aromatic cyclic ring (i.e., fully unsaturated) having 1, 2, 3, 4, or 5 carbon atoms and 1, 2, 3, 4, or 5 heteroatoms selected from oxygen, nitrogen, sulfur, and phosphorus. Examples of heteroaryl rings include thiophene, furan, and pyridine. The heteroaryl group is substituted with 0, 1, or 2 R² groups as described herein.

As used herein, the term “heterocycle” or “heterocyclic ring” refers to a cyclic compound having a ring where at least one or more of the atoms forming the ring is a heteroatom (e.g., oxygen, nitrogen, sulfur, phosphorus). The heterocyclic ring can be aromatic or nonaromatic, and include compounds that are saturated, partially unsaturated, and fully unsaturated. Examples of such groups include furan, thiophene, oxazole, isoxazole, thiazole, oxadiazole, thiadiazole, triazole, tetrazole, dihydrooxazole, lactam, lactone, furanone, oxazolone, pyridinone, pyrimidinone, dihydropyridazine, pyranone, oxazinone, and the like. For example, the heterocyclic ring can be a C₅ to C₇ ring, including all integer numbers of carbons and ranges of numbers of carbons therebetween. The heterocyclic ring can be unsubstituted or substituted with groups such as, for example, alkyl groups, halogen atoms, amides, and nitriles

As used herein, the term “carbocyclic ring” refers to a cyclic compound having a ring in which all of the atoms forming the ring are carbon atoms. The carbocyclic ring can be aromatic or nonaromatic, and include compounds that are saturated and partially unsaturated, and fully unsaturated. Examples of such groups include, cyclohexanone, cyclopentanone, cyclopentanol, and the like. For example, the carbocyclic ring is a C₅ to C₇ carbocyclic ring, including all integer numbers of carbons and ranges of numbers of carbons therebetween.

In an embodiment, the compound has the following structure:

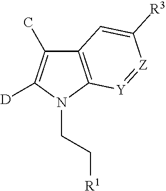

where C and D are replaced by the atoms of the following structures to form a ring:

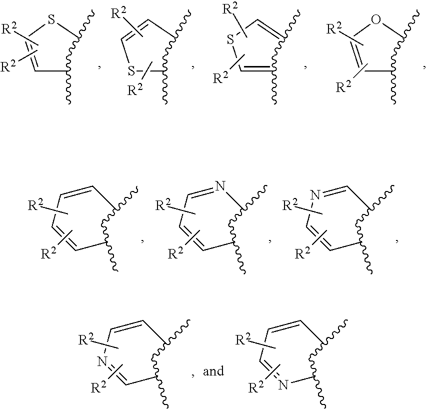

which can be optionally substituted with 0, 1, or 2 R² groups and R¹, R², R³, Y, and Z are as defined herein. In certain embodiments, the double bond between C and D is a single bond. For example, when C and D are replaced by

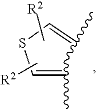

the bond between C and D is a single bond.

In an embodiment, R³ is a heterocyclic ring that is aromatic. In another embodiment, R³ is a heterocyclic ring that is partially unsaturated or unsaturated.

In an embodiment, R³ is selected from one of the following structures:

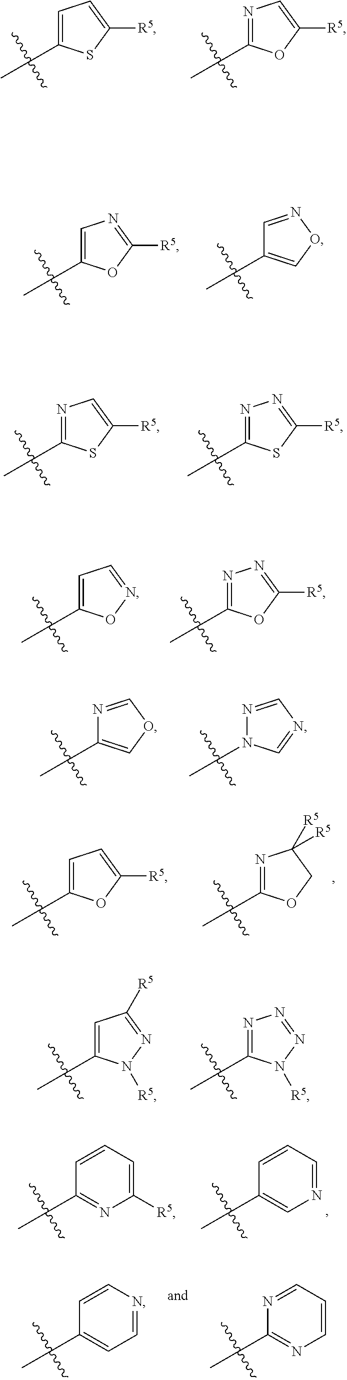

where each R⁵ is independently a hydrogen atom or alkyl group. In another embodiment, R³ is

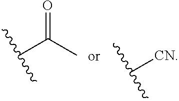

In various embodiments, the compound is a salt (e.g., a hydrochloride salt, an N-oxide), a partial salt, a hydrate, a polymorph, a stereoisomer or a mixture thereof. The compounds can have stereoisomers. For example, the compound is present as a racemic mixture, a single enantiomer, a single diastereomer, mixture of enantiomers, or mixture of diastereomers.

In an embodiment, the compound has the following structure:

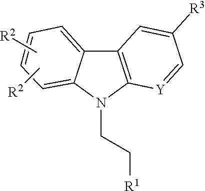

where R¹, R², R³, and Y are as defined herein.

In an embodiment, the compound has the following structure:

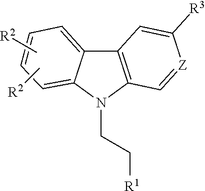

where R¹, R², R³, and Z are as defined herein.

In an embodiment, the compound has the following structure:

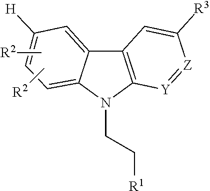

where R¹, R², R³, Y, and Z are as defined herein.

In an embodiment, the compound has the following structure:

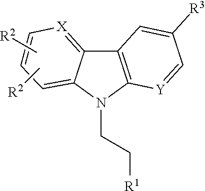

where R¹, R², R³, X, and Y are as defined herein.

In an embodiment, the compound has the following structure:

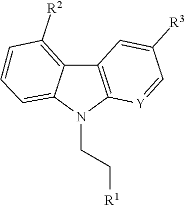

where R¹, R², Y are as defined herein and R³ is a ketone or nitrile.

In an embodiment, the compound has the following structure:

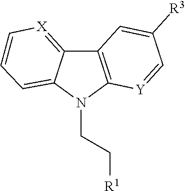

where R¹, X, and Y are as defined herein and R³ is a ketone or nitrile.

In an embodiment, the compound has the following structure:

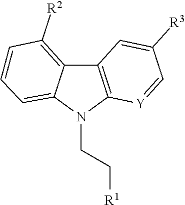

where R¹, R³, and Y are as defined herein and R² in this embodiment is a fluorine atom, an alkoxy group, a nitrile group, or an amide group.

In an embodiment, the compound has the following structure:

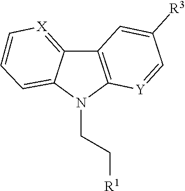

where R¹, X, and Y are as defined herein and R³ is a heterocyclic ring that only contains oxygen, sulfur, or a combination thereof.

In an embodiment, the compound has the following structure:

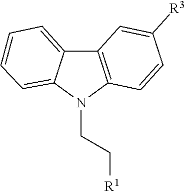

where R¹ is H, CH₃, CH₂F, CHF₂, or CF₃ and R³ is a heterocyclic ring.

In an embodiment, the compound has the following structure:

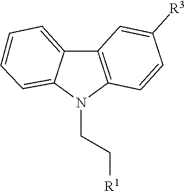

where R¹ is H, CH₃, CH₂F, CHF₂, or CF₃ and R³ is a heteroaryl ring.

In an embodiment, the compound of the present disclosure has the following structure:

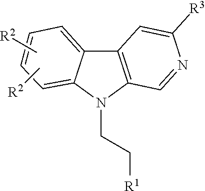

where R¹ and R² are as defined herein and R³ is a ketone or nitrile.

In an embodiment, the compound has one of the following structures:

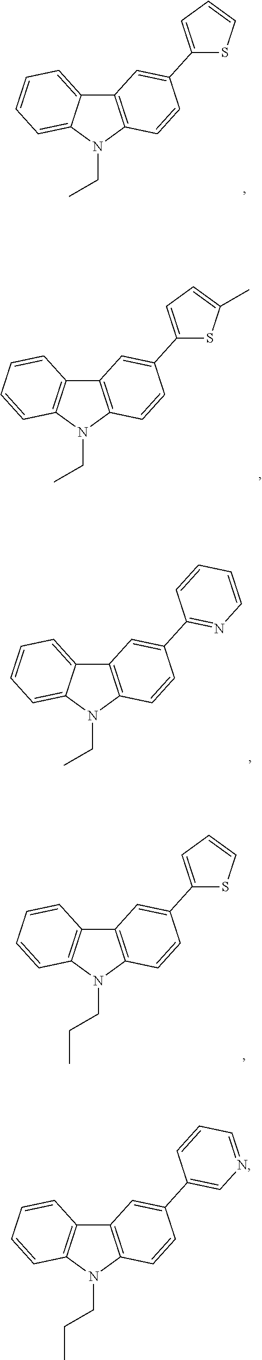 [SMILES: CCn1c2ccccc2c2cc(-c3cccs3)ccc21 ; CCn1c2ccccc2c2cc(-c3ccc(C)s3)ccc21 ; CCn1c2ccccc2c2cc(-c3ccccn3)ccc21 ; CCCn1c2ccccc2c2cc(-c3cccs3)ccc21 ; CCCn1c2ccccc2c2cc(-c3cccnc3)ccc21]

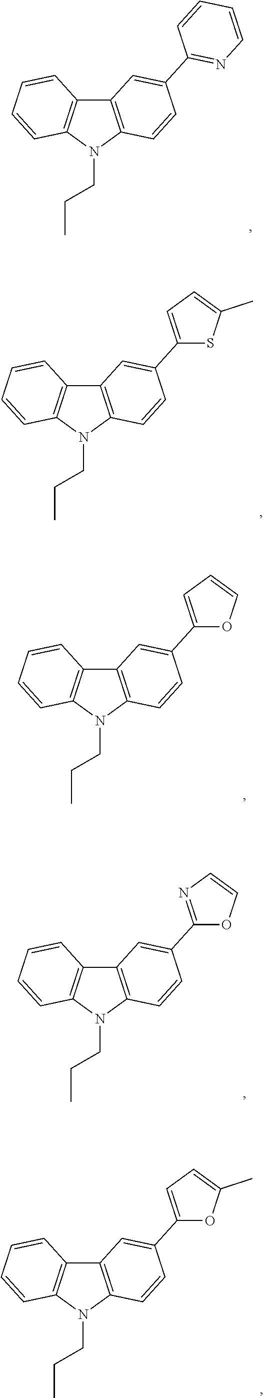 [SMILES: CCCn1c2ccccc2c2cc(-c3ccccn3)ccc21 ; CCCn1c2ccccc2c2cc(-c3ccc(C)s3)ccc21 ; CCCn1c2ccccc2c2cc(-c3ccco3)ccc21 ; CCCn1c2ccccc2c2cc(-c3ncco3)ccc21 ; CCCn1c2ccccc2c2cc(-c3ccc(C)o3)ccc21]

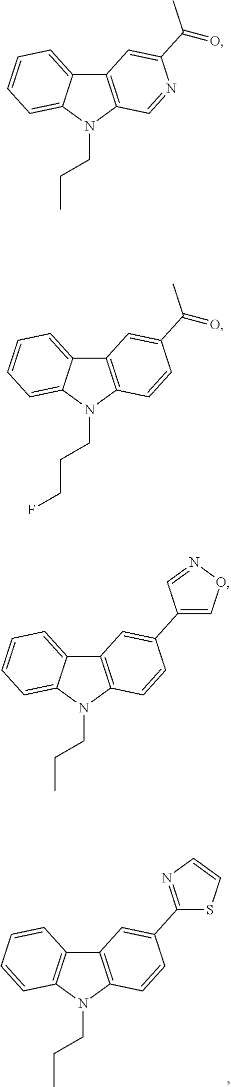 [SMILES: CCCn1c2ccccc2c2cc(C(C)=O)ncc21 ; CC(=O)c1ccc2c(c1)c1ccccc1n2CCCF ; CCCn1c2ccccc2c2cc(-c3cnoc3)ccc21 ; CCCn1c2ccccc2c2cc(-c3nccs3)ccc21]

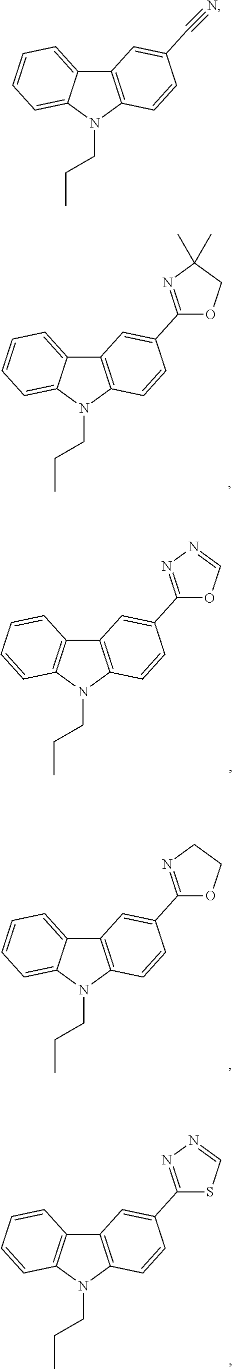 [SMILES: CCCn1c2ccccc2c2cc(C#N)ccc21 ; CCCn1c2ccccc2c2cc(C3=NC(C)(C)CO3)ccc21 ; CCCn1c2ccccc2c2cc(-c3nnco3)ccc21 ; CCCn1c2ccccc2c2cc(C3=NCCO3)ccc21 ; CCCn1c2ccccc2c2cc(-c3nncs3)ccc21]

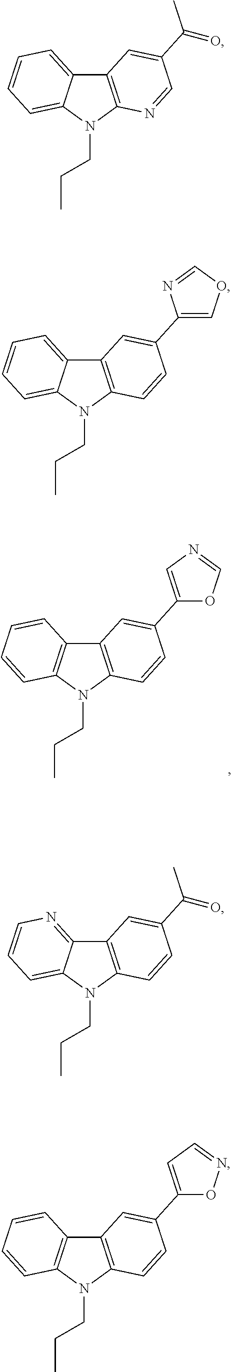 [SMILES: CCCn1c2ccccc2c2cc(C(C)=O)cnc21 ; CCCn1c2ccccc2c2cc(-c3cocn3)ccc21 ; CCCn1c2ccccc2c2cc(-c3cnco3)ccc21 ; CCCn1c2ccc(C(C)=O)cc2c2ncccc21 ; CCCn1c2ccccc2c2cc(-c3ccno3)ccc21]

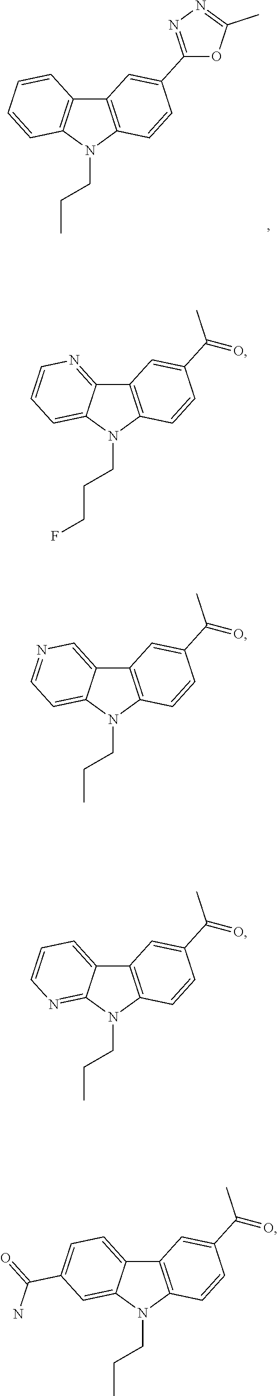 [SMILES: CCCn1c2ccccc2c2cc(-c3nnc(C)o3)ccc21 ; CC(=O)c1ccc2c(c1)c1ncccc1n2CCCF ; CCCn1c2ccncc2c2cc(C(C)=O)ccc21 ; CCCn1c2ccc(C(C)=O)cc2c2cccnc21 ; CCCn1c2ccc(C(C)=O)cc2c2ccc(C(N)=O)cc21]

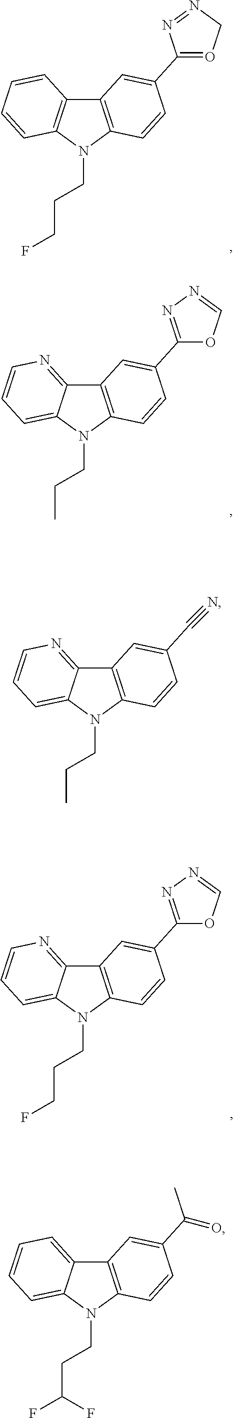

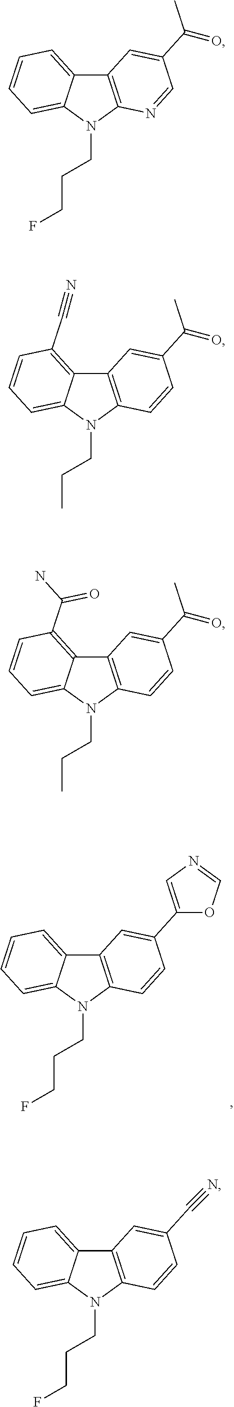 [SMILES: CC(=O)c1cnc2c(c1)c1ccccc1n2CCCF ; CCCn1c2ccc(C(C)=O)cc2c2c(C#N)cccc21 ; CCCn1c2ccc(C(C)=O)cc2c2c(C(N)=O)cccc21 ; FCCCn1c2ccccc2c2cc(-c3cnco3)ccc21 ; N#Cc1ccc2c(c1)c1ccccc1n2CCCF]

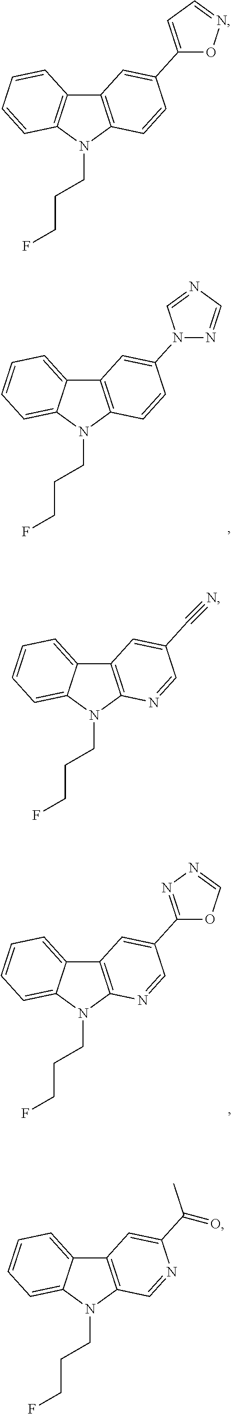 [SMILES: FCCCn1c2ccccc2c2cc(-c3ccno3)ccc21 ; FCCCn1c2ccccc2c2cc(-n3cncn3)ccc21 ; N#Cc1cnc2c(c1)c1ccccc1n2CCCF ; FCCCn1c2ccccc2c2cc(-c3nnco3)cnc21 ; CC(=O)c1cc2c3ccccc3n(CCCF)c2cn1]

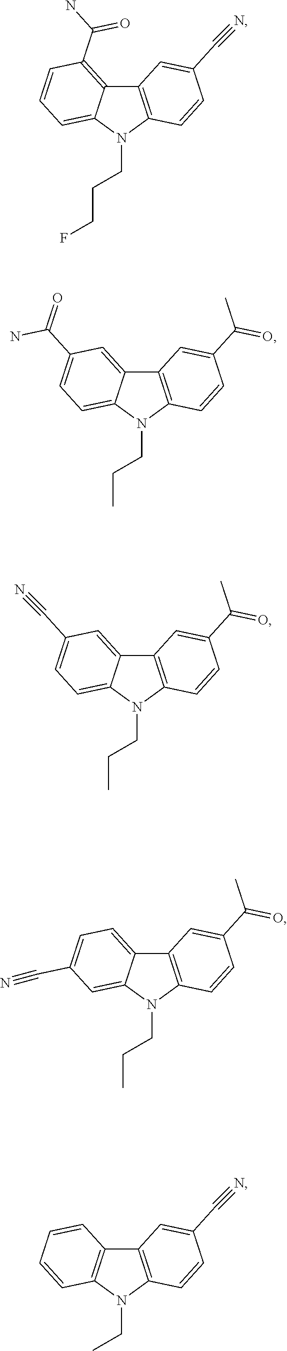 [SMILES: N#Cc1ccc2c(c1)c1c(C(N)=O)cccc1n2CCCF ; CCCn1c2ccc(C(C)=O)cc2c2cc(C(N)=O)ccc21 ; CCCn1c2ccc(C#N)cc2c2cc(C(C)=O)ccc21 ; CCCn1c2ccc(C(C)=O)cc2c2ccc(C#N)cc21 ; CCn1c2ccccc2c2cc(C#N)ccc21]

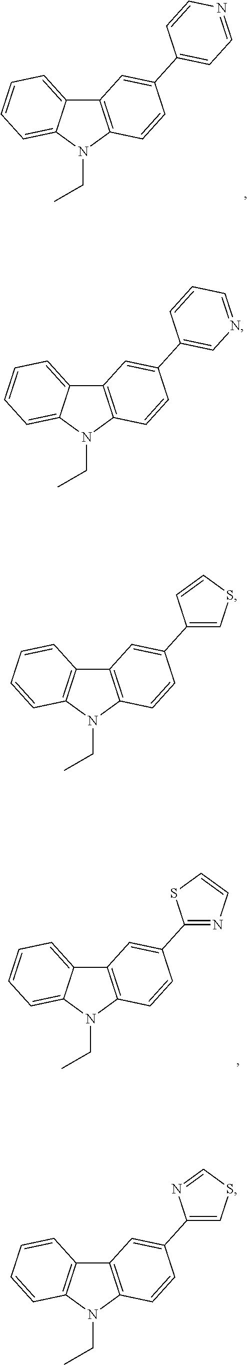 [SMILES: CCn1c2ccccc2c2cc(-c3ccncc3)ccc21 ; CCn1c2ccccc2c2cc(-c3cccnc3)ccc21 ; CCn1c2ccccc2c2cc(-c3ccsc3)ccc21 ; CCn1c2ccccc2c2cc(-c3nccs3)ccc21 ; CCn1c2ccccc2c2cc(-c3cscn3)ccc21]

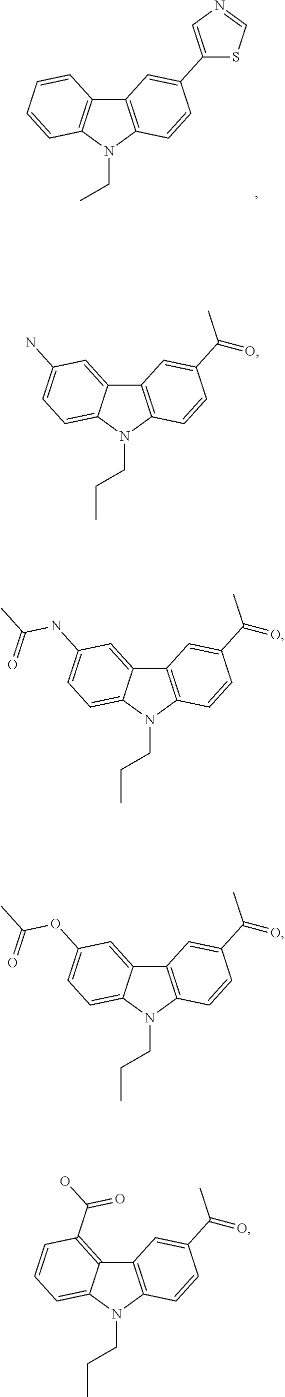 [SMILES: CCn1c2ccccc2c2cc(-c3cncs3)ccc21 ; CCCn1c2ccc(N)cc2c2cc(C(C)=O)ccc21 ; CCCn1c2ccc(NC(C)=O)cc2c2cc(C(C)=O)ccc21 ; CCCn1c2ccc(OC(C)=O)cc2c2cc(C(C)=O)ccc21 ; CCCn1c2ccc(C(C)=O)cc2c2c(C(=O)O)cccc21]

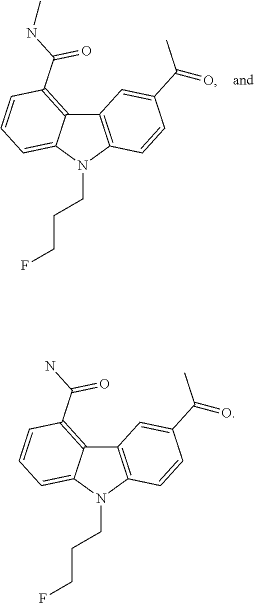 [SMILES: CNC(=O)c1cccc2c1c1cc(C(C)=O)ccc1n2CCCF ; CC(=O)c1ccc2c(c1)c1c(C(N)=O)cccc1n2CCCF]

The compounds of the present disclosure are as described herein, with the proviso that the compounds are not those described in WO/2011/050353 (also PCT/US/2010/053916 or US 2012/0264771).

Non-limiting examples of general methods for the preparation of the compounds of the present disclosure are provided in the following schemes:

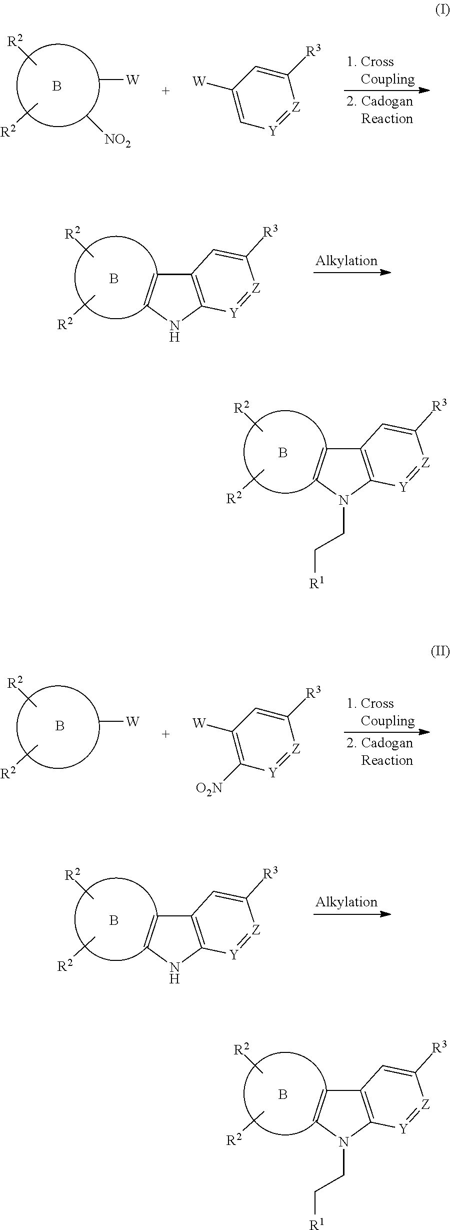

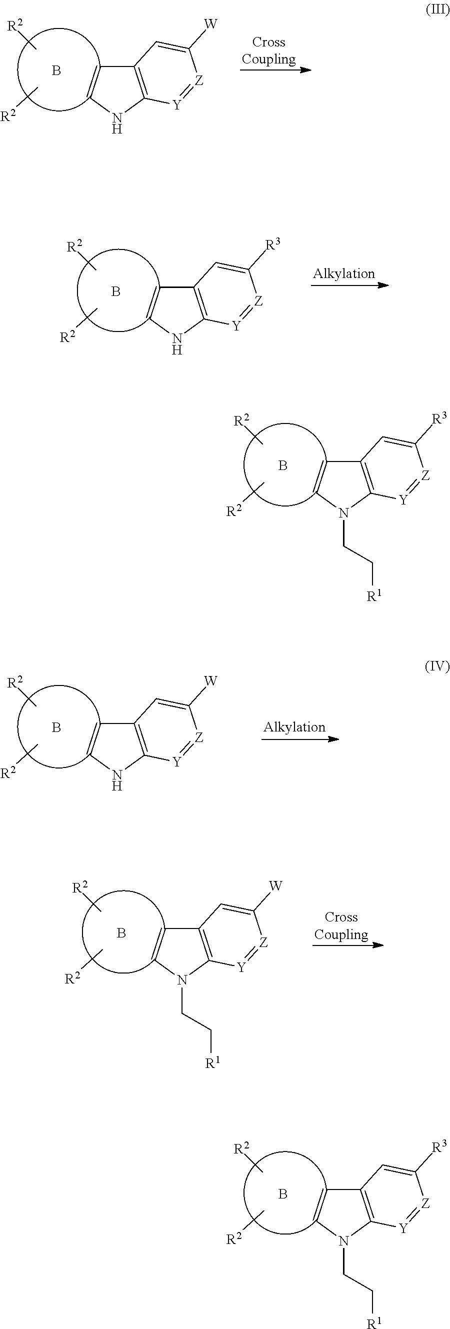

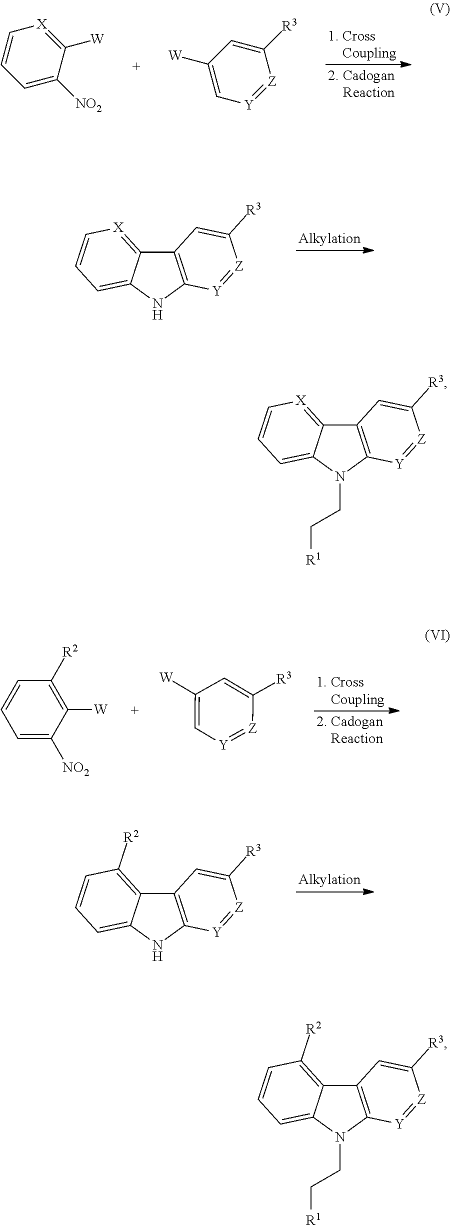

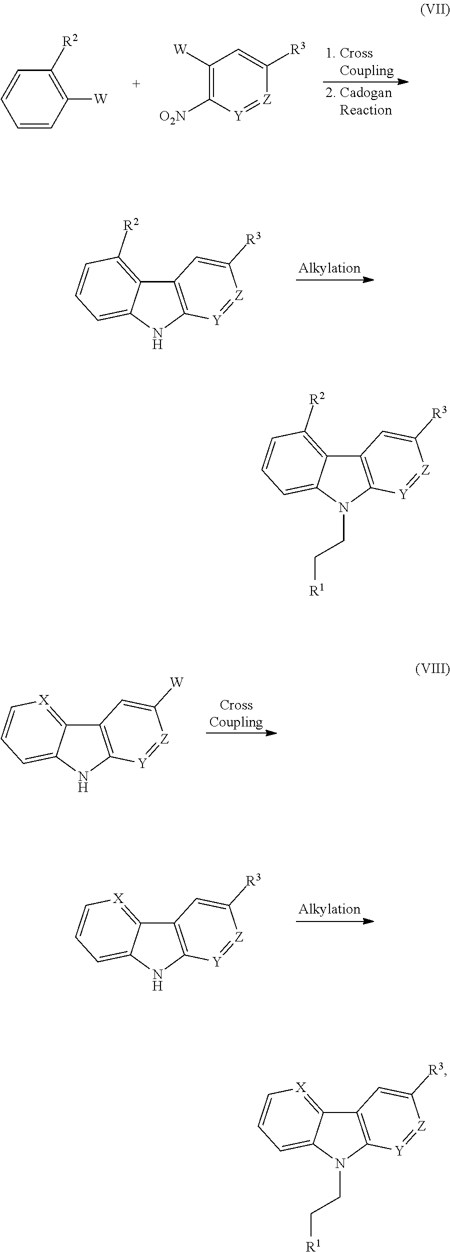

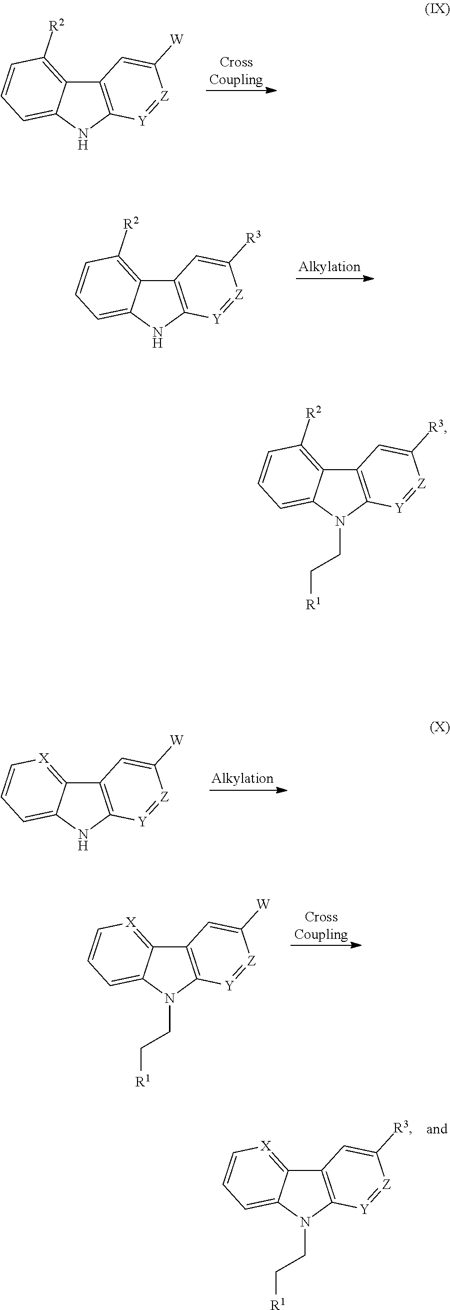

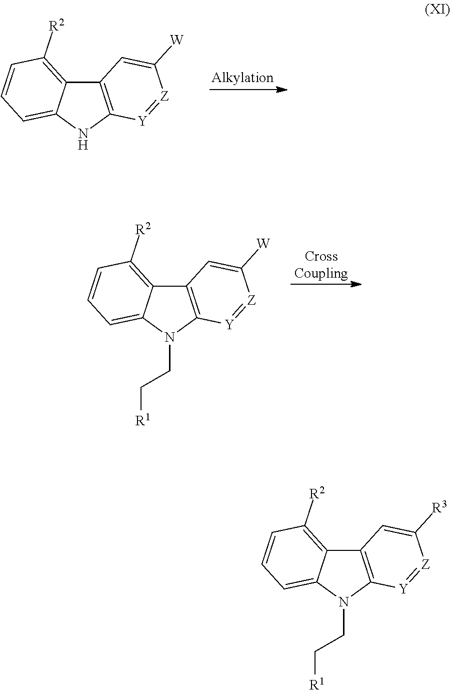

where each W, independently, is a halogen, a trifluoromethanesulfonate, a trialkyltin, a boronic acid, or boronic ester as long as one coupling partner W is a halogen and the other coupling partner W is not a halogen. X, Y, Z, R¹, R², and R³ are as defined herein. The determination of suitable reaction conditions for cross coupling, the Cadogan reaction, alkylation, and other functional group transformations (e.g., metal complex, base, reagents, solvent, reaction time, and reaction temperature) are within the purview of one having skill in the art. In certain circumstances, it may be necessary to form the heterocycles of the present disclosure by well-established condensation reactions. To assemble the coupling partners or further functionalize the aromatic components of the present disclosure it may be necessary to use of electrophilic aromatic substitution reactions, nucleophilic aromatic substitution reactions, anion chemistry, and the like. Other oxidation state and functional groups manipulations are within the purview of one having skill in the art.

More specific, non-limiting, examples of methods to synthesize compounds of the present are illustrated in the examples that follow.

In an aspect, the present disclosure provides a composition comprising at least one compound of the disclosure. Compositions comprising at least one compound of the disclosure include, for example, pharmaceutical preparations.

Compositions comprising a compound of the disclosure and a pharmaceutical carrier can be prepared at a patient's bedside, or by a pharmaceutical manufacture. In either case, the compositions or their ingredient can be provided in any suitable container, such as a sealed sterile vial or ampoule, and may be further packaged to include instruction documents for use by a pharmacist, physician or other health care provider. The compositions can be provided as a liquid, or as a lyophilized or powder form that can be reconstituted if necessary when ready for use. In particular, the compositions can be provided in combination with any suitable delivery form or vehicle, examples of which include, for example, liquids, caplets, capsules, tablets, inhalants or aerosol, etc. The delivery devices may comprise components that facilitate release of the pharmaceutical agents over certain time periods and/or intervals, and can include compositions that enhance delivery of the pharmaceuticals, such as nanoparticle, microsphere or liposome formulations, a variety of which are known in the art and are commercially available. Further, each composition described herein can comprise one or more pharmaceutical agents.

The compositions described herein can include one or more standard pharmaceutically acceptable carriers. Some examples of pharmaceutically acceptable carriers can be found in: Remington: The Science and Practice of Pharmacy (2005) 21 st Edition, Philadelphia, Pa. Lippincott Williams & Wilkins.

Various methods known to those skilled in the art can be used to introduce the compositions of the disclosure to an individual. These methods, for example, intravenous, intratumeral, intramuscular, intracranial, intrathecal, intradermal, subcutaneous, vaginal, rectal, and oral routes. The dose of the composition comprising a compound of the disclosure and a pharmaceutical agent will necessarily be dependent upon the needs of the individual to whom the composition of the disclosure is to be administered. These factors include, for example, the weight, age, sex, medical history, and nature and stage of the disease for which a therapeutic or prophylactic effect is desired. The compositions can be used in conjunction with any other conventional treatment modality designed to improve the disorder for which a desired therapeutic or prophylactic effect is intended, non-limiting examples of which include surgical interventions and radiation therapies. The compositions can be administered once, or over a series of administrations at various intervals determined using ordinary skill in the art, and given the benefit of the present disclosure.

The AR positive or negative cancer cells referred to herein are cancer cells that express a detectable amount of AR protein. “Androgen receptor” (and thus its abbreviation “AR”) is a term well known to those skilled in the art and is used herein to refer to AR protein expressed by human cancer cells, including all isoforms and allelic variants of human AR protein.

In an embodiment, AR positive or negative cancer cells, the growth of which can be inhibited in an individual by practicing the method of the disclosure, are cells that express AR that is specifically recognized by any type of anti-human AR antibody. Anti-human AR antibodies are commercially available. In an embodiment, AR positive or negative cancer cells, the growth of which can be inhibited in an individual by practicing the method of the disclosure, are cells that express AR that can be specifically recognized by the anti-human AR antibody available from BD PharMingen, San Diego, Calif., under catalog number #554225. In an embodiment, a detectable amount of AR protein is an amount of AR protein that can be detected by a Western blot.

In an embodiment, AR positive or negative cancer cells are cells that express AR having the amino acid sequence for GenBank accession no. P10275, Sep. 1, 2009 entry, which is incorporated herein by reference. In alternative embodiments, AR positive or negative cancer cells are cells that express AR having an amino acid sequence that is between 70%-99%, inclusive, and including all integers there between, homologous to the amino acid sequence provided for GenBank accession no. P10275, Sep. 1, 2009. The AR positive or negative cells can by cancer cells that express such an AR having any of such sequences, wherein the AR is detectable by Western blot.

In another embodiment, the AR positive or negative cells are breast cancer cells. The breast cancer cells may be any type of breast cancer cells, provided they are AR positive or negative. The breast cancer cells may be any of ER-, PR-, Her2-, or combinations thereof.

The inhibition of growth of the AR positive or negative cancer cells may be partial inhibition or complete inhibition. Eradication of some or all AR positive or negative cancer cells from an individual is considered to be a type of inhibition of growth of the AR positive or negative cancer cells.

In an aspect, the present disclosure provides a method for treating various androgen receptor positive or negative cancer cells using the compounds as described herein.

In an embodiment, the type of cancer cells are selected from the group consisting of prostate cancer, breast cancer, and hepatocellular carcinoma (HCC), thyroid cancer, glioblastoma, or astrocytoma. In an embodiment, certain compounds are particularly useful against certain types of AR positive cancers. In an embodiment, certain compounds are particularly useful against certain types of AR negative cancers. In another embodiment, certain compounds are particularly useful against certain types of both AR positive and AR negative cancers.

In an aspect, the present disclosure provides a method for reducing the number of AR positive or negative cancer cells in a cell culture using the compounds as described herein.

In an aspect, the present disclosure provides a method for inhibiting the growth of AR positive or negative cancer cells in an individual. The method comprises administering to an individual diagnosed with or suspected of having AR positive or negative cancer a composition comprising a compound capable of inhibiting the growth of or killing AR positive or negative cancer cells. General structures of compounds suitable for use in the disclosure are depicted herein.

In an embodiment, the method of the disclosure comprise administering to an individual diagnosed with or suspected of having AR positive or negative cancer a compound as described herein. For example, the AR positive or negative cancer is prostate cancer, breast cancer, or hepatocellular carcinoma (HCC), thyroid cancer, glioblastoma, or astrocytoma.

In an embodiment of the disclosure, an individual can be identified as a candidate for treatment with a composition comprising an effective amount of a compound as described herein. The individual can be identified as such a candidate by obtaining a biological sample of cancerous tissue from the individual and determining whether or not the cancerous tissue expresses AR. Determining the cancerous tissue expresses AR is indicative that the individual is a candidate for the treatment. Determining that the tissue does not express a detectable amount of AR is indicative that the individual is not a candidate for the treatment. Determining whether the cancerous tissue expresses AR can be performed using any suitable technique, such as immunological techniques. In an embodiment, the disclosure includes transforming AR in a biological sample obtained from the individual into an AR-antibody complex, and detecting the AR-antibody complex using an immunodiagnostic device.

The following examples are presented to illustrate the present disclosure. They are not intended to limiting in any manner.

All synthetic chemistry was performed in standard laboratory glassware unless indicated otherwise in the examples. Commercial reagents were used as received. Analytical LC/MS was performed on an Agilent 1200 system with a variable wavelength detector and Agilent 6140 single quadrupole mass spectrometer, alternating positive and negative ion scans. Retention times were determined from the extracted 220 nm UV chromatogram. ¹H NMR was performed on a Bruker DRX-400 at 400 MHz or a Bruker Avance DRX 500 at 500 MHz. For complicated splitting patterns, the apparent splitting is tabulated. Microwave reactions were performed in a Biotage Initiator using the instrument software to control heating time and pressure. Silica gel chromatography was performed manually, or with an Isco CombiFlash for gradient elutions.

Analytical LC/MS method A:

HPLC column: Kinetex, 2.6 m, C18, 50×2.1 mm, maintained at 40° C. HPLC Gradient: 1.0 mL/min, 95:5:0.1 water:acetonitrile:formic acid to 5:95:0.1 water:acetonitrile:formic acid in 2.0 min, maintaining for 0.5 min. Reported retention times are for method A unless indicated otherwise.

Analytical LC/MS method B was performed on a Shimadzu system with an attached API 165 single quadrupole mass spectrometer. Retention times were determined from the 220 nm chromatogram. HPLC column: Phenomenex, C18, 2.5 m, 20×2 mm, maintained at 25° C. HPLC Gradient: 0.5 mL/min, 95:5:0.02 water:acetonitrile:CF₃COOH to 5:95:0.02 water:acetonitrile:CF₃COOH in 2.9 min, maintaining for 0.9 min.

### Example 1

### Compound 1-1. 3-Bromo-9-propyl-9H-carbazole

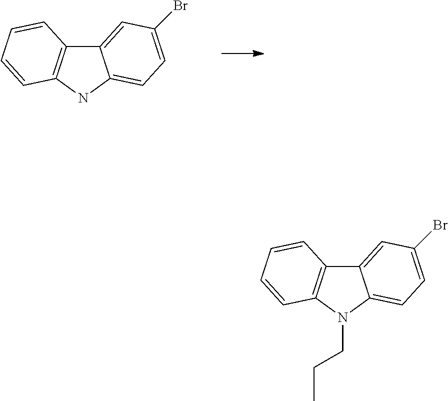 [SMILES: Brc1ccc2[nH]c3ccccc3c2c1 ; CCCn1c2ccccc2c2cc(Br)ccc21]

To a solution of 3-bromo-9H-carbazole (440 mg, 1.79 mmol) and cesium carbonate (1.17 g, 3.57 mmol) in acetonitrile:N,N-dimethylformamide (5:1, 6 mL) was added 1-bromopropane (195 μL, 2.15 mmol) and the mixture stirred at room temperature for 16 h. The mixture was evaporated, diluted with water (10 mL) and extracted with chloroform (3×10 mL). The combined organic layers were dried over sodium sulfate and evaporated to give the title compound (500 mg, 1.73 mmol, 97%) as a colorless oil. LCMS: 97%, Rt 1.75, ESMS m/z 398 (M+H)⁺; (Method B). ¹H NMR (500 MHz, CDCl₃) δ ppm 8.21 (d, J=2.0 Hz, 1H), 8.05 (d, J=7.8 Hz, 1H), 7.54 (dd, J=8.3, 2.0 Hz, 1H), 7.47-7.51 (m, 1H), 7.42 (d, J=8.3 Hz, 1H), 7.29 (d, J=8.3 Hz, 1H), 7.25 (t, J=7.8 Hz, 1H), 4.26 (t, J=7.1 Hz, 2H), 1.92 (sext, J=7.4 Hz, 2H), 0.97 (t, J=7.4 Hz, 3H).

Compounds 1-2-1-8 listed in the table below were prepared in a similar manner from the appropriate carbazole and alkylating agent.

| | | | | | Anal. | |
|---|---|---|---|---|---|---|
| Ex. | Structure | MW | Ion | Rt | Method | Yield |
| 1-2 | 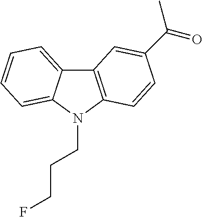 [SMILES: CC(=O)c1ccc2c(c1)c1ccccc1n2CCCF] | 269 | 270 | 1.703 | A | 42 |
| 1-3 | 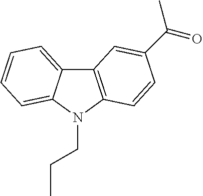 [SMILES: CCCn1c2ccccc2c2cc(C(C)=O)ccc21] | 251 | 252 | 1.850 | A | 47 |
| 1-4 | 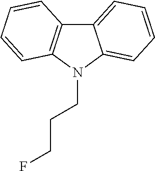 [SMILES: FCCCn1c2ccccc2c2ccccc21] | 227 | 228 | 1.930 | A | 91 |
| 1-5 | 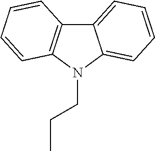 [SMILES: CCCn1c2ccccc2c2ccccc21] | 209 | 210 | 1.770 | A | ~100 |
| 1-6 | 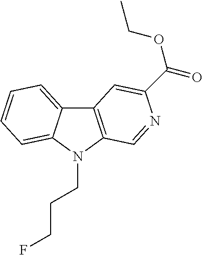 [SMILES: CCOC(=O)c1cc2c3ccccc3n(CCCF)c2cn1] | 300 | 301 | 1.484 | A | 100 |
| 1-7 | 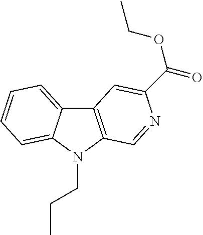 [SMILES: CCCn1c2ccccc2c2cc(C(=O)OCC)ncc21] | 282 | 283 | 1.548 | A | 54 |
| 1-8 | 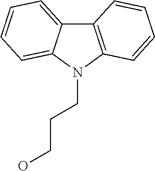 [SMILES: OCCCn1c2ccccc2c2ccccc21] | 225 | 226 | 1.595 | A | 69 |

### Example 2

### Compound 2-1. 9-Propyl-3-thiophen-2-yl-9H-carbazole

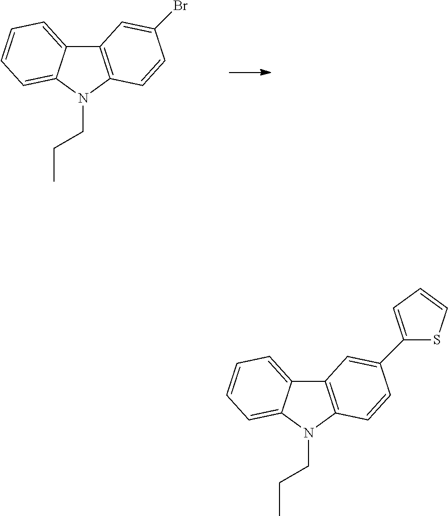 [SMILES: CCCn1c2ccccc2c2cc(Br)ccc21 ; CCCn1c2ccccc2c2cc(-c3cccs3)ccc21]

A biphasic mixture of 3-bromo-9-propyl-9H-carbazole (Compound 1-1, 100 mg, 0.35 mmol), thiophene-2-boronic acid (66 mg, 0.53 mmol), dichloro[1,1′-bis(diphenylphosphino)-ferrocene]palladium(II) (26 mg, 0.035 mmol) and aqueous potassium carbonate (2 M, 350 μL, 0.70 mmol) in 1,4-dioxane (4 mL) was stirred at 100° C. for 16 h. The mixture was evaporated and the residue was purified by column chromatography eluting with hexane. The solid was triturated with cold hexane (1 mL) to give the title compound (46 mg, 0.16 mmol, 46%) as a white powder. LCMS: 100%, Rt 2.190 (Method B), ESMS m/z 292 (M+H)⁺; ¹H NMR (500 MHz, CDCl₃) δ ppm 8.33 (d, J=1.5 Hz, 1H), 8.15 (d, J=7.3 Hz, 1H), 7.74 (dd, J=8.3, 1.5 Hz, 1H), 7.49 (t, J=7.6 Hz, 1H), 7.38-7.45 (m, 2H), 7.35 (d, J=3.9 Hz, 1H), 7.23-7.26 (m, 2H), 7.12 (dd, J=5.1, 3.7 Hz, 1H), 4.30 (t, J=7.1 Hz, 2H), 1.95 (sext, J=7.4 Hz, 2H), 1.00 (t, J=7.4 Hz, 3H).

Compounds 2-2-2-14 listed in the table below were prepared in a similar manner.

| | | | | | Anal. | |
|---|---|---|---|---|---|---|
| | | | | | Meth- | |
| Ex. | Structure | MW | Ion | Rt | od | Yield |
| 2-2 | 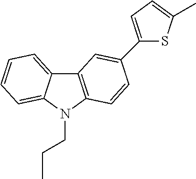 [SMILES: CCCn1c2ccccc2c2cc(-c3ccc(C)s3)ccc21] | 305 | 306 | 2.050 | B | 8 |
| 2-3 | 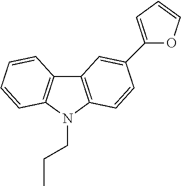 [SMILES: CCCn1c2ccccc2c2cc(-c3ccco3)ccc21] | 275 | 276 | 2.194 | A | 30 |
| 2-4 | 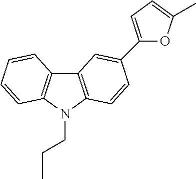 [SMILES: CCCn1c2ccccc2c2cc(-c3ccc(C)o3)ccc21] | 289 | 290 | 2.184 | A | 54 |
| 2-5 | 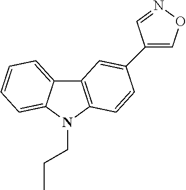 [SMILES: CCCn1c2ccccc2c2cc(-c3cnoc3)ccc21] | 276 | 277 | 1.894 | A | 17 |
| 2-6 | 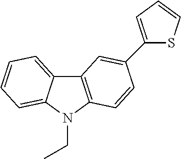 [SMILES: CCn1c2ccccc2c2cc(-c3cccs3)ccc21] | 277 | 278 | 2.194 | A | 42 |
| 2-7 | 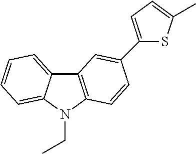 [SMILES: CCn1c2ccccc2c2cc(-c3ccc(C)s3)ccc21] | 291 | 292 | 2.265 | A | 12 |
| 2-8 | 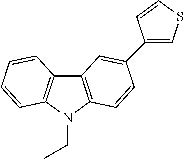 [SMILES: CCn1c2ccccc2c2cc(-c3ccsc3)ccc21] | 277 | 278 | 2.132 | A | 41 |
| 2-9 | 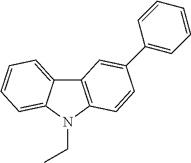 [SMILES: CCn1c2ccccc2c2cc(-c3ccccc3)ccc21] | 271 | 272 | 2.218 | A | 44 |
| 2-10 | 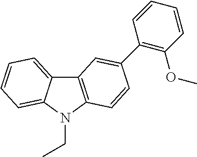 [SMILES: CCn1c2ccccc2c2cc(-c3ccccc3OC)ccc21] | 301 | 302 | 2.159 | A | 42 |
| 2-11 | 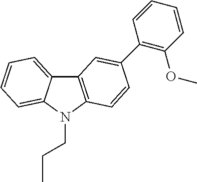 [SMILES: CCCn1c2ccccc2c2cc(-c3ccccc3OC)ccc21] | 315 | 316 | 2.143 | A | 38 |
| 2-12 | 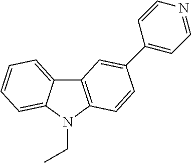 [SMILES: CCn1c2ccccc2c2cc(-c3ccncc3)ccc21] | 272 | 273 | 1.415 | A | 10 |
| 2-13 | 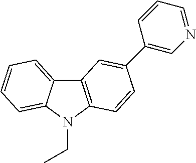 [SMILES: CCn1c2ccccc2c2cc(-c3cccnc3)ccc21] | 272 | 273 | 1.453 | A | 25 |
| 2-14 | 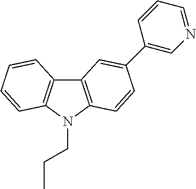 [SMILES: CCCn1c2ccccc2c2cc(-c3cccnc3)ccc21] | 286 | 287 | 1.430 | B | 33 |

### Example 3

### Compound 3-1. 9-Ethyl-3-thiazol-5-yl-9H-carbazole

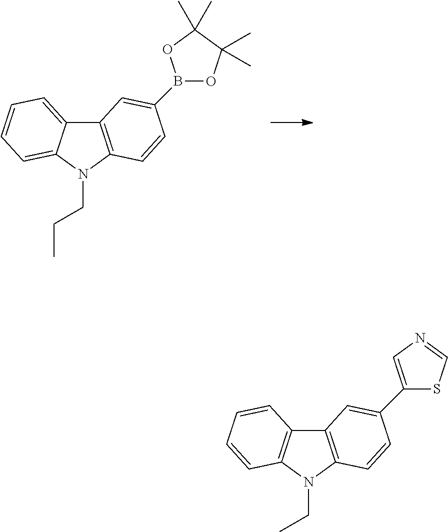 [SMILES: CCCn1c2ccccc2c2cc(B3OC(C)(C)C(C)(C)O3)ccc21 ; CCn1c2ccccc2c2cc(-c3cncs3)ccc21]

A biphasic mixture of 9-ethyl-3-(4,4,5,5-tetramethyl-[1,3,2]dioxaborolan-2-yl)-9H-carbazole (100 mg, 0.31 mmol), 5-bromothiazole (102 mg, 56 μL, 0.62 mmol), tetrakis(triphenylphosphine)palladium(0) (29 mg, 0.025 mmol), aqueous potassium carbonate (2 M, 310 μL, 0.62 mmol) in ethanol (0.8 mL) and toluene (0.4 mL) was stirred at 90° C. for 16 h. The mixture was evaporated and the residue was purified by column chromatography eluting with hexane:ethyl acetate (9:1). The solid was triturated with hexane (1 mL) to give the title compound (46 mg, 0.17 mmol, 53%) as a white powder. LCMS: 97%, Rt 1.884, ESMS m/z 279 (M+H)⁺; ¹H NMR (400 MHz, CDCl₃) δ ppm 8.74 (s, 1H), 8.28 (d, J=1.5 Hz, 1H), 8.14 (d, J=7.8 Hz, 1H), 8.11 (s, 1H), 7.69 (dd, J=8.4, 1.6 Hz, 1H), 7.50 (t, J=7.5 Hz, 1H), 7.39-7.45 (m, 2H), 7.27 (t, J=7.2 Hz, 1H), 4.39 (q, J=7.2 Hz, 2H), 1.46 (t, J=7.3 Hz, 3H).

Compound 3-2 listed in the table below was prepared in a similar manner.

| | | | | | Anal. | |
|---|---|---|---|---|---|---|
| Ex. | Structure | MW | Ion | Rt | Method | Yield |
| 3-2 | 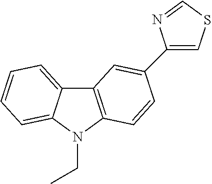 [SMILES: CCn1c2ccccc2c2cc(-c3cscn3)ccc21] | 278 | 279 | 1.890 | A | 32 |

### Example 4

### Compound 4-1. 3-Oxazol-2-yl-9-propyl-9H-carbazole

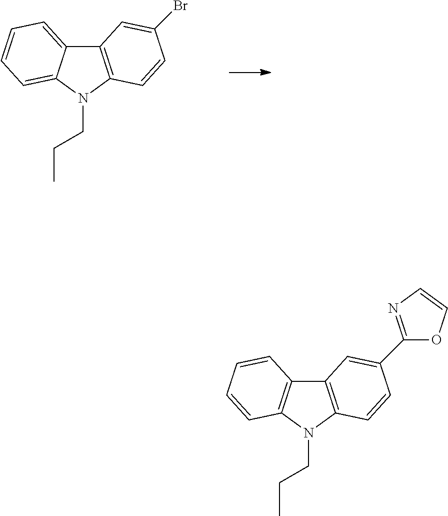 [SMILES: CCCn1c2ccccc2c2cc(Br)ccc21 ; CCCn1c2ccccc2c2cc(-c3ncco3)ccc21]

A mixture of 3-bromo-9-propyl-9H-carbazole (Compound 1-1, 158 mg, 0.55 mmol), 2-(tributylstannyl)-oxazole (294.3 mg, 172 μL, 0.82 mmol), tetrakis(triphenylphosphine) palladium(0) (31 mg, 0.027 mmol) and lithium chloride (70.0 mg, 1.64 mmol) in toluene (5 mL) was heated at reflux for 16 h. The mixture was concentrated and the residue purified by column chromatography eluting with hexane:ethyl acetate (20:1). The solid was triturated with hexane (0.5 mL) to give the title compound (13 mg, 0.047 mmol, 9%) as a white powder. LCMS: 98%, Rt 1.963, ESMS m/z 277 (M+H)⁺; ¹H NMR (400 MHz, DMSO-d₆) δ ppm 8.78 (d, J=1.0 Hz, 1H), 8.30 (d, J=7.8 Hz, 1H), 8.19 (s, 1H), 8.09 (dd, J=8.7, 1.6 Hz, 1H), 7.76 (d, J=8.5 Hz, 1H), 7.67 (d, J=8.3 Hz, 1H), 7.51 (t, J=7.7 Hz, 1H), 7.36 (s, 1H), 7.26 (t, J=7.4 Hz, 1H), 4.41 (t, J=7.0 Hz, 2H), 1.82 (sext, J=7.3 Hz, 2H), 0.89 (t, J=7.4 Hz, 3H).

Compounds 4-2-4-5 listed in the table below were prepared in a similar manner from the corresponding stannanes.

| | | | | | Anal. | |
|---|---|---|---|---|---|---|
| Ex. | Structure | MW | Ion | Rt | Method | Yield |
| 4-2 | 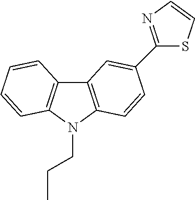 [SMILES: CCCn1c2ccccc2c2cc(-c3nccs3)ccc21] | 292 | 293 | 2.069 | A | 36 |
| 4-3 | 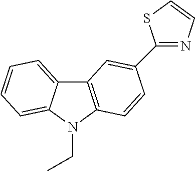 [SMILES: CCn1c2ccccc2c2cc(-c3nccs3)ccc21] | 278 | 279 | 1.940 | A | 26 |
| 4-4 | 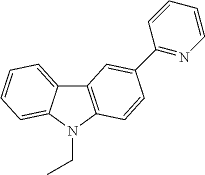 [SMILES: CCn1c2ccccc2c2cc(-c3ccccn3)ccc21] | 272 | 273 | 1.418 | A | 16 |
| 4-5 | 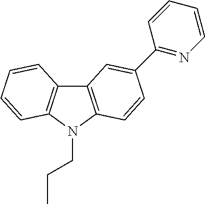 [SMILES: CCCn1c2ccccc2c2cc(-c3ccccn3)ccc21] | 286 | 287 | 1.520 | B | 25 |

### Example 5

### Compound 5-1. 1-(5-Fluoro-9-propyl-9H-carbazol-3-yl)-ethanone

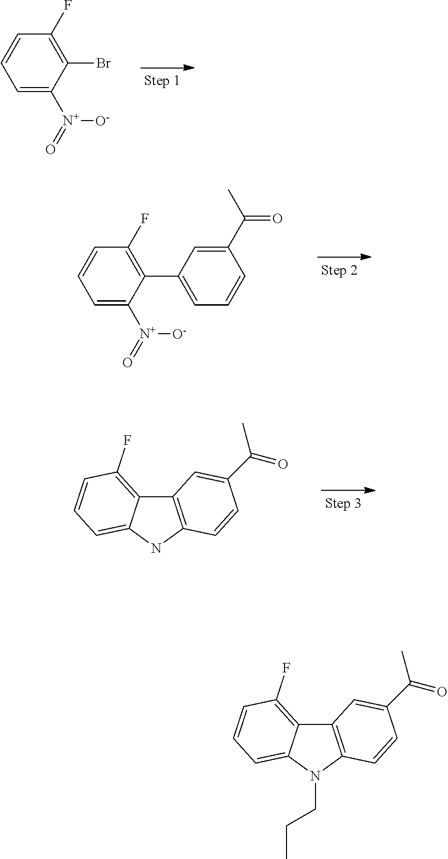 [SMILES: O=[N+]([O-])c1cccc(F)c1Br ; CC(=O)c1cccc(-c2c(F)cccc2[N+](=O)[O-])c1 ; CC(=O)c1ccc2[nH]c3cccc(F)c3c2c1 ; CCCn1c2ccc(C(C)=O)cc2c2c(F)cccc21]

Step 1. Compound 5a-1. 1-(6′-Fluoro-2′-nitrobiphenyl-3-yl)-ethanone. A biphasic mixture of 2-bromo-1-fluoro-3-nitrobenzene (500 mg, 2.27 mmol), 3-acetylphenyl-boronic acid (447 mg, 2.727 mmol), dichloro(1,1′-bis(diphenylphosphino)ferrocene) palladium(II) (166 mg, 0.227 mmol) and aqueous potassium carbonate (2 M, 2.3 mL, 4.54 mmol) in 1,4-dioxane (12 mL) was heated at 120° C. for 1 h under microwave irradiation. The mixture was evaporated and the residue was diluted with water (20 mL). The aqueous phase was extracted with dichloromethane (3×25 mL) and the combined organic layers were dried over sodium sulfate and evaporated. The residue was purified by column chromatography eluting with hexane:ethyl acetate (10:1) to give the title compound (492 mg, 1.90 mmol, 84%) as a yellow powder. LCMS: 99%, Rt 1.591, ESMS m/z 260 (M+H)⁺.

Compounds 5a-2-5a-14 listed in the table below were prepared in a similar manner from the appropriate aryl bromide and boronic acid.

| | | | | | Anal. | |
|---|---|---|---|---|---|---|
| Ex. | Structure | MW | Ion | Rt | Method | Yield |
| 5a-2 | 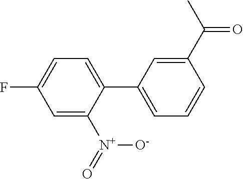 [SMILES: CC(=O)c1cccc(-c2ccc(F)cc2[N+](=O)[O-])c1] | 227 | 228 | 1.499 | A | 38 |
| 5a-3 | 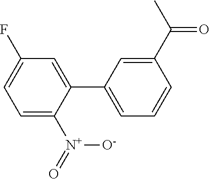 [SMILES: CC(=O)c1cccc(-c2cc(F)ccc2[N+](=O)[O-])c1] | 259 | 260 | 1.571 | A | 34 |
| 5a-4 | 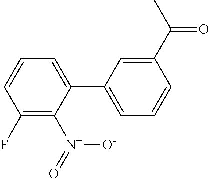 [SMILES: CC(=O)c1cccc(-c2cccc(F)c2[N+](=O)[O-])c1] | 259 | 260 | 1.622 | A | 95 |
| 5a-5 | 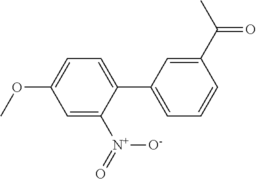 [SMILES: COc1ccc(-c2cccc(C(C)=O)c2)c([N+](=O)[O-])c1] | 271 | 272 | 1.660 | A | 94 |
| 5a-6 | 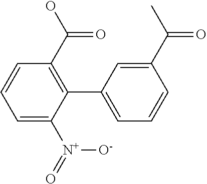 [SMILES: CC(=O)c1cccc(-c2c(C(=O)O)cccc2[N+](=O)[O-])c1] | 285 | 286 | 1.321 | A | 95 |
| 5a-7 | 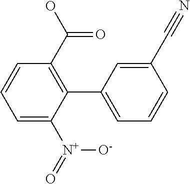 [SMILES: N#Cc1cccc(-c2c(C(=O)O)cccc2[N+](=O)[O-])c1] | 268 | 269 | 1.320 | A | crude |
| 5a-8 | 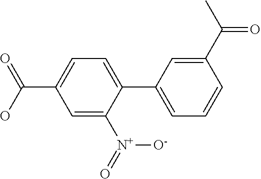 [SMILES: CC(=O)c1cccc(-c2ccc(C(=O)O)cc2[N+](=O)[O-])c1] | 285 | 286 | 1.375 | A | 93 |
| 5a-9 | 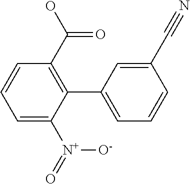 [SMILES: N#Cc1cccc(-c2c(C(=O)O)cccc2[N+](=O)[O-])c1] | 268 | 269 | 1.320 | A | crude |
| 5a-10 | 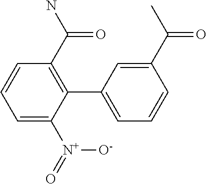 [SMILES: CC(=O)c1cccc(-c2c(C(N)=O)cccc2[N+](=O)[O-])c1] | 284 | 285 | 1.125 | A | 79 |
| 5a-11 | 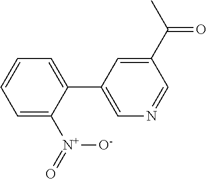 [SMILES: CC(=O)c1cncc(-c2ccccc2[N+](=O)[O-])c1] | 242 | 243 | 1.263 | A | 88 |
| 5a-12 | 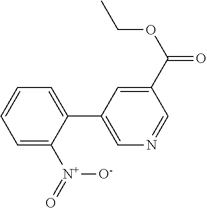 [SMILES: CCOC(=O)c1cncc(-c2ccccc2[N+](=O)[O-])c1] | 272 | 273 | 1.545 | A | 72 |
| 5a-13 | 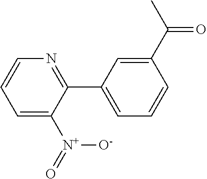 [SMILES: CC(=O)c1cccc(-c2ncccc2[N+](=O)[O-])c1] | 242 | 243 | 1.297 | A | 97 |
| 5a-14 | 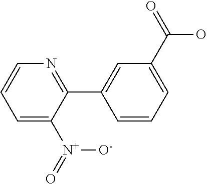 [SMILES: O=C(O)c1cccc(-c2ncccc2[N+](=O)[O-])c1] | 244 | 245 | 1.192 | A | 58 |

Step 2. Compound 5b-1. 1-(5-Fluoro-9H-carbazol-3-yl)-ethanone. A mixture of 1-(6′-fluoro-2′-nitrobiphenyl-3-yl)ethanone (Compound 5a-1, 490 mg, 1.89 mmol) and triphenylphosphine (1.49 g, 5.67 mmol) in chlorobenzene (12 mL) was heated under microwave irradiation at 200° C. for 2 h. The mixture was evaporated and the residue purified by column chromatography eluting with hexane:ethyl acetate (9:1 to 3:1) to give the title compound (200 mg, 0.88 mmol, 46%) as a light brown powder. LCMS: 85%, Rt 1.508, ESMS m/z 228 (M+H)⁺.

Compounds 5b-2-5b-12 listed in the table below were prepared in a similar manner from the appropriate nitroaryl compound.

| | | | | | Anal. | |
|---|---|---|---|---|---|---|
| Ex. | Structure | MW | Ion | Rt | Method | Yield |
| 5b-2 | 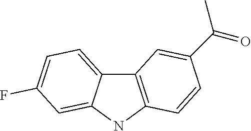 [SMILES: CC(=O)c1ccc2[nH]c3cc(F)ccc3c2c1] | 227 | 228 | 1.499 | A | 38 |
| 5b-3 | 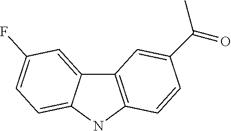 [SMILES: CC(=O)c1ccc2[nH]c3ccc(F)cc3c2c1] | 227 | 228 | 1.702 | A | 42 |
| 5b-4 | 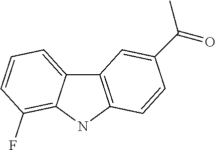 [SMILES: CC(=O)c1ccc2[nH]c3c(F)cccc3c2c1] | 227 | 228 | 1.549 | A | 16 |
| 5b-5 | 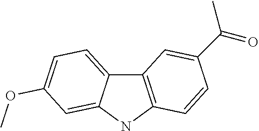 [SMILES: COc1ccc2c(c1)[nH]c1ccc(C(C)=O)cc12] | 239 | 240 | 1.44 | A | 38 |
| 5b-6 | 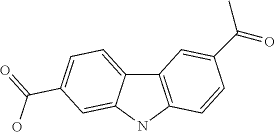 [SMILES: CC(=O)c1ccc2[nH]c3cc(C(=O)O)ccc3c2c1] | 253 | 254 | 1.287 | A | 6 |
| 5b-7 | 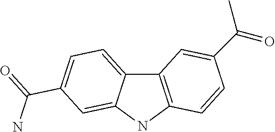 [SMILES: CC(=O)c1ccc2[nH]c3cc(C(N)=O)ccc3c2c1] | 252 | 253 | 1.117 | A | 24 |
| 5b-8 | 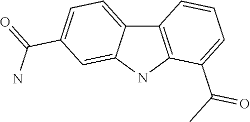 [SMILES: CC(=O)c1cccc2c1[nH]c1cc(C(N)=O)ccc12] | 252 | 253 | 1.242 | A | 46 |
| 5b-9 | 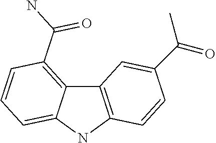 [SMILES: CC(=O)c1ccc2[nH]c3cccc(C(N)=O)c3c2c1] | 252 | 253 | 1.074 | A | 16 |
| 5b-10 | 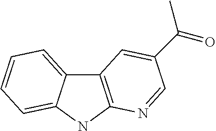 [SMILES: CC(=O)c1cnc2[nH]c3ccccc3c2c1] | 210 | 211 | 1.291 | A | 23 |
| 5b-11 | 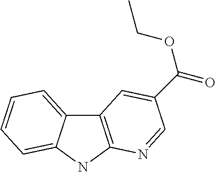 [SMILES: CCOC(=O)c1cnc2[nH]c3ccccc3c2c1] | 240 | 241 | 1.578 | A | 68 |
| 5b-12 | 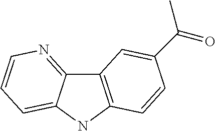 [SMILES: CC(=O)c1ccc2[nH]c3cccnc3c2c1] | 210 | 211 | 0.841 | A | 26 |

Step 3. Compound 5-1. 1-(5-Fluoro-9-propyl-9H-carbazol-3-yl)-ethanone. To a suspension of 1-(5-fluoro-9H-carbazol-3-yl)-ethanon2e (Compound 5b-1, 100 mg, 0.44 mmol) and sodium hydride (21 mg, 0.88 mmol) in N,N-dimethylformamide (500 μL) was added 1-bromopropane (81 mg, 60 μL, 0.66 mmol) and the mixture stirred at room temperature for 1 h. The mixture was evaporated, diluted with water (10 mL) and extracted with dichloromethane (2×10 mL). The combined organic layers were dried over sodium sulfate and evaporated. The residue was purified by column chromatography eluting with hexane:ethyl acetate (20:1). The product was triturated with hexane (1 mL) to give the title compound (22 mg, 0.082 mmol, 18%) as an off-white powder. LCMS: 99%, Rt 1.942, ESMS m/z 270 (M+H)⁺; ¹H NMR (300 MHz, CDCl₃) δ ppm 8.82 (d, J=1.1 Hz, 1H), 8.17 (dd, J=8.7, 1.7 Hz, 1H), 7.36-7.50 (m, 2H), 7.22 (d, J=8.1 Hz, 1H), 6.97 (dd, J=9.6, 8.3 Hz, 1H), 4.30 (t, J=7.2 Hz, 2H), 2.73 (s, 3H), 1.95 (sext, J=7.3 Hz, 2H), 0.98 (t, J=7.4 Hz, 3H).

Compounds 5-2-5-13 listed in the table below were prepared in a similar manner from the appropriate carbazole.

| | | | | | Anal. | |
|---|---|---|---|---|---|---|
| Ex. | Structure | MW | Ion | Rt | Method | Yield |
| 5-2 | 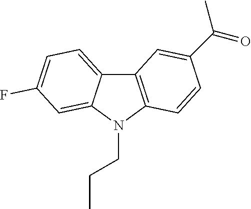 [SMILES: CCCn1c2ccc(C(C)=O)cc2c2ccc(F)cc21] | 269 | 270 | 1.832 | A | 39 |
| 5-3 | 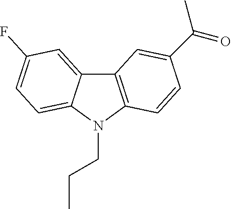 [SMILES: CCCn1c2ccc(F)cc2c2cc(C(C)=O)ccc21] | 269 | 270 | 1.879 | A | 15 |
| 5-4 | 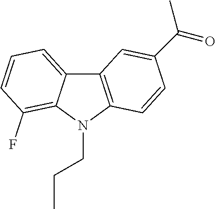 [SMILES: CCCn1c2ccc(C(C)=O)cc2c2cccc(F)c21] | 269 | 270 | 1.970 | A | 34 |
| 5-5 | 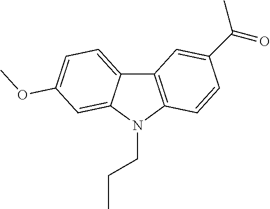 [SMILES: CCCn1c2ccc(C(C)=O)cc2c2ccc(OC)cc21] | 281 | 282 | 1.774 | A | 58 |
| 5-6 | 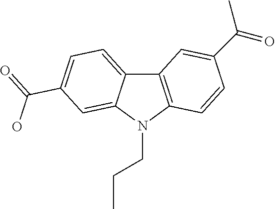 [SMILES: CCCn1c2ccc(C(C)=O)cc2c2ccc(C(=O)O)cc21] | 295 | 296 | 1.567 | A | 38 |
| 5-7 | 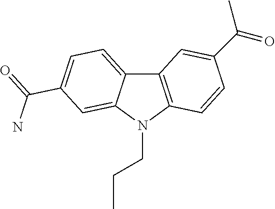 [SMILES: CCCn1c2ccc(C(C)=O)cc2c2ccc(C(N)=O)cc21] | 294 | 295 | 1.385 | A | 44 |
| 5-8 | 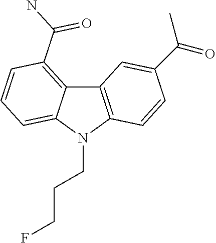 [SMILES: CC(=O)c1ccc2c(c1)c1c(C(N)=O)cccc1n2CCCF] | 312 | 313 | 1.291 | A | |
| 5-9 | 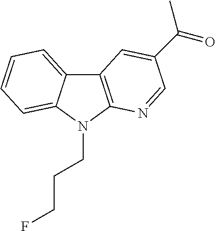 [SMILES: CC(=O)c1cnc2c(c1)c1ccccc1n2CCCF] | 270 | 271 | 1.658 | A | 18 |
| 5-10 | 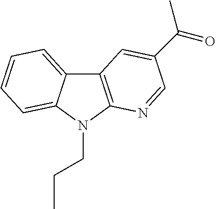 [SMILES: CCCn1c2ccccc2c2cc(C(C)=O)cnc21] | 252 | 253 | 1.682 | A | 27 |
| 5-11 | 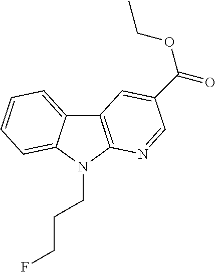 [SMILES: CCOC(=O)c1cnc2c(c1)c1ccccc1n2CCCF] | 300 | 301 | 1.865 | A | 34 |
| 5-12 | 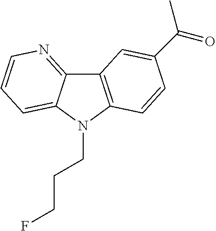 [SMILES: CC(=O)c1ccc2c(c1)c1ncccc1n2CCCF] | 270 | 271 | 1.161 | A | 82 |
| 5-13 | 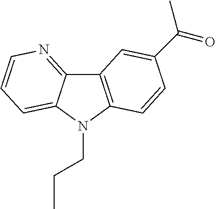 [SMILES: CCCn1c2ccc(C(C)=O)cc2c2ncccc21] | 252 | 253 | 1.245 | A | 38 |

### Example 6

### Compound 6-1. 1-(5-Methoxy-9-propyl-9H-carbazol-3-yl)-ethanone

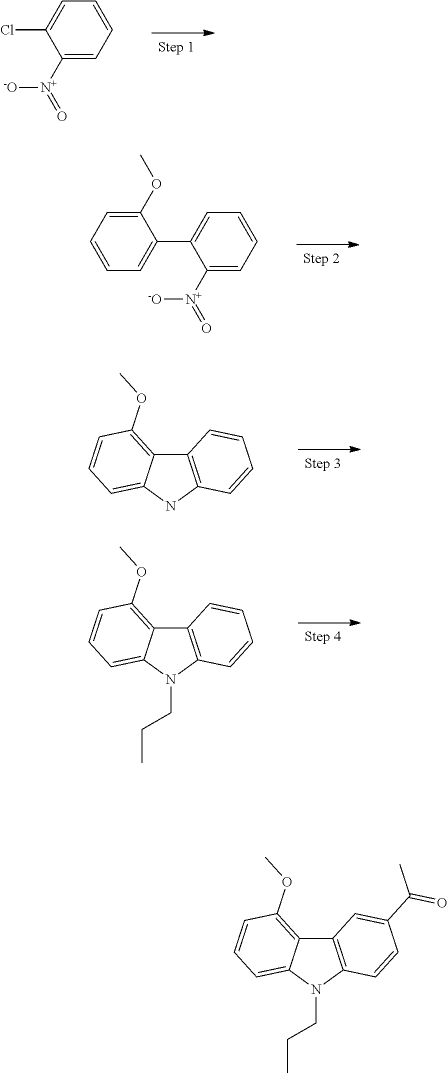

Step 1. Compound 6a-1. 2′-Methoxy-2-nitrobiphenyl. A biphasic mixture of 1-chloro-2-nitrobenzene (500 mg, 3.17 mmol), 2-methoxyphenylboronic acid (579 mg, 3.81 mmol), dichloro[1,1′-bis(diphenylphosphino)ferrocene]palladium(II) (116 mg, 0.159 mmol) and aqueous potassium carbonate (2 M, 3.17 mL, 6.34 mmol) in 1,4-dioxane (12 mL) was stirred at 100° C. for 3 h. The mixture was evaporated and the residue diluted with water (20 mL). The aqueous layer was extracted with dichloromethane (3×25 mL). The combined organic layers were dried over sodium sulfate and evaporated. The residue was purified by column chromatography eluting with hexane:ethyl acetate (100:1) to give the title compound (590 mg, 2.57 mmol, 81%) as a light brown powder. LCMS: 90%, Rt 1.707, ESMS m/z 230 (M+H)+.

Compounds 6a-2-6a-3 listed in the table below were prepared in a similar manner.

| | | | | | Anal. | |
|---|---|---|---|---|---|---|
| Ex. | Structure | MW | Ion | Rt | Method | Yield |
| 6a-2 | 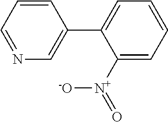 | 200 | 200 | | A | 54 |
| 6a-3 | 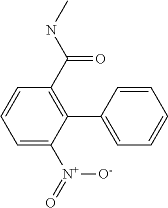 [SMILES: CNC(=O)c1cccc([N+](=O)[O-])c1-c1ccccc1] | 256 | 257 | 1.236 | A | 87 |

Step 2. Compound 6b-1. 4-Methoxy-9H-carbazole. A mixture of 2′-methoxy-2-nitrobiphenyl (Compound 6a-1, 520 mg, 2.26 mmol) and triphenylphosphine (1.48 g, 5.67 mmol) in chlorobenzene (6 mL) was heated at 200° C. for 2 h under microwave irradiation. The mixture was evaporated and the residue purified by column chromatography eluting with hexane:ethyl acetate (20:1) to give the title compound (335 mg, 1.70 mmol, 75%) as a white powder. LCMS: 88%, RT 1.678, ESMS m/z 198 (M+H)+.

Compounds 6b-2-6b-4 listed in the table below were prepared in a similar manner.

| | | | | | Anal. | |
|---|---|---|---|---|---|---|
| Ex. | Structure | MW | Ion | Rt | Method | Yield |
| 6b-2 | 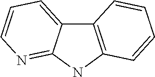 [SMILES: c1ccc2c(c1)[nH]c1ncccc12] | 168 | 169 | 1.066 | A | 54 |
| 6b-3 | 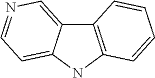 [SMILES: c1ccc2c(c1)[nH]c1ccncc12] | 168 | 169 | 0.717 | A | 31 |
| 6b-4 | 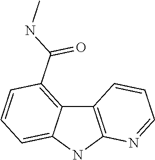 [SMILES: CNC(=O)c1cccc2[nH]c3ncccc3c12] | 224 | 225 | 1.184 | A | 78 |

Step 3. Compound 6c-1. 4-Methoxy-9-propyl-9H-carbazole. To a suspension of 4-methoxy-9H-carbazole (Compound 6b-1, 335 mg, 1.70 mmol) and cesium carbonate (1.10 g, 3.40 mmol) in N,N-dimethylformamide (8 mL) was added 1-bromopropane (308 μL, 3.40 mmol) and the mixture stirred at 50° C. for 16 h. The mixture was evaporated and the residue was diluted with water (10 mL) and extracted with dichloromethane (2×10 mL). The combined organic layers were dried over sodium sulfate and evaporated. The residue was purified by column chromatography eluting with hexane:ethyl acetate (20:1) to give the title compound (390 mg, 1.62 mmol, 95%) as yellow oil. LCMS: 82%, Rt 2.078, ESMS m/z 240 (M+H)⁺.

Compounds 6c-2-6c-3 listed in the table below were prepared in a similar manner.

| | | | | | Anal. | |
|---|---|---|---|---|---|---|
| Ex. | Structure | MW | Ion | Rt | Method | Yield |
| 6c-2 | 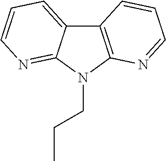 [SMILES: CCCn1c2ncccc2c2cccnc21] | 210 | 211 | 1.672 | A | 76 |
| 6c-3 | 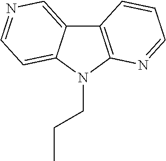 [SMILES: CCCn1c2ccncc2c2cccnc21] | 210 | 211 | 1.122 | A | 32 |

Step 4. Compound 6-1. 1-(5-Methoxy-9-propyl-9H-carbazol-3-yl)-ethanone. To a solution of 4-methoxy-9-propyl-9H-carbazole (Compound 6c-1, 390 mg, 1.62 mmol) in dichloromethane (4 mL) was added aluminum chloride (434 mg, 3.26 mmol). To this mixture was added a solution of acetyl chloride (139 μL, 1.96 mmol) in dichloromethane (4 mL) dropwise and the mixture stirred at room temperature for 4 h. The reaction mixture was diluted with dichloromethane (10 mL) and washed with saturated sodium bicarbonate (10 mL). The aqueous layer was extracted with dichloromethane (2×5 mL) and the combined organic layers were dried over sodium sulfate and evaporated. The residue was purified by column chromatography eluting with hexane:ethyl acetate (10:1). The product was triturated with hexane:diethyl ether (1:1, 2 mL) to give the title compound (71 mg, 0.25 mmol, 15%) as a white powder. LCMS: 100%, Rt 1.907, ESMS m/z 282 (M+H). ¹H NMR (500 MHz, CDCl₃) δ ppm 8.96 (d, J=1.5 Hz, 1H), 8.12 (dd, J=8.8, 2.0 Hz, 1H), 7.44 (t, J=8.3 Hz, 1H), 7.40 (d, J=8.3 Hz, 1H), 7.07 (d, J=8.3 Hz, 1H), 6.76 (d, 1H), 4.29 (t, J=7.1 Hz, 2H), 4.13 (s, 3H), 2.74 (s, 3H), 1.94 (sext, J=7.3 Hz, 2H), 0.98 (t, J=7.3 Hz, 3H)

Compounds 6-2-6-3 listed in the table below were prepared in a similar manner. Compound 6-4 was prepared by a similar procedure where Step 3 and Step 4 were performed in the reverse order.

| | | | | | Anal. | |
|---|---|---|---|---|---|---|
| Ex. | Structure | MW | Ion | Rt | Method | Yield |
| 6-2 | 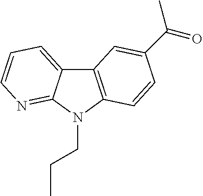 [SMILES: CCCn1c2ccc(C(C)=O)cc2c2cccnc21] | 252 | 253 | 1.590 | A | 63 |
| 6-3 | 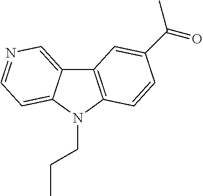 [SMILES: CCCn1c2ccncc2c2cc(C(C)=O)ccc21] | 252 | 253 | 1.043 | A | 32 |
| 6-4 | 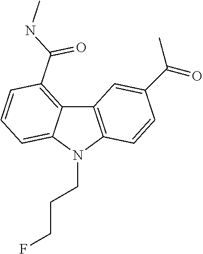 [SMILES: CNC(=O)c1cccc2c1c1cc(C(C)=O)ccc1n2CCCF] | 326 | 327 | 1.344 | A | |

### Example 7

### Compound 7-1. 9-(3-Fluoropropyl)-9H-pyrido[2,3-b]indole-3-carboxamide

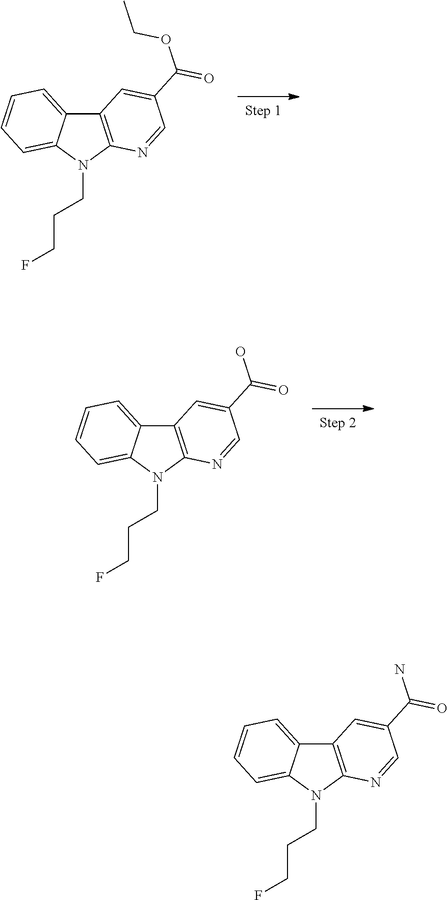 [SMILES: CCOC(=O)c1cnc2c(c1)c1ccccc1n2CCCF ; O=C(O)c1cnc2c(c1)c1ccccc1n2CCCF ; NC(=O)c1cnc2c(c1)c1ccccc1n2CCCF]

Step 1. Compound 7a-1. 9-(3-Fluoropropyl)-9H-pyrido[2,3-b]indole-3-carboxylic acid. To a solution of 9-(3-fluoropropyl)-9H-pyrido[2,3-b]indole-3-carboxylic acid ethyl ester (Compound 5-11, 557 mg, 1.86 mmol) in 1,4-dioxane (12 mL) was added 20% aqueous sodium hydroxide solution (6 mL) and the mixture stirred at 50° C. for 18 h. The mixture was evaporated and the residue was partitioned between water (10 mL) and chloroform (10 mL), and the precipitate was collected to give the title compound (355 mg, 1.31 mmol, 70%) as an off-white powder. LCMS: 100%, Rt 1.467, ESMS m/z 273 (M+H)⁺.

Step 2. Compound 7-1. 9-(3-Fluoropropyl)-9H-pyrido[2,3-b]indole-3-carboxamide. To a solution of 9-(3-fluoropropyl)-9H-pyrido[2,3-b]indole-3-carboxylic acid (Compound 7a-1, 140 mg, 0.514 mmol) in N,N-dimethylformamide (2 mL) was added 2-(7-aza-1H-benzotriazole-1-yl)-1,1,3,3-tetramethyluronium hexafluorophosphate (HATU, 196 mg, 0.514 mmol), triethylamine (143 μL, 1.028 mmol) and a solution of ammonia in 1,4-dioxane (3 M, 2 mL) and the mixture stirred at room temperature for 1 h. The mixture was evaporated, the residue was diluted with ethyl acetate (5 mL) and the precipitate was collected. The crude product was purified by column chromatography eluting with chloroform to give the title compound (26 mg, 0.10 mmol, 18%) as a white powder. LCMS: 99%, Rt 1.309, ESMS m/z 272 (M+H)⁺; ¹H NMR (300 MHz, DMSO-d₆) δ ppm 9.02 (br. s, 2H), 8.25 (d, J=7.5 Hz, 1H), 8.10 (br. s, 1H), 7.73 (d, J=8.3 Hz, 1H), 7.59 (br. s, 1H), 7.39-7.49 (m, 1H), 7.24-7.39 (m, 1H), 4.62 (br. s, 2H), 4.49 (dt, J=47.2, 6.0 Hz, 2H), 2.21 (dquint, J=26.4, 6.0 Hz, 2H)

### Example 8

### Compound 8-1. 3-[1,3,4]Oxadiazol-2-yl-9-propyl-9H-carbazole

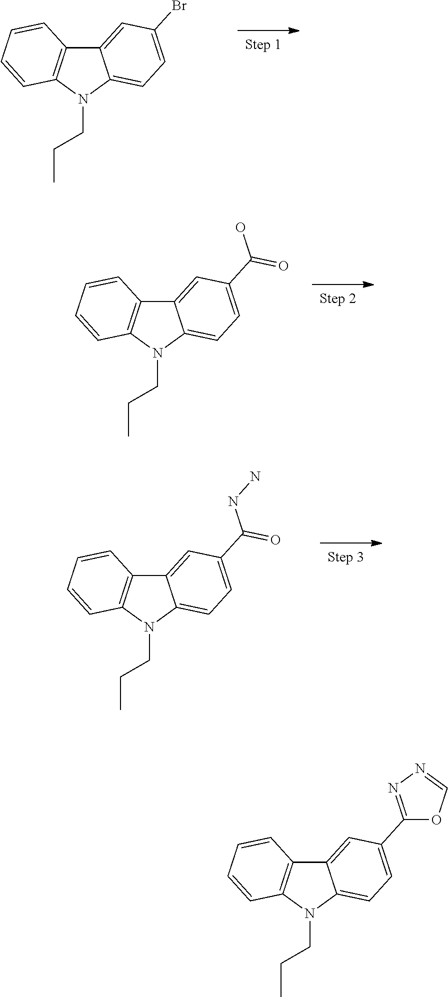 [SMILES: CCCn1c2ccccc2c2cc(Br)ccc21 ; CCCn1c2ccccc2c2cc(C(=O)O)ccc21 ; CCCn1c2ccccc2c2cc(C(=O)NN)ccc21 ; CCCn1c2ccccc2c2cc(-c3nnco3)ccc21]

Step 1. Compound 8a-1. 9-Propyl-9H-carbazole-3-carboxylic acid. To a solution of n-butyllithium (1.6 M in hexanes, 9.13 mL, 14.6 mmol) at −78° C. under argon was added a solution of 9-propyl-3-bromocarbazole (Compound 1-1, 3.5 g, 12.1 mmol) in dry tetrahydrofuran (80 mL) and the mixture stirred for 30 min. Carbon dioxide was bubbled through the solution for 15 min. The reaction mixture was allowed to warm to room temperature and evaporated. The residue was partitioned between ethyl acetate (50 mL) and water (25 mL) and the layers were separated. The aqueous layer was extracted with ethyl acetate (2×40 mL). The combined organic layers were dried over sodium sulfate and evaporated to give the title compound (2.16 g, 8.54 mmol, 70%) as an off-white powder. LCMS: 100%, Rt 1.630, ESMS m/z 254 (M+H)⁺.

Step 2. Compound 8b-1. 9-Propyl-9H-carbazole-3-carbohydrazide. A mixture of 9-propyl-9H-carbazole-3-carboxylic acid (Compound 8a-1, 500 mg, 1.97 mmol), N,N,N′,N′-tetramethyl-O-(7-azabenzotriazol-1-yl)uronium hexafluorophosphate (HATU; 900 mg, 2.37 mmol) and N-methylmorpholine (435 μL, 3.95 mmol) in acetonitrile (20 mL) was stirred at room temperature for 1 h. Hydrazine hydrate (191 μL, 3.95 mmol) was added and the solution was stirred at room temperature for 16 h. The reaction mixture was evaporated and the residue was taken up in ethyl acetate (20 mL) and 10% aqueous sodium hydroxide (15 mL). The layers were separated, and the organic layer was washed with water (2×10 mL), dried over sodium sulfate and evaporated. The product (521 mg, 1.95 mmol) was used without purification. LCMS: 51%, Rt 1.474, ESMS m/z 268 (M+H)⁺.

Compound 8b-2 listed in the table below was prepared in a similar manner.

| | | | | | Anal. | |
|---|---|---|---|---|---|---|
| Ex. | Structure | MW | Ion | Rt | Method | Yield |
| 8b-2 | 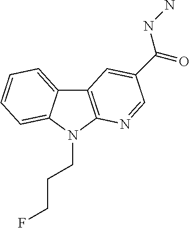 [SMILES: NNC(=O)c1cnc2c(c1)c1ccccc1n2CCCF] | 286 | 287 | 1.187 | A | 71 |

Step 3. Compound 8-1. 3-[1,3,4]Oxadiazol-2-yl-9-propylcarbazole. A solution of 9-propyl-9H-carbazole-3-carboxylic acid hydrazide (Compound 8b-1, 421 mg, 1.57 mmol) in formic acid (3.15 mL) was stirred at 80° C. for 1 h. The reaction mixture was evaporated and the residue was crystallized from acetonitrile to give 9-propyl-9H-carbazole-3-(N-formyl)hydrazide as a white solid. 67% (311 mg, 1.05 mmol), LCMS: 100%, (M−1)⁻=294. A mixture of this intermediate (110 mg, 0.37 mmol) and phosphorus pentoxide (52 mg, 0.37 mmol) in xylene (5.5 mL) was stirred at 140° C. for 1 h. The mixture was evaporated and the residue partitioned between water (10 mL) and dichloromethane (10 mL). The layers were separated and the organic layer was dried over sodium sulfate and evaporated. The crude product was purified by column chromatography eluting with dichloromethane:methanol (100:0 to 98:2) to give the title compound (28 mg, 0.10 mmol, 27%) as a white powder. LCMS: 96%, Rt 1.721, ESMS m/z 278 (M+H)⁺; ¹H NMR (500 MHz, CDCl₃) δ ppm 8.84 (d, J=1.5 Hz, 1H), 8.48 (s, 1H), 8.20 (dd, J=8.6, 1.7 Hz, 1H), 8.18 (d, J=7.3 Hz, 1H), 7.50-7.57 (m, 2H), 7.47 (d, J=7.8 Hz, 1H), 7.32 (t, J=7.3 Hz, 1H), 4.34 (t, J=7.1 Hz, 2H), 1.96 (sext, J=7.4 Hz, 2H), 1.01 (t, J=7.3 Hz, 3H).

Compounds 8-2-8-4 listed in the table below were prepared in a similar manner, using triethyl orthoformate or triethyl orthoacetate in a one-step procedure and phosphorus pentasulfide when appropriate.

| | | | | | Anal. | |
|---|---|---|---|---|---|---|
| Ex. | Structure | MW | Ion | Rt | Method | Yield |
| 8-2 | 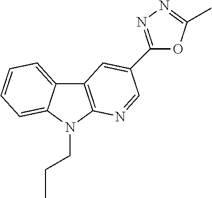 [SMILES: CCCn1c2ccccc2c2cc(-c3nnc(C)o3)cnc21] | 291 | 292 | 1.774 | A | 28 |
| 8-3 | 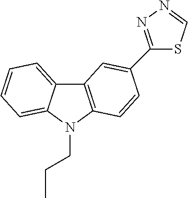 [SMILES: CCCn1c2ccccc2c2cc(-c3nncs3)ccc21] | 293 | 294 | 1.790 | A | 24 |
| 8-4 | 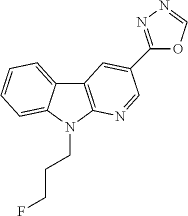 [SMILES: FCCCn1c2ccccc2c2cc(-c3nnco3)cnc21] | 296 | 297 | 1.526 | A | 50 |

### Example 9

### Compound 9-1. 9-(3-Fluoropropyl)-3-[1,3,4]oxadiazol-2-yl-carbazole

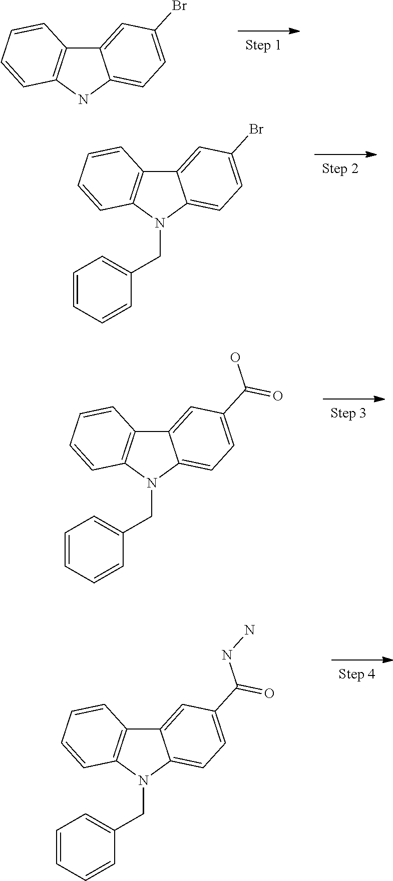 [SMILES: Brc1ccc2[nH]c3ccccc3c2c1 ; Brc1ccc2c(c1)c1ccccc1n2Cc1ccccc1 ; O=C(O)c1ccc2c(c1)c1ccccc1n2Cc1ccccc1 ; NNC(=O)c1ccc2c(c1)c1ccccc1n2Cc1ccccc1]

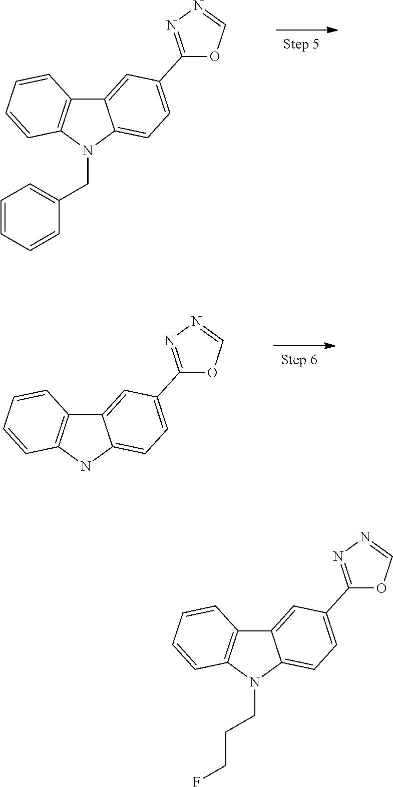 [SMILES: c1ccc(Cn2c3ccccc3c3cc(-c4nnco4)ccc32)cc1 ; c1ccc2c(c1)[nH]c1ccc(-c3nnco3)cc12 ; FCCCn1c2ccccc2c2cc(-c3nnco3)ccc21]

Step 1. Compound 9a-1. 9-Benzyl-3-bromocarbazole. To a solution of 3-bromo-9H-carbazole (1.50 g, 6.10 mmol) in dry N,N-dimethylformamide (15 mL) was added sodium hydride (60% in mineral oil; 488 mg, 12.2 mmol) and the mixture stirred at room temperature for 30 min. Benzyl bromide (1.1 mL, 9.14 mmol) was added and the suspension was stirred at room temperature for 90 min. The reaction mixture was evaporated and the residue taken up in chloroform (20 mL) and water (10 mL). The layers were separated and the aqueous layer was extracted with chloroform (2×20 mL). The combined organic layers were dried over sodium sulfate and evaporated to give the title compound (2.05 g, 6.10 mmol, ca. 100%) as an off-white powder. The crude product was used in the next step without further purification. LCMS: 95%, Rt 2.141, ESMS m/z 336 (M+H)⁺.

Step 2. Compound 9b-1. 9-Benzylcarbazole-3-carboxylic acid. To a solution of n-butyllithium (1.6 M in hexanes, 5.40 mL, 8.64 mmol) at −78° C. under argon was added a solution of 9-benzyl-3-bromocarbazole (Compound 6a-1, 2.05 g, 6.10 mmol) in dry tetrahydrofuran (70 mL) and the mixture stirred at −78° C. for 30 min. Carbon dioxide was bubbled through the solution for 15 min. The reaction mixture was allowed to warm to room temperature then evaporated. The residue was taken up in ethyl acetate (50 mL) and water (25 mL), the layers were separated and the aqueous layer was extracted with ethyl acetate (2×40 mL). The combined organic layers were dried over sodium sulfate and evaporated to give the title compound (1.66 g, 5.51 mmol, 76%) as an off-white powder. LCMS: 100%, Rt 1.702, ESMS m/z 302 (M+H)⁺.

Step 3. Compound 9c-1. 9-Benzylcarbazole-3-carboxylic acid hydrazide. A mixture of 9-benzylcarbazole-3-carboxylic acid (Compound 9b-1, 1.23 g, 4.09 mmol), N,N,N′,N′-tetramethyl-O-(7-azabenzotriazol-1-yl)uronium hexafluorophosphate (HATU; 1.87 g, 4.91 mmol) and N-methylmorpholine (902 μL, 8.19 mmol) in N,N-dimethylformamide:acetonitrile (30 mL, 1:1) was stirred at room temperature for 1 h.

Hydrazine hydrate (397 μL, 8.19 mmol) was added and the solution was stirred at room temperature for 16 h. The reaction mixture was evaporated and the residue was taken up in ethyl acetate (40 mL) and 10% aqueous sodium hydroxide (20 mL). The layers were separated and the organic layer was dried over sodium sulfate and evaporated to give the title compound (988 mg, 3.13 mmol, 76%) as an off-white powder. LCMS: 93%, Rt 1.551, ESMS m/z 316 (M+H)⁺.

Step 4. Compound 9d-1. 9-Benzyl-3-[1,3,4]oxadiazol-2-yl-carbazole. To a suspension of 9-benzylcarbazole-3-carboxylic acid hydrazide (Compound 9c-1, 988 mg, 3.13 mmol) in absolute ethanol (15 mL) under argon was added triethyl orthoformate (13 mL, 78.3 mmol) followed by a catalytic amount of p-toluenesulfonic acid monohydrate. The reaction mixture was heated to reflux for 3 h. The resulting precipitate was collected to give the title compound (793 mg, 2.44 mmol, 77%) as a white crystalline solid. LCMS: 99%, Rt 1.811, ESMS m/z 326 (M+H)⁺.

Step 5. Compound 9e-1. 3-[1,3,4]Oxadiazol-2-yl-9H-carbazole. To a solution of 9-benzyl-3-[1,3,4]oxadiazol-2-ylcarbazole (Compound 9d-1, 300 mg, 0.923 mmol) in benzene (35 mL) was added aluminum chloride (676 mg, 5.06 mmol) and the reaction mixture was stirred at room temperature for 16 h. The mixture was evaporated and the residue purified by column chromatography eluting with dichloromethane:ethyl acetate (98:2) to give the title compound (140 mg, 0.596 mmol, 64%) as an off-white powder. LCMS: 91%, Rt 1.462, ESMS m/z 236 (M+H)⁺;

Step 6. Compound 9-1. 9-(3-Fluoropropyl)-3-[1,3,4]oxadiazol-2-yl-carbazole. To a solution of 3-[1,3,4]oxadiazol-2-yl-9H-carbazole (Compound 9e-1, 70 mg, 0.298 mmol) in N,N-dimethylformamide:acetonitrile (1 mL, 1:1) was added cesium carbonate (189 mg, 0.596 mmol) and the mixture stirred at room temperature for 15 min. 1-Iodo-3-fluoropropane (36 μL, 0.35 mmol) was added and the mixture was stirred at room temperature for 2 h. The reaction mixture was evaporated and the residue was partitioned between chloroform (5 mL) and water (5 mL). The layers were separated and the organic layer was dried over sodium sulfate and evaporated. The residue was triturated with diethyl ether (1 mL) to give the title compound (45 mg, 0.152 mmol, 53%) as an off-white powder. LCMS: 98%, Rt 1.716, ESMS m/z 296 (M+H)⁺; ¹H NMR (500 MHz, CDCl₃) δ ppm 8.84 (s, 1H), 8.48 (s, 1H), 8.14-8.24 (m, 2H), 7.52-7.59 (m, 2H), 7.50 (d, J=8.3 Hz, 1H), 7.34 (t, J=7.3 Hz, 1H), 4.54 (t, J=6.6 Hz, 2H), 4.45 (dt, J=47.0, 5.4 Hz, 2H), 2.30 (dquint, J=27.9, 5.4 Hz, 2H).

### Example 10

### Compound 10-1. 5-(3-Fluoropropyl)-8-[1,3,4]oxadiazol-2-yl-5H-pyrido[3,2-b]indole

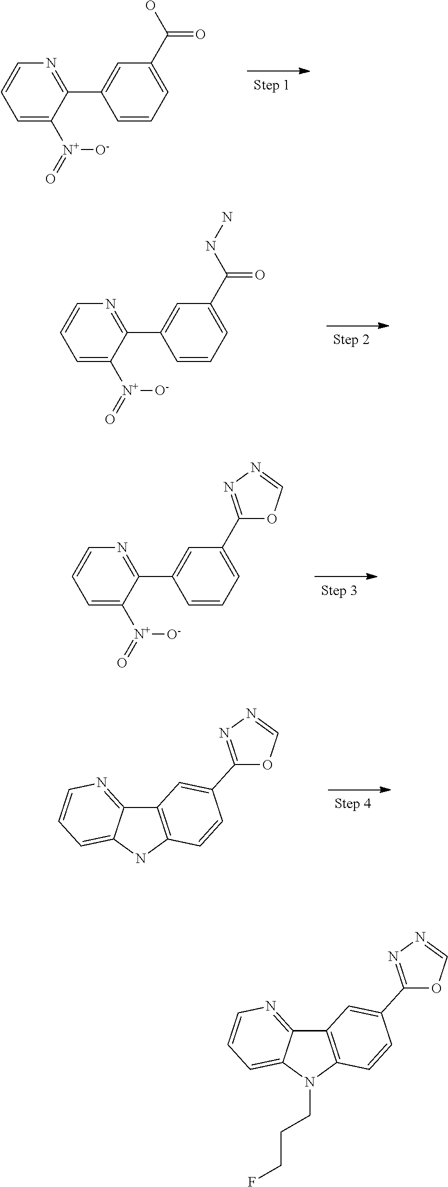 [SMILES: O=C(O)c1cccc(-c2ncccc2[N+](=O)[O-])c1 ; NNC(=O)c1cccc(-c2ncccc2[N+](=O)[O-])c1 ; O=[N+]([O-])c1cccnc1-c1cccc(-c2nnco2)c1 ; c1cnc2c(c1)[nH]c1ccc(-c3nnco3)cc12 ; FCCCn1c2ccc(-c3nnco3)cc2c2ncccc21]

Step 1. Compound 10a-1. 3-(3-Nitropyridin-2-yl)benzoic acid hydrazide. A mixture of 3-(3-nitropyridin-2-yl)benzoic acid (Compound 5a-14, 1.05 g, 4.30 mmol), N,N,N′,N′-tetramethyl-O-(7-azabenzotriazol-1-yl)uronium hexafluorophosphate (HATU; 1.96 g, 5.16 mmol) and N-methylmorpholine (948 μL, 8.60 mmol) in N,N-dimethylformamide:acetonitrile (30 mL, 1:2) was stirred at room temperature for 1 h. Hydrazine hydrate (417 μL, 8.60 mmol) was added and the solution was stirred at room temperature for 1 h. The reaction mixture was evaporated and the residue was taken up in 10% aqueous sodium hydroxide (20 mL) and ethyl acetate (20 mL). The mixture was stirred at 0° C. for 5 min and filtered. The filtrate layers were separated and the aqueous layer washed with ethyl acetate (2×30 mL). The combined organic layers were dried over sodium sulfate and evaporated to give the crude title compound (1.75 g, 6.78 mmol, 100%) as an off-white solid. LCMS: 58%, ESMS m/z 259 (M+H)⁺.

Step 2. Compound 10b-1. 3-Nitro-2-(3-[1,3,4]oxadiazol-2-ylphenyl)pyridine. To a suspension of 3-(3-nitropyridin-2-yl)benzoic acid hydrazide (Compound 10a-1, 800 mg, 1.86 mmol) in absolute ethanol (10 mL) was added triethyl orthoformate (6.14 mL, 46.5 mmol) followed by a catalytic amount of p-toluenesulfonic acid monohydrate and the reaction mixture was heated to reflux for 6 h. The mixture was evaporated and the crude product was purified by column chromatography eluting with dichloromethane:methanol (99:1→95:5) to give the title compound (783 mg, 2.92 mmol, 94%) as an off-white solid. LCMS: 82%, ESMS m/z 269 (M+H)⁺.

Step 3. Compound 10c-1. 8-[1,3,4]Oxadiazol-2-yl-5H-pyrido[3,2-b]indole. A mixture of 3-nitro-2-(3-[1,3,4]oxadiazol-2-yl-phenyl)pyridine (Compound 10b-1, 734 mg, 2.73 mmol) and triphenylphosphine (1.79 g, 6.84 mmol) in chlorobenzene (12 mL) was heated at 200° C. for 90 min by microwave irradiation under nitrogen. The mixture was evaporated and the residue was purified by column chromatography eluting with dichloromethane to give the title compound (160 mg, 0.68 mmol, 25%) as an off-white powder. LCMS: 83%, ESMS m/z 237 (M+H)⁺.

Step 4. Compound 10-1. 5-(3-Fluoropropyl)-8-[1,3,4]oxadiazol-2-yl-5H-pyrido[3,2-b]indole. To a suspension of 8-[1,3,4]oxadiazol-2-yl-5H-pyrido[3,2-b]indole (Compound 10c-1, 40 mg, 0.17 mmol) and cesium carbonate (110 mg, 0.34 mmol) in N,N-dimethylformamide (1 mL) was added 1-iodo-3-fluoropropane (40 mg, 22 μL, 0.25 mmol) dropwise and the mixture was stirred at room temperature for 1 h. The mixture was evaporated and the crude product was purified by column chromatography eluting with ethyl acetate to give the title compound (34 mg, 0.12 mmol, 68%) as an off-white powder. LCMS: 100%, Rt 1.219, ESMS m/z 297 (M+H)⁺; ¹H NMR (300 MHz, CDCl₃) 6 ppm 9.07 (d, J=0.9 Hz, 1H), 8.65 (d, J=4.7 Hz, 1H), 8.50 (s, 1H), 8.39 (dd, J=8.7, 1.7 Hz, 1H), 7.81 (d, J=8.5 Hz, 1H), 7.61 (d, J=8.7 Hz, 1H), 7.45 (dd, J=8.3, 4.7 Hz, 1H), 4.55 (t, J=6.7 Hz, 2H), 4.43 (dt, J=46.9, 6.0 Hz, 2H), 2.29 (dquint, J=27.7, 6.0 Hz, 2H).

Compound 10-2 listed in the table below was prepared in a similar manner.

| | | | | | Anal. | |
|---|---|---|---|---|---|---|
| | | | | | Meth- | |
| Ex. | Structure | MW | Ion | Rt | od | Yield |
| 10-2 | 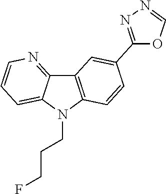 [SMILES: FCCCn1c2ccc(-c3nnco3)cc2c2ncccc21] | 296 | 297 | 1.219 | A | 68 |

### Example 11

### Compound 11-1. 9-(3-Fluoropropyl)-3-oxazol-5-yl-9H-carbazole

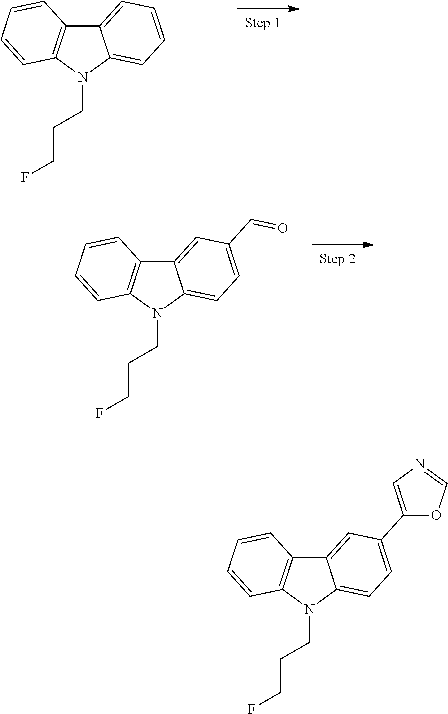 [SMILES: FCCCn1c2ccccc2c2ccccc21 ; O=Cc1ccc2c(c1)c1ccccc1n2CCCF ; FCCCn1c2ccccc2c2cc(-c3cnco3)ccc21]

Step 1. Compound 11a-1. 9-(3-Fluoropropyl)-9H-carbazole-3-carbaldehyde. To N,N-dimethylformamide (200 μL) at 0° C. was added phosphorus oxychloride (164 μL, 1.76 mmol) dropwise. The mixture was allowed to warm to room temperature and a solution of 9-(3-fluoropropyl)-9H-carbazole (1-4, 200 mg, 0.88 mmol) in N,N-dimethylformamide (800 μL) was added. The mixture was stirred at 80° C. for 16 h. The mixture was cooled to 0° C. and a solution of phosphorus oxychloride (164 μL, 1.76 mmol) in N,N-dimethylformamide (200 μL) was added. The mixture was stirred at 80° C. for 1 h. The reaction mixture was poured into ice water (2 mL) and 10% aqueous potassium hydroxide was added to achieve pH 8. The aqueous layer was extracted with dichloromethane (3×5 mL) and the combined organic layers were dried over sodium sulfate and evaporated. The residue was purified by column chromatography eluting with hexane:ethyl acetate (9:1) to give the title compound (132 mg, 0.515 mmol, 65%) as an off-white powder. LCMS: 95%, Rt 1.721, ESMS m/z 256 (M+H)⁺.

Step 2. Compound 11-1. 9-(3-Fluoropropyl)-3-oxazol-5-yl-9H-carbazole. To a mixture of 9-(3-fluoropropyl)-9H-carbazole-3-carbaldehyde (Compound 11a-1, 50 mg, 0.20 mmol) and p-toluenesulfonylmethyl isocyanide (42 mg, 0.22) in methanol (1 mL) was added potassium carbonate (54 mg, 0.39 mmol) and the mixture was heated at reflux for 3.5 h. The mixture was evaporated and the residue was partitioned between chloroform (6 mL) and water (4 mL). The layers were separated and the organic layer was dried over sodium sulfate and evaporated. The residue was purified by column chromatography eluting with hexane:ethyl acetate (6:1). The product was triturated with hexane (1.5 mL) to give the title compound (30 mg, 0.102 mmol, 52%) as a white powder. LCMS: 100%, Rt 1.808, ESMS m/z 295 (M+H)⁺; ¹H NMR (500 MHz, CDCl₃) δ ppm 8.40 (d, J=2.0 Hz, 1H), 8.16 (d, J=7.3 Hz, 1H), 7.95 (s, 1H), 7.78 (dd, J=8.6, 1.7 Hz, 1H), 7.51-7.55 (m, 1H), 7.45-7.51 (m, 2H), 7.38 (s, 1H), 7.30 (t, J=7.6 Hz, 1H), 4.52 (t, J=6.6 Hz, 2H), 4.44 (dt, J=47.0, 5.4 Hz, 2H), 2.28 (dquint, J=27.9, 5.4 Hz, 2H).

Compound 11-2 listed in the table below was prepared in a similar manner.

| | | | | | Anal. | |
|---|---|---|---|---|---|---|
| | | | | | Meth- | |
| Ex. | Structure | MW | Ion | Rt | od | Yield |
| 11-2 | 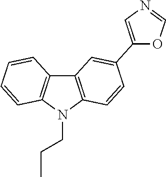 [SMILES: CCCn1c2ccccc2c2cc(-c3cnco3)ccc21] | 276 | 277 | 1.892 | A | 52 |

### Example 12

### Compound 12-1. 3-Oxazol-4-yl-9-propyl-9H-carbazole

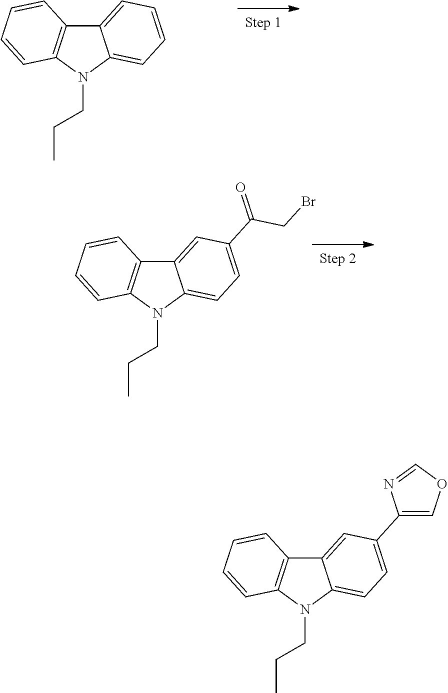 [SMILES: CCCn1c2ccccc2c2ccccc21 ; CCCn1c2ccccc2c2cc(C(=O)CBr)ccc21 ; CCCn1c2ccccc2c2cc(-c3cocn3)ccc21]

Step 1. Compound 12a-1. 2-Bromo-1-(9-propyl-9H-carbazol-3-yl)-ethanone. To a mixture of 9-propyl-9H-carbazole (Compound 1-5, 500 mg, 2.29 mmol) and aluminum chloride (350 mg, 2.63 mmol) in dichloroethane (7.15 mL) was added a solution of bromoacetyl chloride (328 μL, 3.94 mmol) in dichloroethane (3.90 mL) dropwise and the reaction mixture was stirred at room temperature for 40 h. The mixture was poured onto a mixture of 3 N hydrochloric acid and crushed ice (15 mL, 1:1) and extracted with dichloromethane (3×25 mL). The organic layer was washed with 1 M aqueous sodium carbonate, dried over sodium sulfate and evaporated. The residue was purified by column chromatography eluting with hexane:ethyl acetate (98:2-85:15) to give the title compound (517 mg, 1.56 mmol, 65%) as a pale brown powder. LCMS: 89%, Rt 1.889, ESMS m/z 330 (M+H)⁺.

Step 2. Compound 12-1. 3-Oxazol-4-yl-9-propyl-9H-carbazole. A solution of 2-bromo-1-(9-propyl-9H-carbazol-3-yl)ethanone (Compound 12a-1, 200 mg, 0.61 mmol) in formamide (3 mL) was stirred at 100° C. for 20 h. The reaction mixture was diluted with ethyl acetate (20 mL), washed with saturated sodium bicarbonate (20 mL) and water (20 mL), dried over sodium sulfate and evaporated. The residue was purified by column chromatography eluting with hexane:ethyl acetate (98:2-85:15) to give the title compound (35 mg, 0.127 mmol, 19%) as a pale yellow powder. LCMS: 99%, Rt 1.867, ESMS m/z 277 (M+H)⁺; ¹H NMR (300 MHz, DMSO-d₆) δ ppm 8.60 (s, 1H), 8.58 (s, 1H), 8.47 (s, 1H), 8.19 (d, J=7.7 Hz, 1H), 7.89 (d, J=8.5 Hz, 1H), 7.55-7.74 (m, 2H), 7.46 (t, J=7.6 Hz, 1H), 7.22 (t, J=7.4 Hz, 1H), 4.38 (t, J=6.9 Hz, 2H), 1.82 (sext, J=7.4 Hz, 2H), 0.88 (t, J=7.3 Hz, 3H).

### Example 13

### Compound 13-1. 3-Isoxazol-5-yl-9-propyl-9H-carbazole

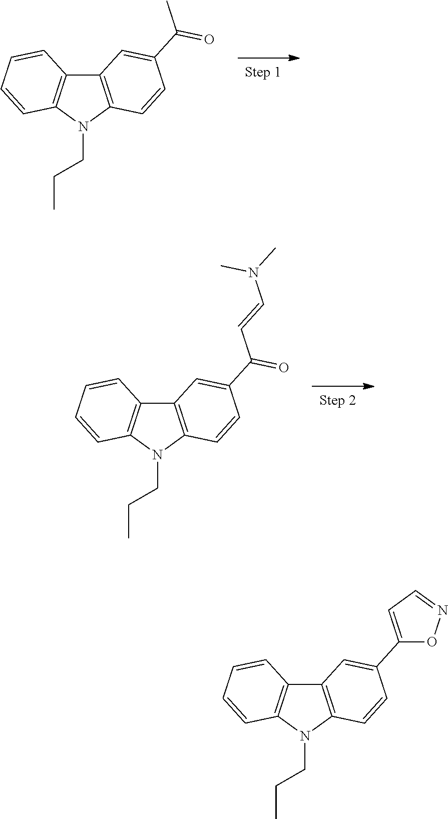 [SMILES: CCCn1c2ccccc2c2cc(C(C)=O)ccc21 ; CCCn1c2ccccc2c2cc(C(=O)/C=C/N(C)C)ccc21 ; CCCn1c2ccccc2c2cc(-c3ccno3)ccc21]

Step 1. Compound 13a-1. 3-Dimethylamino-1-(9-propyl-9H-carbazol-3-yl)propenone. A mixture of 1-(9-propyl-9H-carbazol-3-yl)ethanone (Compound 1-3, 300 mg, 1.19 mmol) in N,N-dimethylformamide dimethyl acetal (6 mL) was stirred at 105° C. for 3 d. The mixture was heated at 150° C. for 1 h under microwave irradiation. The reaction mixture was evaporated and the residue was purified by column chromatography eluting with chloroform:hexane (5:1) to give the title compound (198 mg, 0.646 mmol, 54%) as an off-white powder. LCMS: 57%, Rt 1.722, ESMS m/z 307 (M+H)⁺.

Step 2. Compound 13-1. 3-Isoxazol-5-yl-9-propyl-9H-carbazole. A mixture of 3-dimethylamino-1-(9-propyl-9H-carbazol-3-yl)propenone (Compound 13a-1, 80 mg, 0.261 mmol) and hydroxylamine hydrochloride (36 mg, 0.522 mmol) in ethanol (2 mL) was stirred at 80° C. for 16 h. The mixture was filtered and concentrated. The residue was purified by column chromatography eluting with hexane:ethyl acetate (20:1) to give the title compound (48 mg, 0.17 mmol, 66%) as a pale yellow oil. LCMS: 100%, Rt 1.922, ESMS m/z 277 (M+H)⁺; ¹H NMR (500 MHz, CDCl₃) δ ppm 8.56 (d, J=1.5 Hz, 1H), 8.31 (d, J=1.5 Hz, 1H), 8.16 (d, J=7.8 Hz, 1H), 7.90 (dd, J=8.6, 1.7 Hz, 1H), 7.50-7.54 (m, 1H), 7.48 (d, J=8.8 Hz, 1H), 7.45 (d, J=8.3 Hz, 1H), 7.30 (t, J=7.6 Hz, 1H), 6.54 (d, J=2.0 Hz, 1H), 4.32 (t, J=7.1 Hz, 2H), 1.95 (sext, J=7.3 Hz, 2H), 1.00 (t, J=7.4 Hz, 3H).

Compound 13-2 listed in the table below was prepared in a similar manner.

| | | | | | Anal. | |
|---|---|---|---|---|---|---|
| | | | | | Meth- | |
| Ex. | Structure | MW | Ion | Rt | od | Yield |
| 13-2 | 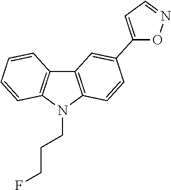 [SMILES: FCCCn1c2ccccc2c2cc(-c3ccno3)ccc21] | 294 | 295 | 1.875 | A | 40 |

### Example 14

### Compound 14-1. 9-(3-Fluoropropyl)-3-[1,2,4]triazol-1-yl-9H-carbazole

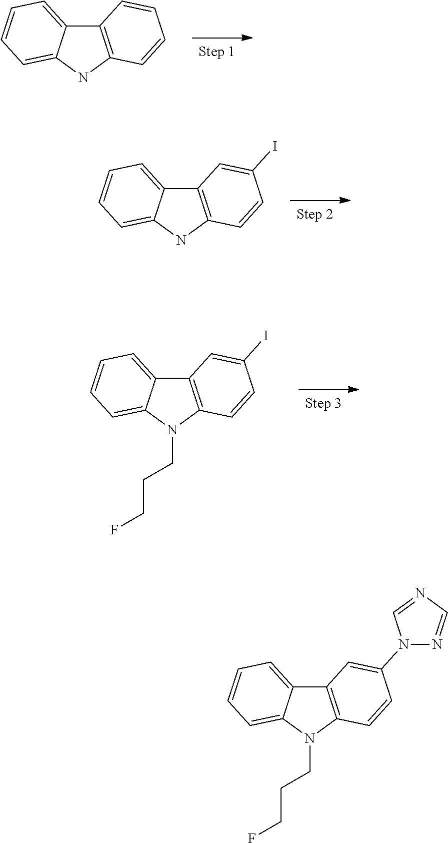 [SMILES: c1ccc2c(c1)[nH]c1ccccc12 ; Ic1ccc2[nH]c3ccccc3c2c1 ; FCCCn1c2ccccc2c2cc(I)ccc21 ; FCCCn1c2ccccc2c2cc(-n3cncn3)ccc21]

Step 1. Compound 14a-1. 3-Iodo-9H-carbazole. To a solution of 9H-carbazole (500 mg, 2.99 mmol) in acetic acid (15 mL) was added N-iodosuccinimide (740 mg, 3.29 mmol) and the reaction mixture was stirred at room temperature for 1 h. The mixture was evaporated and the residue was neutralized by addition of 10% aqueous potassium carbonate, and the resulting solid was collected. The crude product was purified by column chromatography eluting with hexane:ethyl acetate (33:1) to give the title compound (750 mg, 2.56 mmol, 86%) as a pale yellow oil. LCMS: 81%, Rt 1.900, ESMS m/z 292 (M−H)⁻.

Step 2. Compound 14b-1. 9-(3-Fluoropropyl)-3-iodo-9H-carbazole. To a solution of 3-iodo-9H-carbazole (Compound 14a-1, 100 mg, 0.341 mmol) and cesium carbonate (222 mg, 0.682 mmol) in N,N-dimethylformamide (2 mL) was added 1-iodo-3-fluoropropane (45 μL, 0.444 mmol) and the mixture stirred at room temperature for 2 h. The mixture was evaporated and the residue was diluted with water (3 mL) and extracted with chloroform (3×4 mL). The combined organic layers were dried over sodium sulfate and evaporated to give the title compound (112 mg, 0.32 mmol, 93%) as a colorless oil. LCMS: 95%, Rt 2.111, ESMS m/z 354 (M+H)⁺.

Step 3. Compound 14-1. 9-(3-Fluoropropyl)-3-[1,2,4]triazol-1-yl-9H-carbazole. A mixture of 9-(3-fluoropropyl)-3-iodo-9H-carbazole (Compound 14b-1, 110 mg, 0.311 mmol), 1,2,4-triazole (32 mg, 0.467 mmol), potassium phosphate (132 mg, 0.623 mmol), N,N′-dimethylethylenediamine (57 μL, 0.529 mmol) and copper(I) iodide (6 mg, 0.031 mmol) in N,N-dimethylformamide (2 mL) was stirred at 100° C. for 20 h. The reaction mixture was evaporated and the residue was purified by column chromatography eluting with dichloromethane. The product was triturated with hexane (0.5 mL) to give the title compound (15 mg, 0.051 mmol, 16%) as a white powder. LCMS: 100%, Rt 1.621, ESMS m/z 295 (M+H)⁺; ¹H NMR (500 MHz, CDCl₃) δ ppm 8.40 (d, J=2.0 Hz, 1H), 8.16 (d, J=7.3 Hz, 1H), 7.95 (s, 1H), 7.78 (dd, J=8.6, 1.7 Hz, 1H), 7.51-7.55 (m, 1H), 7.45-7.51 (m, 2H), 7.38 (s, 1H), 7.30 (t, J=7.6 Hz, 1H), 4.52 (t, J=6.6 Hz, 2H), 4.44 (dt, J=47.0, 5.4 Hz, 2H), 2.19 (dquint, J=26.9, 5.4 Hz, 2H).

### Example 15

### Compound 15-1. 3-(4,4-Dimethyl-4,5-dihydrooxazol-2-yl)-9-propyl-9H-carbazole

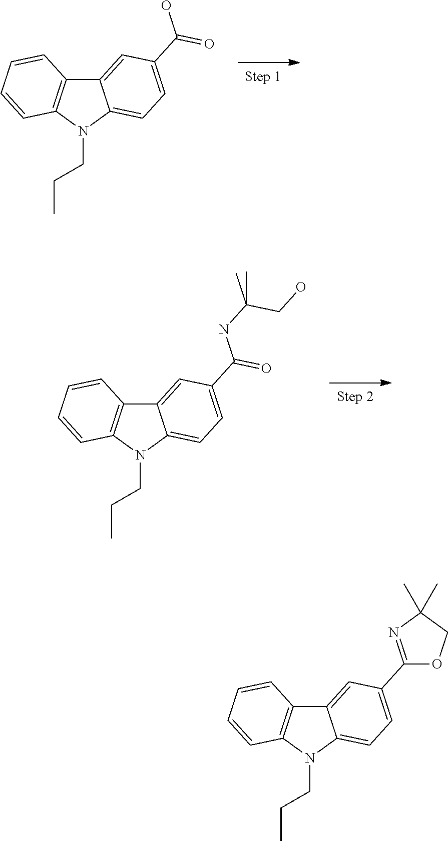 [SMILES: CCCn1c2ccccc2c2cc(C(=O)O)ccc21 ; CCCn1c2ccccc2c2cc(C(=O)NC(C)(C)CO)ccc21 ; CCCn1c2ccccc2c2cc(C3=NC(C)(C)CO3)ccc21]

Step 1. Compound 15a-1. 9-Propyl-9H-carbazole-3-carboxylic acid (2-hydroxy-1,1-dimethylethyl)-amide. A mixture of 9-propyl-9H-carbazole-3-carboxylic acid (Compound 8a-1, 200 mg, 0.79 mmol), 2-amino-2-methyl-1-propanol (220 μL, 2.33 mmol), N-(3-dimethylaminopropyl)-N′-ethylcarbodiimide hydrochloride (EDC, 447 mg, 2.33 mmol) and 1-hydroxybenzotriazole (315 mg, 2.33 mmol) in N,N-dimethylformamide (5 mL) was stirred at room temperature for 16 h. The mixture was evaporated, taken up in chloroform (7 mL) and washed with 5% aqueous sodium bicarbonate (5 mL). The organic layer was dried over sodium sulfate and evaporated to give the title compound as a yellow oil. The crude product was used in the next step without further purification. LCMS: 90%, Rt 1.670, ESMS m/z 325 (M+H)+.

Compound 15a-2 listed in the table below was prepared in a similar manner.

| | | | | | Anal. | |
|---|---|---|---|---|---|---|
| | | | | | Meth- | |
| Ex. | Structure | MW | Ion | Rt | od | Yield |
| 15a-2 | 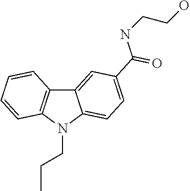 [SMILES: CCCn1c2ccccc2c2cc(C(=O)NCCO)ccc21] | 296 | 297 | 1.439 | A | Quant (crude) |

Step 2. Compound 15-1. 3-(4,4-Dimethyl-4,5-dihydrooxazol-2-yl)-9-propyl-9H-carbazole. To a solution of 9-propyl-9H-carbazole-3-carboxylic acid (2-hydroxy-1,1-dimethylethyl)-amide (Compound 15a-1, 200 mg, 0.62 mmol) in ethyl acetate (5 mL) was added thionyl chloride (67 μL, 0.93 mmol) dropwise and the mixture was stirred at room temperature for 2 h. The reaction was evaporated and the residue was dissolved in chloroform (10 mL), washed with 10% aqueous sodium hydroxide (2×5 mL), dried over sodium sulfate and evaporated. The residue was purified by column chromatography eluting with hexane:ethyl acetate (10:1). The product was triturated with hexane (1 mL) to give the title compound (43 mg, 0.14 mmol, 23%) as a white powder. LCMS: 100%, Rt 1.664, ESMS m/z 307 (M+H)⁺; ¹H NMR (500 MHz, CDCl₃) δ ppm 8.72 (d, J=1.7 Hz, 1H), 8.14 (d, J=7.8 Hz, 1H), 8.08 (dd, J=8.6, 1.7 Hz, 1H), 7.46-7.52 (m, 1H), 7.38-7.46 (m, 2H), 7.24-7.29 (m, 1H), 4.30 (t, J=7.3 Hz, 2H), 4.17 (s, 2H), 1.93 (sext, J=7.3 Hz, 2H), 1.45 (s, 6H), 0.98 (t, J=7.4 Hz, 3H).

Compound 15-2 listed in the table below was prepared in a similar manner.

| | | | | | Anal. | |
|---|---|---|---|---|---|---|
| | | | | | Meth- | |
| Ex. | Structure | MW | Ion | Rt | od | Yield |
| 15-2 | 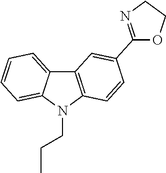 [SMILES: CCCn1c2ccccc2c2cc(C3=NCCO3)ccc21] | 278 | 279 | 1.479 | A | 6 |

### Example 16

### Compound 16-1. 1-[9-(2-Fluoropropyl)-9H-carbazol-3-yl]-ethanone

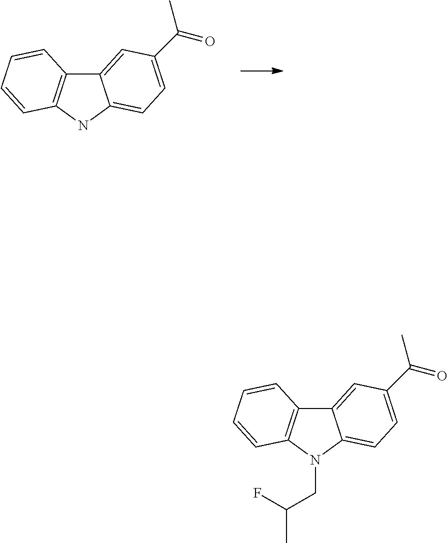 [SMILES: CC(=O)c1ccc2[nH]c3ccccc3c2c1 ; CC(=O)c1ccc2c(c1)c1ccccc1n2CC(C)F]

A mixture of 3-acetyl-9H-carbazole (100 mg, 0.48 mmol), tri-n-butylphosphine (194 mg, 0.96 mmol), N,N,N′,N′-tetramethylazodicarboxamide (165 mg, 0.96 mmol) and 2-fluoropropan-1-ol (75 mg, 0.96 mmol) in toluene (3 mL) was stirred at 40° C. for 3 d. The reaction mixture was evaporated, taken up in chloroform (10 mL) and washed with water (2×5 mL). The organic layer was dried over sodium sulfate and concentrated. The resulting yellow oil was purified by column chromatography eluting with hexane:ethyl acetate (10:1). The solid was triturated with hexane:diethyl ether (5:1, 1 mL) to give the title compound (19 mg, 0.07 mmol, 15%) as a white powder. LCMS: 100%, Rt 1.648, ESMS m/z 270 (M+H)⁺; ¹H NMR (500 MHz, CDCl₃) δ ppm 8.76 (d, J=1.0 Hz, 1H), 8.17 (d, J=7.8 Hz, 1H), 8.14 (dd, J=8.6, 1.7 Hz, 1H), 7.53 (t, J=7.3 Hz, 1H), 7.43-7.49 (m, 2H), 7.34 (t, J=7.3 Hz, 1H), 5.01-5.24 (dm, J=47.9 Hz, 1H), 4.40-4.58 (m, 2H), 2.74 (s, 3H), 1.46 (dd, J=23.5, 6.4 Hz, 3H).

### Example 17

### Compounds 17-1 1-[9-(3-Fluoropropyl)-9H-beta-carbolin-3-yl]-ethanone and 17-2 2-[9-(3-fluoropropyl)-9H-beta-carbolin-3-yl]-propan-2-ol

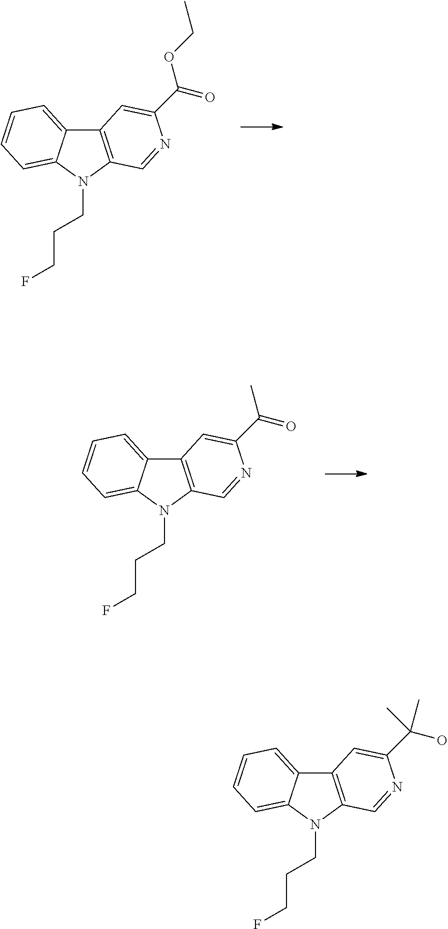 [SMILES: CCOC(=O)c1cc2c3ccccc3n(CCCF)c2cn1 ; CC(=O)c1cc2c3ccccc3n(CCCF)c2cn1 ; CC(C)(O)c1cc2c3ccccc3n(CCCF)c2cn1]

To a solution of 9-(3-fluoropropyl)-9H-beta-carboline-3-carboxylic acid ethyl ester (Compound 1-6, 200 mg, 0.67 mmol) in tetrahydrofuran (4 mL) at −30° C. was added methylmagnesium bromide (3 M solution in tetrahydrofuran, 222 μL, 0.67 mmol) and the mixture stirred at −30° C. for 1 h. The mixture was then stirred at room temperature for 1 h. The mixture was evaporated, diluted with water (10 mL) and extracted with chloroform (2×10 mL). The combined organic layers were dried over sodium sulfate and evaporated. The crude product was purified by column chromatography eluting with hexane:ethyl acetate (4:1) to give 1-[9-(3-fluoropropyl)-9H-beta-carbolin-3-yl]-ethanone (Compound 17-1, 20 mg, 0.07 mmol, 11%) as a white powder: LCMS: 100%, Rt 1.348, ESMS m/z 271 (M+H)⁺; ¹H NMR (500 MHz, DMSO-d₆) δ ppm 9.13 (s, 1H), 8.85 (s, 1H), 8.45 (d, J=7.8 Hz, 1H), 7.78 (d, J=8.3 Hz, 1H), 7.65-7.71 (m, 1H), 7.37 (t, J=7.6 Hz, 1H), 4.69 (t, J=6.8 Hz, 2H), 4.44 (dt, J=47.2, 5.9 Hz, 2H), 2.73 (s, 3H), 2.24 (dquint, J=27.4, 5.9 Hz, 2H) and 2-[9-(3-fluoropropyl)-9H-beta-carbolin-3-yl]-propan-2-ol (Compound 17-2, 16 mg, 0.06 mmol, 8%) as an off-white powder: LCMS: 98%, Rt 1.064, ESMS m/z 287 (M+H)⁺; ¹H NMR (500 MHz, DMSO-d₆) δ ppm 8.91 (s, 1H), 8.35 (s, 1H), 8.27 (d, J=7.3 Hz, 1H), 7.67 (d, J=7.6 Hz, 1H), 7.60 (t, J=7.6 Hz, 1H), 7.27 (t, J=7.3 Hz, 1H), 5.23 (s, 1H), 4.58 (t, J=6.8 Hz, 2H), 4.43 (dt, J=47.4, 5.9 Hz, 2H), 2.21 (dquint, J=26.9, 5.9 Hz, 2H), 1.54 (s, 6H).

Compounds 17-3-17-4 listed in the table below were prepared in a similar manner.

| | | | | | Anal. | |
|---|---|---|---|---|---|---|
| | | | | | Meth- | |
| Ex. | Structure | MW | Ion | Rt | od | Yield |
| 17-3 | 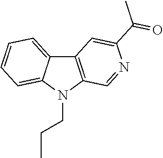 [SMILES: CCCn1c2ccccc2c2cc(C(C)=O)ncc21] | 252 | 253 | 1.397 | A | 13 |
| 17-4 | 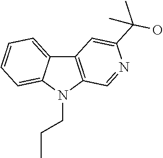 [SMILES: CCCn1c2ccccc2c2cc(C(C)(C)O)ncc21] | 268 | 269 | 1.200 | A | 29 |

### Example 18

### Compound 18-1, 1-[9-(3,3-Dichloropropyl)-9H-carbazol-3-yl]-ethanone and Compound 18-2, 1-[9-(3,3-Difluoropropyl)-9H-carbazol-3-yl]-ethanone

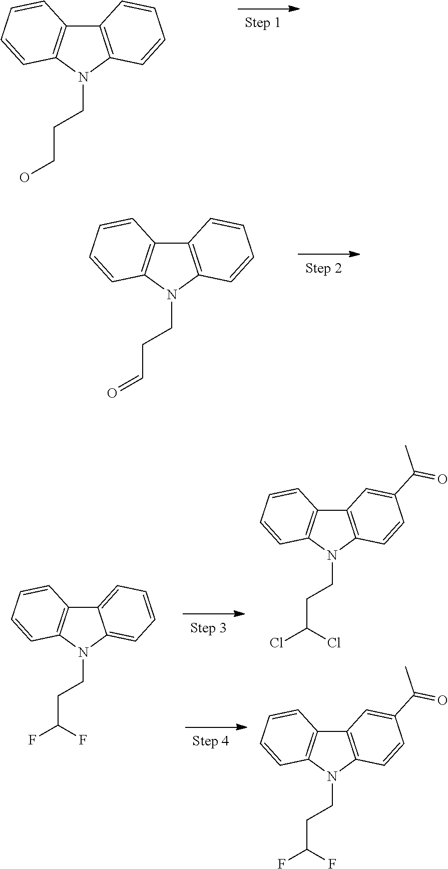 [SMILES: OCCCn1c2ccccc2c2ccccc21 ; O=CCCn1c2ccccc2c2ccccc21 ; FC(F)CCn1c2ccccc2c2ccccc21 ; CC(=O)c1ccc2c(c1)c1ccccc1n2CCC(Cl)Cl ; CC(=O)c1ccc2c(c1)c1ccccc1n2CCC(F)F]

Step 1. Compound 18a-1. 3-Carbazol-9-ylpropionaldehyde. A mixture of 3-carbazol-9-ylpropan-1-ol (Compound 1-8, 500 mg, 2.22 mmol) and N,N-dicyclohexylcarbodiimide (1.37 g, 6.66 mmol) in dimethyl sulfoxide (15 mL) was added to a solution of pyridine (178 μL, 2.22 mmol) and trifluoroacetic acid (85 μL, 1.11 mmol) in benzene (37 mL) and the reaction mixture stirred at room temperature for 20 h. Diethyl ether (12 mL) was added, followed by a solution of oxalic acid (96 mg, 8.88 mmol) in methanol (12 mL), and the mixture was stirred at room temperature for 15 min. The reaction was quenched with water (40 mL) and the mixture was filtered to remove 1,3-dicyclohexylurea. The layers were separated and the organic layer dried over sodium sulfate and evaporated. The residue was purified by column chromatography eluting with hexane:ethyl acetate (6:1) to give the title compound (373 mg, 1.67 mmol, 75%) as a white powder. ESMS m/z 224 (M+H)⁺.

Step 2. Compound 18b-1. 9-(3,3-Difluoropropyl)-9H-carbazole. A solution of 3-carbazol-9-ylpropionaldehyde (Compound 18a-1, 370 mg, 1.66 mmol) in dichloromethane (10 mL) was cooled to −20° C. Diethylaminosulfur trifluoride (210 μL, 1.66 mmol) was added and the reaction mixture stirred at −20° C. for 1 h. The mixture was evaporated and the residue purified by column chromatography eluting with hexane:ethyl acetate (20:1) to give the title compound (360 mg, 1.47 mmol, 88%) as a white powder. ESMS m/z 246 (M+H)⁺.

Step 3. Compound 18-1. 1-[9-(3,3-Dichloropropyl)-9H-carbazol-3-yl]-ethanone. To a solution of 9-(3,3-difluoropropyl)-9H-carbazole (Compound 18b-1, 100 mg, 0.408 mmol) in dichloromethane (1 mL) was added aluminum chloride (109 mg, 0.815 mmol). To the mixture was added a solution of acetyl chloride (29 μL, 0.408 mmol) in dichloromethane (1 mL) dropwise and the resulting mixture stirred at room temperature for 16 h. The reaction mixture was diluted with dichloromethane (10 mL) and washed with saturated sodium bicarbonate (10 mL). The aqueous layer was extracted with dichloromethane (2×5 mL) and the combined organic layers were dried over sodium sulfate and evaporated. The residue was purified by column chromatography eluting with hexane:ethyl acetate (20:1). The product was triturated with hexane (1 mL) to give the title compound (10 mg, 0.03 mmol, 7%) as a white powder. LCMS: 98%, Rt 1.972, ESMS m/z 320 (M+H)⁺; ¹H NMR (500 MHz, CDCl₃) δ ppm 8.77 (d, J=1.5 Hz, 1H), 8.12-8.21 (m, 2H), 7.53-7.58 (m, 1H), 7.46-7.52 (m, 2H), 7.35 (t, J=7.1 Hz, 1H), 5.75 (t, J=6.8 Hz, 1H), 4.65 (t, J=6.8 Hz, 2H), 2.78 (q, J=6.4 Hz, 2H), 2.74 (s, 3H).

Step 4. Compound 18-2. 1-[9-(3,3-Difluoropropyl)-9H-carbazol-3-yl]-ethanone. To a solution of 9-(3,3-difluoropropyl)-9H-carbazole (Compound 18b-1, 125 mg, 0.509 mmol) in dichloromethane (2.5 mL) was added aluminum chloride (68 mg, 0.509 mmol). To this mixture was added a solution of acetyl chloride (36 μL, 0.509 mmol) in dichloromethane (2.5 mL) dropwise. The mixture stirred at room temperature for 1 h. The reaction mixture was diluted with dichloromethane (10 mL) and washed with saturated sodium bicarbonate (10 mL). The aqueous layer was extracted with dichloromethane (2×5 mL) and the combined organic layers were dried over sodium sulfate and evaporated. The residue was purified by column chromatography eluting with hexane:ethyl acetate (20:1). The product was triturated with hexane (1.5 mL) to give the title compound (37 mg, 0.128 mmol, 25%) as a white powder. LCMS: 100%, Rt 1.785, ESMS m/z 288 (M+H)⁺; ¹H NMR (300 MHz, CDCl₃) 6 ppm 8.75 (s, 1H), 8.03-8.24 (m, 2H), 7.49-7.59 (m, 1H), 7.38-7.47 (m, 2H), 7.34 (t, J=7.4 Hz, 1H), 5.82 (t, J=55.2 Hz, 1H), 4.55 (t, J=6.9 Hz, 2H), 2.73 (s, 3H), 2.43 (m. 2.31-2.53 2H).

### Example 19

### Compound 19-1. 6-Acetyl-9-propyl-9H-carbazole-4-carboxamide

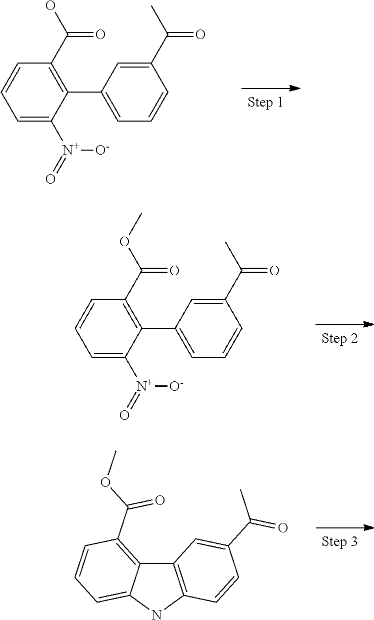

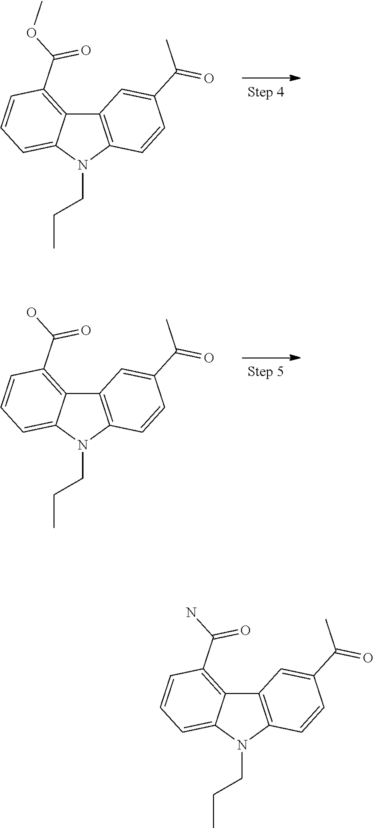 [SMILES: CCCn1c2ccc(C(C)=O)cc2c2c(C(=O)OC)cccc21 ; CCCn1c2ccc(C(C)=O)cc2c2c(C(=O)O)cccc21 ; CCCn1c2ccc(C(C)=O)cc2c2c(C(N)=O)cccc21]

Step 1. Compound 19a-1. 3′-Acetyl-6-nitrobiphenyl-2-carboxylic acid methyl ester. To 3′-acetyl-6-nitrobiphenyl-2-carboxylic acid (Compound 5a-6, 2.0 g, 7.0 mmol) was added a solution of hydrogen chloride in methanol (5.47 M, 15 mL) was stirred at 50° C. for 16 h. The solution was evaporated and the residue was taken up in chloroform (40 mL) and washed with saturated sodium bicarbonate (2×20 mL). The organic layer was dried over sodium sulfate and evaporated to give the title compound (1.82 g, 6.08 mmol, 86%) as a yellow powder. LCMS: 85%, Rt 1.556, ESMS m/z 300 (M+H)⁺.

Compound 19a-2 listed in the table below was prepared in a similar manner.

| | | | | | Anal. | |
|---|---|---|---|---|---|---|
| Ex. | Structure | MW | Ion | Rt | Method | Yield |
| 19a-2 | 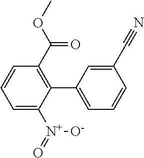 [SMILES: COC(=O)c1cccc([N+](=O)[O-])c1-c1cccc(C#N)c1] | 282 | 283 | 1.573 | A | 74 |

Step 2. Compound 19b-1. 6-Acetyl-9H-carbazole-4-carboxylic acid methyl ester. A mixture of 3′-acetyl-6-nitrobiphenyl-2-carboxylic acid methyl ester (Compound 19a-1, 1.80 g, 6.01 mmol) and 1,2-bis(diphenylphosphino)ethane (DPPE, 3.35 g, 8.41 mmol) was stirred at 140° C. for 6 h. The reaction mixture was purified by column chromatography eluting with hexane:ethyl acetate (3:1). The product was triturated with diethyl ether to give the title compound (395 mg contaminated with DPPEO), which was used in the next step without further purification. LCMS: 29%, Rt 1.510, ESMS m/z 268 (M+H)⁺.

Compound 19b-2 listed in the table below was prepared in a similar manner.

| | | | | | Anal. | |
|---|---|---|---|---|---|---|
| Ex. | Structure | MW | Ion | Rt | Method | Yield |
| 19b-2 | 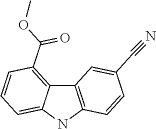 [SMILES: COC(=O)c1cccc2[nH]c3ccc(C#N)cc3c12] | 250 | 251 | 1.585 | A | crude |

Step 3. Compound 19c-1. 6-Acetyl-9-propyl-9H-carbazole-4-carboxylic acid methyl ester. To a suspension of 6-acetyl-9H-carbazole-4-carboxylic acid methyl ester (Compound 19b-1, 395 mg crude) and cesium carbonate (964 mg, 2.96 mmol) in N,N-dimethylformamide (8 mL) was added 1-bromopropane (135 μL, 1.48 mmol) and the mixture stirred at room temperature for 1 h. The mixture was evaporated and the residue was diluted with water (20 mL) and extracted with chloroform (2×20 mL). The combined organic layers were dried over sodium sulfate and evaporated. The resulting yellow solid (385 mg) was used in the next step without further purification. LCMS: 29%, Rt 1.858, ESMS m/z 310 (M+H)⁺.

Compound 19c-2 listed in the table below was prepared in a similar manner.

| | | | | | Anal. | |
|---|---|---|---|---|---|---|
| Ex. | Structure | MW | Ion | Rt | Method | Yield |
| 19c-2 | 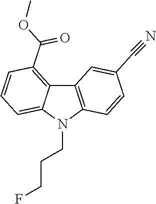 [SMILES: COC(=O)c1cccc2c1c1cc(C#N)ccc1n2CCCF] | 310 | 311 | 1.798 | A | crude |

Step 4. Compound 19d-1. 6-Acetyl-9-propyl-9H-carbazole-4-carboxylic acid. A mixture of 6-acetyl-9-propyl-9H-carbazole-4-carboxylic acid methyl ester (Compound 19c-1, 385 mg crude) in 1,4-dioxane and 20% aqueous sodium hydroxide (2:1, 9 mL) was heated to 50° C. for 16 h. The reaction mixture was concentrated and the residue was partitioned between 10% aqueous sodium hydroxide (15 mL) and chloroform (10 mL). The layers were separated and the aqueous layer was acidified to pH 4 with 10% hydrochloric acid and extracted with chloroform (3×20 mL). The combined organic layers were dried over sodium sulfate and evaporated to give the title compound (64 mg, 0.216 mmol, 4% over 3 steps) as a white powder. LCMS: 100%, Rt 1.585, ESMS m/z 296 (M+H)⁺; ¹H NMR (500 MHz, DMSO-d₆) 6 ppm 13.25 (br. s, 1H), 9.61 (d, J=1.5 Hz, 1H), 8.12 (dd, J=8.8, 2.0 Hz, 1H), 7.99 (d, J=7.8 Hz, 1H), 7.85 (d, J=7.3 Hz, 1H), 7.77 (d, J=8.8 Hz, 1H), 7.61 (t, J=8.1 Hz, 1H), 4.49 (t, J=7.1 Hz, 2H), 2.65 (s, 3H), 1.81 (sext, J=7.3 Hz, 2H), 0.88 (t, J=7.3 Hz, 3H).

Compound 19d-2 listed in the table below was prepared in a similar manner.

| | | | | | Anal. | |
|---|---|---|---|---|---|---|
| | | | | | Meth- | |
| Ex. | Structure | MW | Ion | Rt | od | Yield |
| 19d-2 | 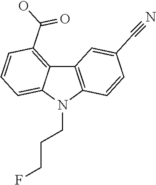 [SMILES: N#Cc1ccc2c(c1)c1c(C(=O)O)cccc1n2CCCF] | 296 | 297 | 1.525 | A | 4 (3 steps) |

Step 5. Compound 19-1. 6-Acetyl-9-propyl-9H-carbazole-4-carboxamide. A mixture of 6-acetyl-9-propyl-9H-carbazole-4-carboxylic acid (Compound 19d-1, 64 mg, 0.216 mmol), O-(7-azabenzotriazol-1-yl)-N,N,N′,N′-tetramethyluronium hexafluorophosphate (HATU, 165 mg, 0.43 mmol), N-methylmorpholine (71 μL, 0.65 mmol) in acetonitrile (2 mL) and N,N-dimethylformamide (1.5 mL) was stirred at room temperature for 30 min. To the reaction mixture was added a solution of ammonia in 1,4-dioxane (3 M, 3 mL) and the mixture stirred at room temperature for 16 h. The mixture was evaporated and the residue was diluted with ethyl acetate (15 mL) and washed with water (1×7 mL), 5% aqueous sodium hydroxide (1×7 mL) and brine (1×7 mL). The organic layer was dried over sodium sulfate and evaporated. The residue was triturated with diethyl ether (2 mL) and the product was recrystallized with ethyl acetate (1 mL) to give the title compound (18 mg, 0.061 mmol, 28%) as an off-white powder. Rt 1.432, ESMS m/z 295 (M+H)⁺; ¹H NMR (500 MHz, DMSO-d₆) 6 ppm 9.17 (d, J=1.0 Hz, 1H), 8.09 (dd, J=8.8, 1.5 Hz, 1H), 8.07 (br. s, 1H), 7.81 (d, J=8.3 Hz, 1H), 7.73 (d, J=8.3 Hz, 1H), 7.70 (br. s, 1H), 7.55 (t, J=7.8 Hz, 1H), 7.42 (d, J=7.3 Hz, 1H), 4.46 (t, J=7.1 Hz, 2H), 2.62 (s, 3H), 1.81 (sext, J=7.3 Hz, 2H), 0.87 (t, J=7.4 Hz, 3H).

Compound 19-2 listed in the table below was prepared in a similar manner.

| | | | | | Anal. | |
|---|---|---|---|---|---|---|
| Ex. | Structure | MW | Ion | Rt | Method | Yield |
| 19-2 | 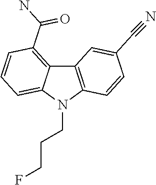 [SMILES: N#Cc1ccc2c(c1)c1c(C(N)=O)cccc1n2CCCF] | 295 | 296 | 1.314 | A | 7 |

### Example 20

### Compound 20-1. 1-(6-Bromo-9-propyl-9H-carbazol-3-yl)-ethanone

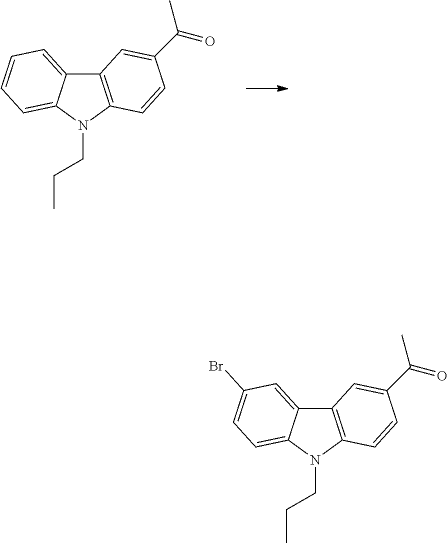 [SMILES: CCCn1c2ccccc2c2cc(C(C)=O)ccc21 ; CCCn1c2ccc(Br)cc2c2cc(C(C)=O)ccc21]

To a solution of 1-(9-propyl-9H-carbazol-3-yl)-ethanone (Compound 1-3, 20 mg, 0.079 mmol) in dichloroethane (500 μL) was added N-bromosuccinimide (14 mg, 0.079 mmol) and the mixture stirred at room temperature for 16 h. The mixture was evaporated and the residue was taken up in ethyl acetate (5 mL) and washed with water (3 mL). The organic layer was dried over sodium sulfate and concentrated. The residue was purified by column chromatography eluting with hexane:ethyl acetate (8:1) to give the title compound (12 mg, 0.036 mmol, 46%) as a white powder. LCMS: 98%, Rt 1.954, ESMS m/z 331 (M+H)⁺; ¹H NMR (500 MHz, CDCl₃) δ ppm 8.70 (s, 1H), 8.28 (s, 1H), 8.16 (d, J=8.8 Hz, 1H), 7.60 (d, J=8.8 Hz, 1H), 7.43 (d, J=8.8 Hz, 1H), 7.33 (d, J=8.8 Hz, 1H), 4.29 (t, J=6.8 Hz, 2H), 2.73 (s, 3H), 1.93 (sext, J=7.2 Hz, 2H), 0.98 (t, J=7.4 Hz, 3H).

Compound 20-2 listed in the table below was prepared in a similar manner.

| | | | | | Anal. | |
|---|---|---|---|---|---|---|
| | | | | | Meth- | |
| Ex. | Structure | MW | Ion | Rt | od | Yield |
| 20-2 | 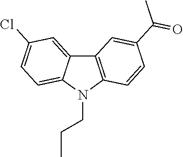 [SMILES: CCCn1c2ccc(Cl)cc2c2cc(C(C)=O)ccc21] | 286 | 286 | 1.951 | A | 46 |

### Example 21

### Compound 21-1. 1-(6-Methoxy-9-propyl-9H-carbazol-3-yl)-ethanone

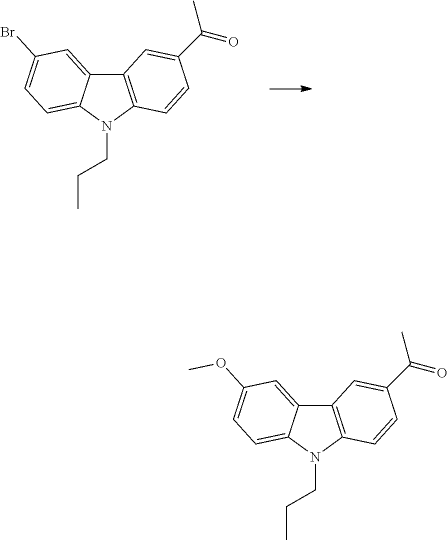 [SMILES: CCCn1c2ccc(Br)cc2c2cc(C(C)=O)ccc21 ; CCCn1c2ccc(OC)cc2c2cc(C(C)=O)ccc21]

To the solution of sodium (264 mg, 11.50 mmol) in methanol (3.8 mL) was added a mixture of copper(I) iodide (284 mg, 1.50 mmol) and 1-(6-bromo-9-propyl-9H-carbazol-3-yl)-ethanone (Compound 20-1, 190 mg, 0.575 mmol) in N,N-dimethylformamide (3.8 mL). The mixture was stirred at 130° C. for 3.5 h. The mixture was evaporated and the residue was purified by column chromatography eluting with hexane:ethyl acetate (5:1) to give the title compound (6 mg, 0.021 mmol, 4%) as a pale yellow powder. LCMS: 94%, Rt 1.766, ESMS m/z 282 (M+H)⁺; ¹H NMR (500 MHz, CDCl₃) δ ppm 8.72 (d, J=1.5 Hz, 1H), 8.11 (dd, J=8.8, 1.5 Hz, 1H), 7.65 (d, J=2.4 Hz, 1H), 7.38 (d, J=8.8 Hz, 1H), 7.35 (d, J=8.8 Hz, 1H), 7.16 (dd, J=8.8, 2.4 Hz, 1H), 4.27 (t, J=7.1 Hz, 2H), 3.96 (s, 3H), 2.73 (s, 3H), 1.83 (sext, J=7.3 Hz, 2H), 0.97 (t, J=7.4 Hz, 3H).

### Example 22

### Compound 22-1. 6-Acetyl-9-propyl-9H-carbazole-3-carbonitrile

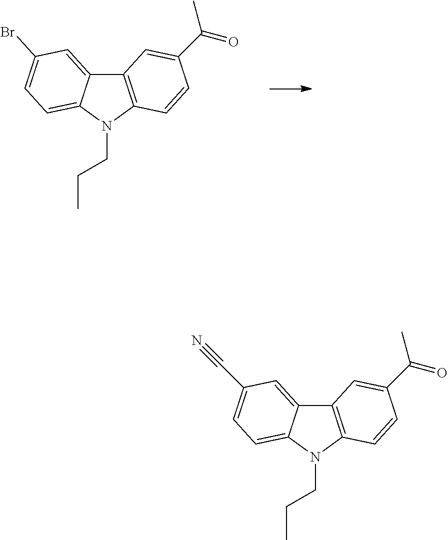 [SMILES: CCCn1c2ccc(Br)cc2c2cc(C(C)=O)ccc21 ; CCCn1c2ccc(C#N)cc2c2cc(C(C)=O)ccc21]

A mixture of 1-(6-bromo-9-propyl-9H-carbazol-3-yl)-ethanone (Compound 20-1, 200 mg, 0.606 mmol), potassium cyanide (79 mg, 1.21 mmol), 1,8-diazabicyclo[5.4.0]undec-7-ene (91 μL, 0.606 mmol) and tetrakis(triphenylphosphine) palladium(0) (70 mg, 0.061 mmol) in N-methylpyrrolidinone (4 mL) was heated at 150° C. for 1 h under microwave irradiation. The mixture was concentrated and residue was purified by column chromatography eluting with hexane:ethyl acetate (5:1). The product was triturated with hexane (2 mL) to give the title compound (14 mg, 0.051 mmol, 8%) as an off-white powder. LCMS: 100%, Rt 1.696, ESMS m/z 277 (M+H)⁺; ¹H NMR (500 MHz, CDCl₃) δ ppm 8.75 (d, J=1.6 Hz, 1H), 8.48 (d, J=1.1 Hz, 1H), 8.22 (dd, J=8.8, 1.6 Hz, 1H), 7.77 (dd, J=8.8, 1.6 Hz, 1H), 7.48-7.53 (m, 2H), 4.35 (t, J=7.1 Hz, 2H), 2.75 (s, 3H), 1.96 (sext, J=7.3 Hz, 2H), 1.00 (t, J=7.4 Hz, 3H)

Compound 22-2 listed in the table below was prepared in a similar manner.

| | | | | | Anal. | |
|---|---|---|---|---|---|---|
| Ex. | Structure | MW | Ion | Rt | Method | Yield |
| 22-2 | 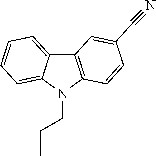 [SMILES: CCCn1c2ccccc2c2cc(C#N)ccc21] | 234 | 235 | 1.844 | A | 12 |

### Example 23

### Compound 23-1. 6-Acetyl-9-propyl-9H-carbazole-3-carboxylic acid amide

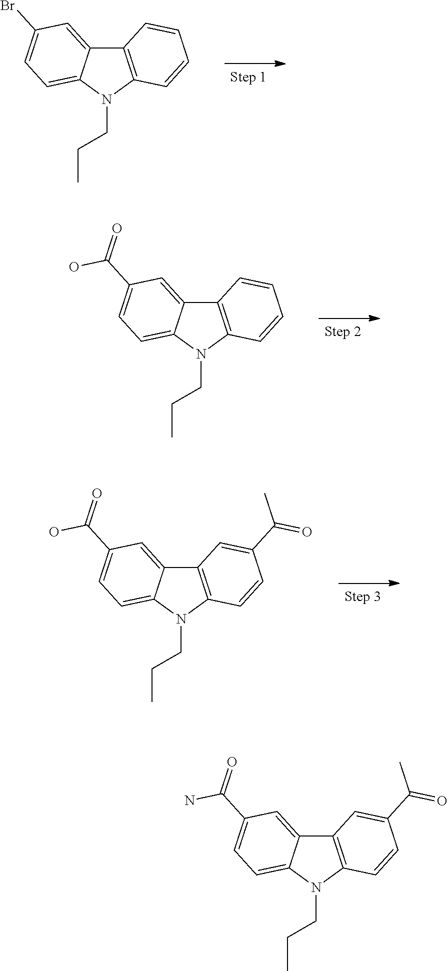 [SMILES: CCCn1c2ccccc2c2cc(Br)ccc21 ; CCCn1c2ccccc2c2cc(C(=O)O)ccc21 ; CCCn1c2ccc(C(C)=O)cc2c2cc(C(=O)O)ccc21 ; CCCn1c2ccc(C(C)=O)cc2c2cc(C(N)=O)ccc21]

Step 1. Compound 23a-1. 9-Propyl-9H-carbazole-3-carboxylic acid. To a solution of n-butyllithium (1.6 M in hexanes, 9.13 mL, 14.6 mmol) under argon at −78° C. was added a solution of 9-propyl-3-bromocarbazole (Compound 1-22, 3.5 g, 12.1 mmol) in dry tetrahydrofuran (80 mL), and the mixture stirred at −78° C. for 30 min. Carbon dioxide was bubbled through the solution for 15 min. The reaction mixture was allowed to warm to room temperature and evaporated. The residue was partitioned between ethyl acetate (50 mL) and water (25 mL). The layers were separated and the aqueous layer was extracted with ethyl acetate (2×40 mL). The combined organic layers were dried over sodium sulfate and evaporated to give the title compound (2.16 g, 8.54 mmol, 70%) as an off-white powder. LCMS: 100%, ESMS m/z 254 (M+H)⁺.

Step 2. Compound 23b-1. 6-Acetyl-9-propyl-9H-carbazole-3-carboxylic acid. To a solution of 9-propyl-9H-carbazole-3-carboxylic acid (Compound 23a-1, 200 mg, 0.79 mmol) in dichloroethane (6 mL) at 0° C. was added aluminum chloride (420 mg, 3.16 mmol) and the mixture stirred at 0° C. for 10 min. To the reaction mixture was added a solution of acetyl chloride (170 μL, 2.37 mmol) in dichloroethane (1.5 mL). The mixture stirred at room temperature for 1 h. The reaction mixture was poured into crushed ice (15 mL) and extracted with dichloromethane (3×10 mL). The combined organic layers were dried over sodium sulfate and evaporated. The crude product was recrystallized from ethyl acetate (2 mL) to give the title compound (198 mg, 0.67 mmol, 85%) as a white powder. LCMS: 97%, Rt 1.529, ESMS m/z 296 (M+H)⁺; ¹H NMR (500 MHz, DMSO-d₆) 6 ppm 12.71 (br. s, 1H), 9.04 (s, 1H), 8.96 (s, 1H), 8.04-8.15 (m, 2H), 7.71-7.81 (m, 2H), 4.46 (t, J=7.1 Hz, 2H), 2.70 (s, 3H), 1.82 (sext, J=7.3 Hz, 2H), 0.87 (t, J=7.3 Hz, 3H).

Step 3. Compound 23-1. 6-Acetyl-9-propyl-9H-carbazole-3-carboxylic acid amide. A mixture of 6-acetyl-9-propyl-9H-carbazole-3-carboxylic acid (Compound 23b-1, 90 mg, 0.31 mmol), O-(7-azabenzotriazol-1-yl)-N,N,N′,N′-tetramethyluronium hexafluorophosphate (HATU, 240 mg, 0.63 mmol) and N-methylmorpholine (104 μL, 0.95 mmol) in acetonitrile (3.2 mL) was stirred at room temperature for 1 h. To the reaction mixture was added a solution of ammonia in 1,4-dioxane (3 M, 4 mL) and the mixture stirred at room temperature for 16 h. The mixture was evaporated, diluted with ethyl acetate (15 mL) and washed with water (7 mL), 5% sodium hydroxide solution (7 mL) and brine (7 mL). The organic layer was dried over sodium sulfate, evaporated and crystallized from ethyl acetate (1 mL) to give the title compound (55 mg, 0.187 mmol, 60%) as an off-white powder. LCMS 99%, Rt 1.417, ESMS m/z 295 (M+H)⁺; ¹H NMR (500 MHz, DMSO-d₆) δ ppm 8.90 (d, J=1.5 Hz, 1H), 8.89 (d, J=1.5 Hz, 1H), 8.11 (dd, J=8.6, 1.7 Hz, 1H), 8.07 (dd, J=8.6, 1.7 Hz, 1H), 7.97 (br. s, 1H), 7.76 (d, J=8.6 Hz, 1H), 7.73 (d, J=8.6 Hz, 1H), 7.27 (br. s, 1H), 4.45 (t, J=7.1 Hz, 2H), 2.69 (s, 3H), 1.82 (sext, J=7.3 Hz, 2H), 0.87 (t, J=7.3 Hz, 3H).

### Example 24

### Compound 24-1. 5-Propyl-5H-pyrido[3,2-b]indole-8-carbonitrile

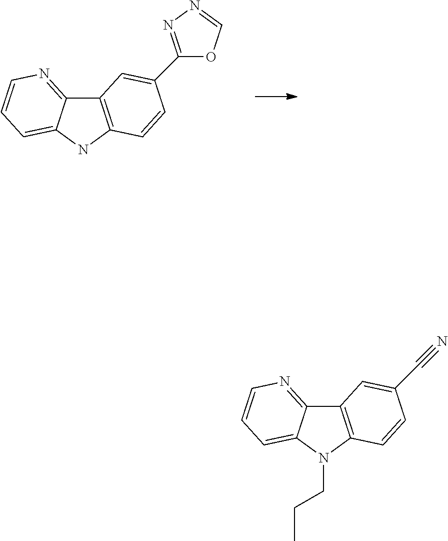 [SMILES: c1cnc2c(c1)[nH]c1ccc(-c3nnco3)cc12 ; CCCn1c2ccc(C#N)cc2c2ncccc21]

A mixture of 8-[1,3,4]oxadiazol-2-yl-5H-pyrido[3,2-b]indole (Compound 10c-1, 50 mg, 0.21 mmol) and sodium hydride (60% dispersion, 18 mg, 0.42 mmol) in N,N-dimethylformamide (1 mL) was stirred at room temperature for 20 min. 1-Bromopropane (39 mg, 29 μL, 0.32 mmol) was added dropwise and the mixture was stirred for 13 h and evaporated. The crude product was purified by column chromatography eluting with hexane:ethyl acetate (4:1) to give the title compound (30 mg, 0.13 mmol, 60%) as a white powder. LCMS: 100%, Rt 1.434, ESMS m/z 236 (M+H)⁺; ¹H NMR (500 MHz, CDCl₃) δ ppm 8.72 (s, 1H), 8.65 (d, J=4.4 Hz, 1H), 7.78 (t, J=7.6 Hz, 2H), 7.52 (d, J=8.8 Hz, 1H), 7.45 (dd, J=8.3, 4.9 Hz, 1H), 4.33 (t, J=7.1 Hz, 2H), 1.94 (sext, J=7.2 Hz, 2H), 0.99 (t, J=7.4 Hz, 3H).

### Example 25

### Compound 25-1. 6-Acetyl-9-propyl-9H-carbazole-4-carbonitrile

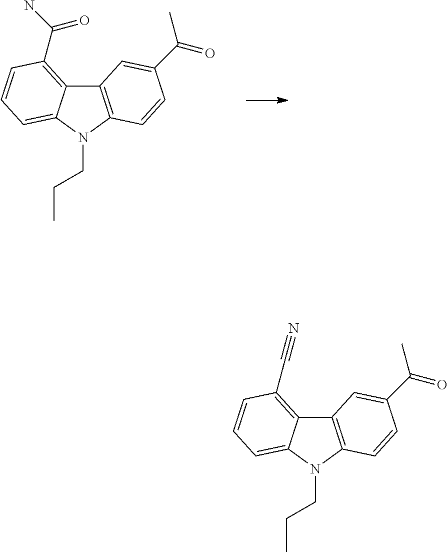 [SMILES: CCCn1c2ccc(C(C)=O)cc2c2c(C(N)=O)cccc21 ; CCCn1c2ccc(C(C)=O)cc2c2c(C#N)cccc21]

To a solution of 6-acetyl-9-propyl-9H-carbazole-4-carboxamide (Compound 19-1, 45 mg, 0.153 mmol) in dichloromethane (1.5 mL) was added triethylamine (136 μL, 0.98 mmol) and trifluoroacetic anhydride (69 μL, 0.48 mmol) and the mixture stirred at room temperature for 1 h. The mixture was evaporated and the residue was purified by column chromatography eluting with heptane:ethyl acetate (5:1). The product was triturated with hexane (0.5 mL) to give the title compound (11 mg, 0.040 mmol, 26%) as a white powder. LCMS: 100%, Rt 1.823, ESMS m/z 277 (M+H)⁺; ¹H NMR (500 MHz, CDCl₃) δ ppm 9.23 (d, J=1.0 Hz, 1H), 8.29 (dd, J=8.8, 1.5 Hz, 1H), 7.70 (d, J=8.3 Hz, 1H), 7.62 (dd, J=7.4, 1.0 Hz, 1H), 7.57 (t, J=7.4 Hz, 1H), 7.52 (d, J=8.8 Hz, 1H), 4.36 (t, J=7.3 Hz, 2H), 2.78 (s, 3H), 1.95 (sext, J=7.4 Hz, 2H), 0.99 (t, J=7.3 Hz, 3H).

Compounds 25-2-25-3 listed in the table below were prepared in a similar manner.

| | | | | | Anal. | |
|---|---|---|---|---|---|---|
| | | | | | Meth- | |
| Ex. | Structure | MW | Ion | Rt | od | Yield |
| 25-2 | 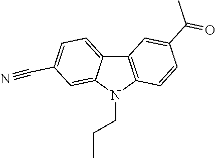 [SMILES: CCCn1c2ccc(C(C)=O)cc2c2ccc(C#N)cc21] | 276 | 277 | 1.726 | A | 45 |
| 25-3 | 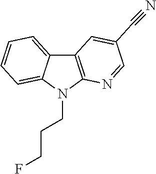 [SMILES: N#Cc1cnc2c(c1)c1ccccc1n2CCCF] | 253 | 254 | 1.676 | A | 19 |

### Example 26

### Compound 26-1. 9-(3-Fluoropropyl)-9H-carbazole-3-carbonitrile

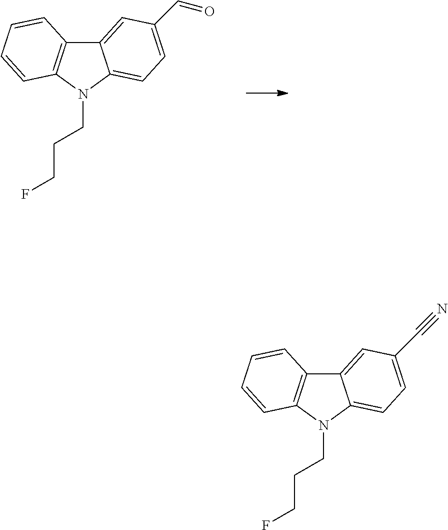 [SMILES: O=Cc1ccc2c(c1)c1ccccc1n2CCCF ; N#Cc1ccc2c(c1)c1ccccc1n2CCCF]

A mixture of 9-(3-fluoropropyl)-9H-carbazole-3-carbaldehyde (Compound 11a-1, 85 mg, 0.33 mmol), hydroxylamine hydrochloride (27.8 mg, 0.40 mmol), acetic acid (66 μL, 1.16 mmol) and pyridine (48 μL, 0.60 mmol) in N,N-dimethylformamide (2.5 mL) was stirred at 140° C. for 16 h. The reaction mixture was evaporated and the residue taken up in dichloromethane (10 mL). The organic layer was washed with water (5 mL) and 0.5 N hydrochloric acid (5 mL), dried over sodium sulfate and evaporated. The residue was purified by column chromatography eluting with heptane: ethyl acetate (12.5:1). The product was triturated with hexane (1 mL) to give the title compound (40 mg, 0.159 mmol, 54%) as a white powder. LCMS: 100%, Rt 1.793, ESMS m/z 253 (M+H)⁺; ¹H NMR (500 MHz, CDCl₃) 6 ppm 8.41 (d, J=1.0 Hz, 1H), 8.13 (d, J=7.8 Hz, 1H), 7.73 (dd, J=8.3, 1.5 Hz, 1H), 7.58 (t, J=7.6 Hz, 1H), 7.47-7.53 (m, 2H), 7.36 (t, J=7.6 Hz, 1H), 4.53 (t, J=6.6 Hz, 2H), 4.42 (dt, J=47.2, 5.9 Hz, 2H), 2.27 (dquint, J=27.8, 5.9 Hz, 2H).

### Example 27

### Compound 27-1. 1-(5-Hydroxy-9-propyl-9H-carbazol-3-yl)-ethanone

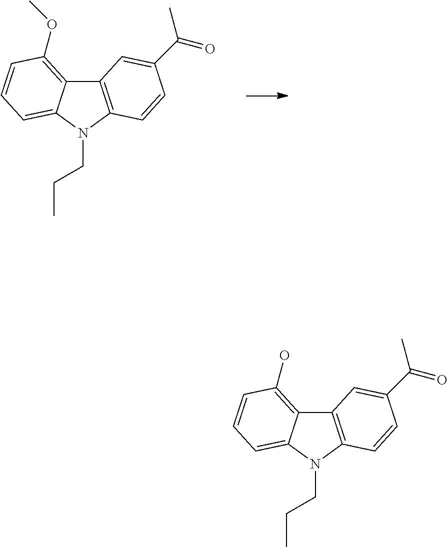 [SMILES: CCCn1c2ccc(C(C)=O)cc2c2c(OC)cccc21 ; CCCn1c2ccc(C(C)=O)cc2c2c(O)cccc21]

A solution of 1-(5-methoxy-9-propyl-9H-carbazol-3-yl)-ethanone (Compound 21-1, 40 mg, 0.142 mmol) in dichloromethane (2 mL) was cooled to −78° C. and a solution of boron tribromide (27 μL, 0.284 mmol) in dichloromethane (1 mL) was added. The mixture was stirred at −78° C. for 2 h. Boron tribromide (27 μL, 0.284 mmol) was added as a solution in dichloromethane (1 mL) and the mixture was stirred at −78° C. for 3 h. The mixture was warmed to 0° C. and stirred for 14 h. The reaction was quenched with 2 N hydrochloric acid (6 mL) and the mixture extracted with dichloromethane (2×10 mL). The combined organic layers were dried over sodium sulfate and evaporated. The residue was purified by column chromatography eluting with hexane:ethyl acetate (4:1). The solid was recrystallized from acetonitrile (0.25 mL) to give the title compound (2.4 mg, 0.009 mmol, 6%) as an off-white solid. LCMS: 99%, Rt 1.624, ESMS m/z 268 (M+H)⁺; ¹H NMR (500 MHz, CD₃OD) δ ppm 8.95 (d, J=2.0 Hz, 1H), 8.08 (dd, J=8.8, 1.7 Hz, 1H), 7.50 (d, J=8.8 Hz, 1H), 7.30 (t, J=8.1 Hz, 1H), 7.02 (d, J=8.1 Hz, 1H), 6.67 (d, J=8.1 Hz, 1H), 4.33 (t, J=7.1 Hz, 2H), 2.70 (s, 3H), 1.90 (sext, J=7.3 Hz, 2H), 0.95 (t, J=7.4 Hz, 3H).

Compounds 27-2-27-3 listed in the table below were prepared in a similar manner.

| | | | | | Anal. | |
|---|---|---|---|---|---|---|
| Ex. | Structure | MW | Ion | Rt | Method | Yield |
| 27-2 | 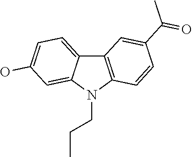 [SMILES: CCCn1c2ccc(C(C)=O)cc2c2ccc(O)cc21] | 267 | 268 | 1.499 | A | 7 |
| 27-3 | 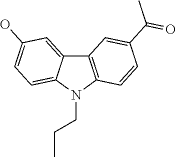 [SMILES: CCCn1c2ccc(O)cc2c2cc(C(C)=O)ccc21] | 267 | 268 | 1.559 | A | 28 |

### Example 28

### Compound 28-1. 6-Acetyl-9-propyl-9H-carbazol-3-yl acetate

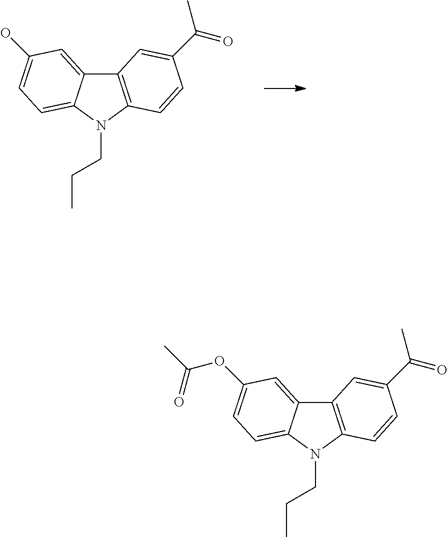 [SMILES: CCCn1c2ccc(O)cc2c2cc(C(C)=O)ccc21 ; CCCn1c2ccc(OC(C)=O)cc2c2cc(C(C)=O)ccc21]

To a solution of 1-(6-hydroxy-9-propyl-9H-carbazol-3-yl)ethanone (Compound 27-3, 40 mg, 0.150 mmol) in dichloromethane (1.5 mL) was added acetic anhydride (42 μL, 0.448 mmol) and pyridine (15 μL, 0.180 mmol) and the mixture stirred at room temperature for 1 h. The mixture was diluted with chloroform (5 mL) and washed with water (5 mL). The organic layer was dried over sodium sulfate and evaporated. The residue was purified by column chromatography eluting with hexane:ethyl acetate (7:1). The product was triturated with diethyl ether (0.5 mL) to give the title compound (22.0 mg, 0.071 mmol, 48%) as a white powder. LCMS: 100%, Rt 1.798, ESMS m/z 310 (M+H)⁺; ¹H NMR (500 MHz, CDCl₃) δ ppm 8.69 (d, J=1.5 Hz, 1H), 8.15 (dd, J=8.8, 2.0 Hz, 1H), 7.88 (d, J=2.4 Hz, 1H), 7.42 (d, J=8.8 Hz, 2H), 7.24 (dd, J=8.8, 2.0 Hz, 1H), 4.30 (t, J=7.1 Hz, 2H), 2.72 (s, 3H), 2.38 (s, 3H), 1.93 (sext, J=7.3 Hz, 2H), 0.99 (t, J=7.4 Hz, 3H).

### Example 29

### Compound 29-1, 1-(6-Amino-9-propyl-9H-carbazol-3-yl)-ethanone and Compound 29-2, N-(6-Acetyl-9-propyl-9H-carbazol-3-yl)-acetamide

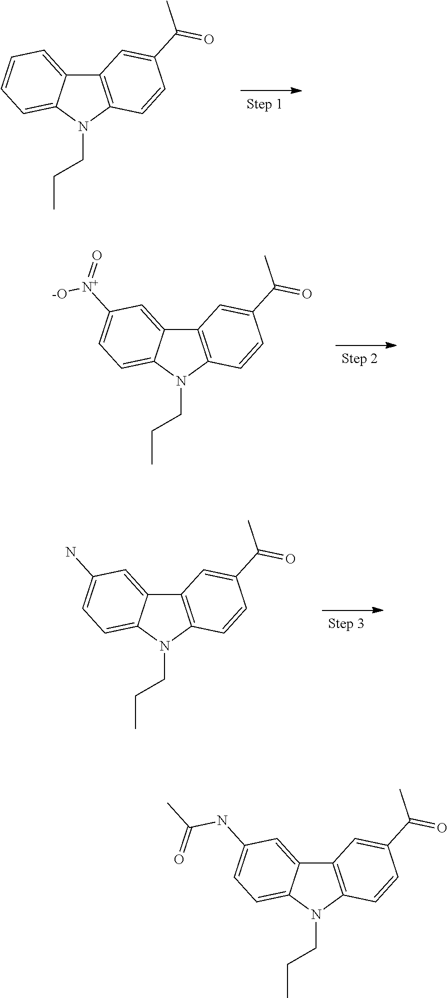

Step 1. Compound 29a-1.1-(6-Nitro-9-propyl-9H-carbazol-3-yl)-ethanone. To a solution of 1-(9-propyl-9H-carbazol-3-yl)-ethanone (Compound 1-3, 200 mg, 0.796 mmol) in acetic acid (6 mL) was added nitric acid (65%, 720 μL) and the mixture stirred at room temperature for 2 h. The reaction mixture was poured into ice water (15 mL) and the precipitate collected to give the crude product (204 mg, 0.69 mmol, 79%) as a yellow powder, which was used in the next step without further purification. LCMS: 83%, Rt 1.796, ESMS m/z 297 (M+H)⁺. A 30 mg portion (30 mg, 0.101 mmol) was purified by column chromatography eluting with hexane:dichloromethane (1:1). The product was triturated with hexane (0.5 mL) to give the title compound as a yellow powder. ¹H NMR (500 MHz, CDCl₃) δ ppm 9.09 (d, J=2.2 Hz, 1H), 8.80 (s, 1H), 8.44 (dd, J=9.0, 2.2 Hz, 1H), 8.24 (d, J=8.8 Hz, 1H), 7.52 (d, J=8.8 Hz, 1H), 7.49 (d, J=8.8 Hz, 1H), 4.38 (t, J=7.1 Hz, 2H), 2.76 (s, 3H), 1.98 (sext, J=7.3 Hz, 2H), 1.02 (t, J=7.4 Hz, 3H).

Step 2. Compound 29-1. 1-(6-Amino-9-propyl-9H-carbazol-3-yl)-ethanone. To a solution of 1-(6-nitro-9-propyl-9H-carbazol-3-yl)ethanone (Compound 29a-1, 150 mg, 0.51 mmol) in ethyl acetate (8 mL) was added palladium on carbon (10%, 20 mg) and the mixture was stirred at room temperature for 16 h under an atmosphere of hydrogen. The mixture was filtered and evaporated. The residue was purified by column chromatography eluting with dichloromethane:ethyl acetate (20:1) to give the title compound (75 mg, 0.28 mmol, 55%) as a yellow powder. LCMS: 100%, Rt 1.174, ESMS m/z 267 (M+H)⁺; ¹H NMR (500 MHz, CDCl₃) δ ppm 8.66 (d, J=1.5 Hz, 1H), 8.08 (dd, J=8.3, 1.5 Hz, 1H), 7.48 (d, J=2.4 Hz, 1H), 7.35 (d, J=8.8 Hz, 1H), 7.25 (d, J=8.8 Hz, 1H), 6.95 (dd, J=8.3, 2.0 Hz, 1H), 4.24 (t, J=7.1 Hz, 2H), 3.69 (br. s, 2H), 2.71 (s, 3H), 1.91 (sext, 2H), 0.97 (t, 3H).

Step 3. Compound 29-2. N-(6-Acetyl-9-propyl-9H-carbazol-3-yl)-acetamide. To a solution of 1-(6-amino-9-propyl-9H-carbazol-3-yl)-ethanone (Compound 29-1, 52 mg, 0.195 mmol) in dichloromethane (1.5 mL) was added acetic anhydride (55 μL, 0.586 mmol) and pyridine (19 μL, 0.234 mmol) and the mixture was stirred at room temperature for 1 h. The mixture was diluted with chloroform (5 mL) and washed with water (5 mL). The organic layer was dried over sodium sulfate and evaporated. The residue was triturated with diethyl ether (1 mL) and recrystallized from methanol (0.4 mL) to give the title compound (20.0 mg, 0.06 mmol, 33%) as a yellow powder. LCMS: 100%, Rt 1.462, ESMS m/z 309 (M+H)⁺; ¹H NMR (500 MHz, DMSO-d₆) δ ppm 9.98 (s, 1H), 8.73 (s, 1H), 8.55 (d, J=2.0 Hz, 1H), 8.05 (dd, J=8.8, 1.5 Hz, 1H), 7.67 (d, J=8.8 Hz, 1H), 7.62 (d, J=8.8 Hz, 1H), 7.55 (dd, J=8.8, 2.0 Hz, 1H), 4.38 (t, J=7.1 Hz, 2H), 2.68 (s, 3H), 2.09 (s, 3H), 1.80 (sext, J=7.2 Hz, 2H), 0.86 (t, J=7.3 Hz, 3H).

### Example 30

This example provides a table (below) of the compounds of the present disclosure and their activity towards four different cell lines (i.e., CWR22R, HeLa, PC3, and MDA-MB-231). The IC50 for inhibition of growth is divided into 4 categories: A<1 μM, B 1-5 μM, C 5-20 μM, D>20 μM.

| | | | | MDA- |
|---|---|---|---|---|
| Compound | CWR22R | Hela | PC3 | MB-231 |
| 1-1 | B | D | D | D |
| 1-2 | A | D | D | D |
| 1-3 | A | D | | D |
| 2-1 | A | D | D | D |
| 2-2 | D | D | D | D |
| 2-5 | A | D | D | D |
| 2-6 | B | D | | D |
| 4-1 | B | D | D | D |
| 4-2 | D | D | D | D |
| 4-4 | C | D | D | D |
| 4-5 | B | D | D | D |
| 5-1 | A | D | D | D |
| 5-2 | C | D | D | D |
| 5-8 | A | D | D | D |
| 5-10 | A | D | D | D |
| 5-11 | B | D | D | D |
| 5-13 | B | D | D | D |
| 5-14 | B | D | D | D |
| 6-1 | A | D | D | D |
| 6-4 | C | D | D | D |
| 7-1 | B | D | D | D |
| 8-1 | A | D | D | D |
| 8-2 | C | D | D | D |
| 8-3 | D | D | D | D |
| 8-4 | A | D | D | D |
| 9-1 | A | D | D | D |
| 10-1 | D | D | | D |
| 10-2 | C | D | | D |
| 11-1 | A | D | D | D |
| 11-2 | A | D | D | D |
| 12-1 | D | D | D | D |
| 13-1 | B | D | D | D |
| 13-2 | A | D | D | D |
| 14-1 | A | D | D | D |
| 15-2 | D | D | D | D |
| 17-1 | A | D | D | D |
| 17-3 | C | D | D | D |
| 18-2 | A | D | D | D |
| 19-1 | B | D | D | D |
| 19-2 | A | D | D | D |
| 20-1 | C | D | D | D |
| 20-2 | D | D | D | D |
| 21-1 | C | D | D | D |
| 22-2 | A | D | D | D |
| 23-1 | C | D | D | C |
| 25-1 | B | D | D | D |
| 25-3 | B | D | D | D |
| 26-1 | A | D | D | D |
| 27-1 | D | D | D | D |

### Example 31

This example shows various cancer cell lines and their sensitivity towards compounds of the present disclosure (S represents a cell line sensitive to growth inhibition by Compound 1-3, R represents a resistant cell line).

| | | Compound |
|---|---|---|
| | | 1-3 |
| Cell line | Description | sensitivity |
| LNCaP | castration resistant prostate cancer, AR+ | S |
| C4-2 | | S |
| CWR22R | | S |
| VCaP | castration resistant prostate cancer, Armut | R |
| PC3 | castration resistant prostate cancer, AR− | R |
| Du145 | | R |
| PPC1 | | R |
| Hela | cervix adenocarcinoma, AR− | R |
| HT1080 | fibrosarcoma AR− | R |
| MRC5 | normal fibroblasts, AR− | R |
| PANC1 | pancreatic adenocarcinoma, AR− | R |
| MiaPaca | | R |
| RCC45 | renal cell carcinoma, AR− | R |
| SKRC45 | | R |
| ACHN | | R |
| NKE | normal kidney, AR− | R |
| HepG2 | hepatocellular carcinoma, AR + | S |
| Hep3B | | S |
| normal hepatocytes | | R |
| HMEC | normal breast | R |
| MCF10A | mammary gland epithelium | R |
| AU565 | Luminal breast carcinoma, AR+ | S |
| ZR7530 | | S |
| ZR751 | | S |
| BT474 | | S |
| MDAMB415 | | S |
| MDAMB453 | | S |
| T47D | | S |
| MCF7 | | S |
| SUM185PE | | S |
| HCC1419 | | R |
| UACC893 | | R |
| CAMA1 | | R |
| HCC202 | Luminal breast carcinoma, AR− | S |
| EFM192A | | S |

### Example 32

This example provides compounds of the present disclosure and testing of the compounds against cancers.

Structures and reference numbers for compounds in this example and Example 33 are as follows:

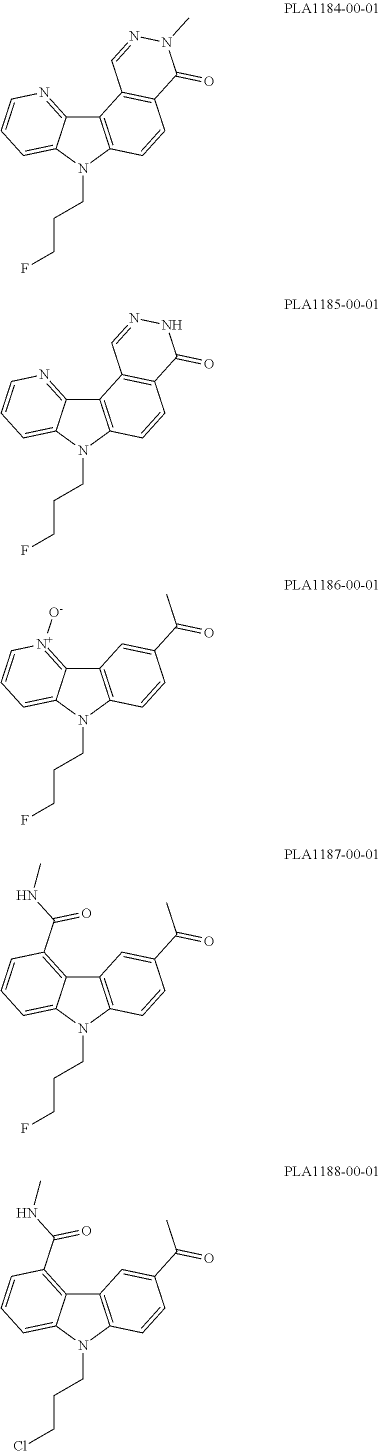

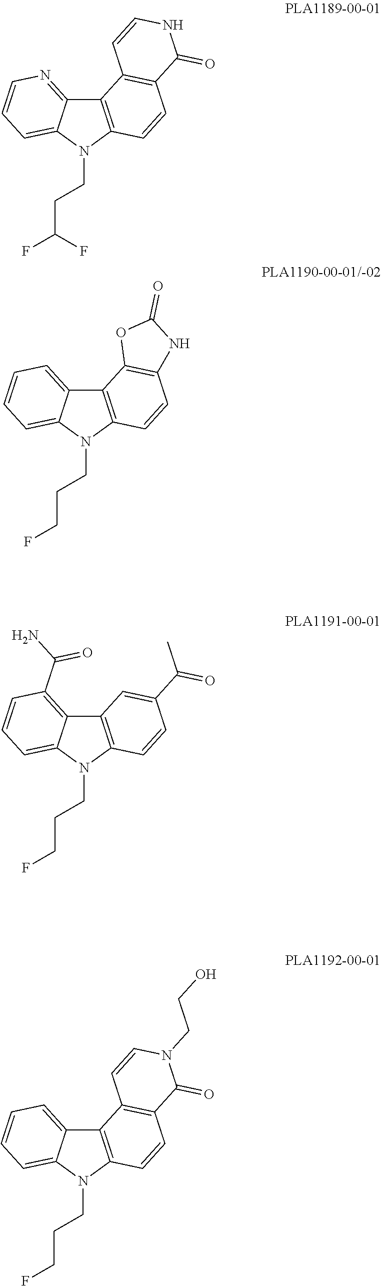 [SMILES: O=c1[nH]ccc2c1ccc1c2c2ncccc2n1CCC(F)F ; O=c1[nH]c2ccc3c(c4ccccc4n3CCCF)c2o1 ; CC(=O)c1ccc2c(c1)c1c(C(N)=O)cccc1n2CCCF ; O=c1c2ccc3c(c2ccn1CCO)c1ccccc1n3CCCF]

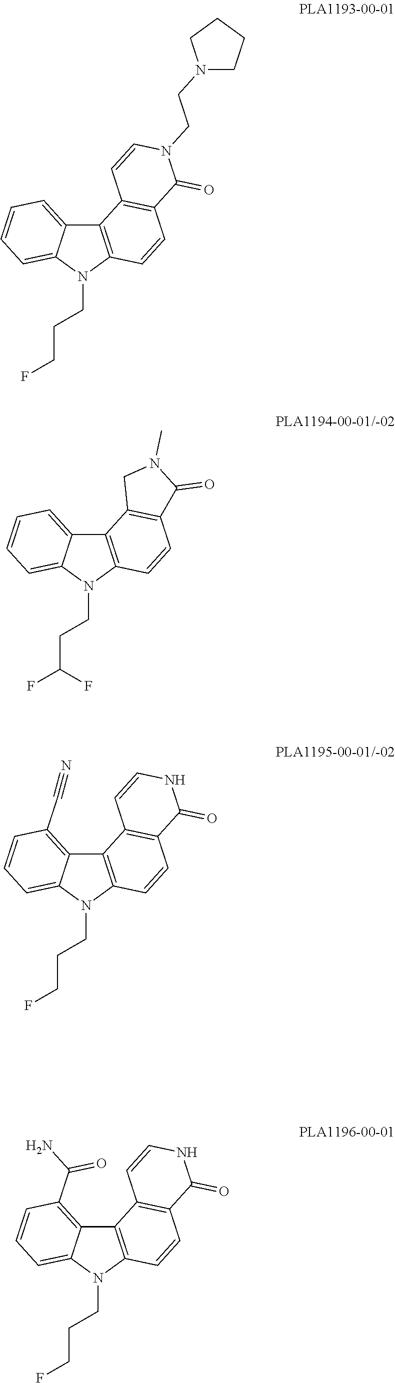 [SMILES: O=c1c2ccc3c(c2ccn1CCN1CCCC1)c1ccccc1n3CCCF ; CN1Cc2c(ccc3c2c2ccccc2n3CCC(F)F)C1=O ; N#Cc1cccc2c1c1c3cc[nH]c(=O)c3ccc1n2CCCF ; NC(=O)c1cccc2c1c1c3cc[nH]c(=O)c3ccc1n2CCCF]

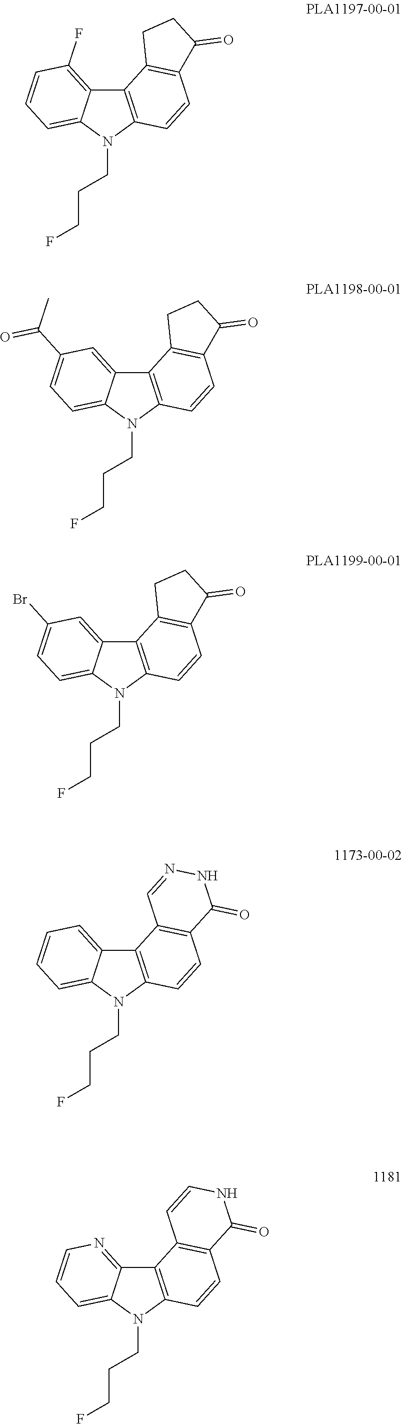 [SMILES: O=C1CCc2c1ccc1c2c2c(F)cccc2n1CCCF ; CC(=O)c1ccc2c(c1)c1c3c(ccc1n2CCCF)C(=O)CC3 ; O=C1CCc2c1ccc1c2c2cc(Br)ccc2n1CCCF ; O=c1[nH]ncc2c1ccc1c2c2ccccc2n1CCCF ; O=c1[nH]ccc2c1ccc1c2c2ncccc2n1CCCF]

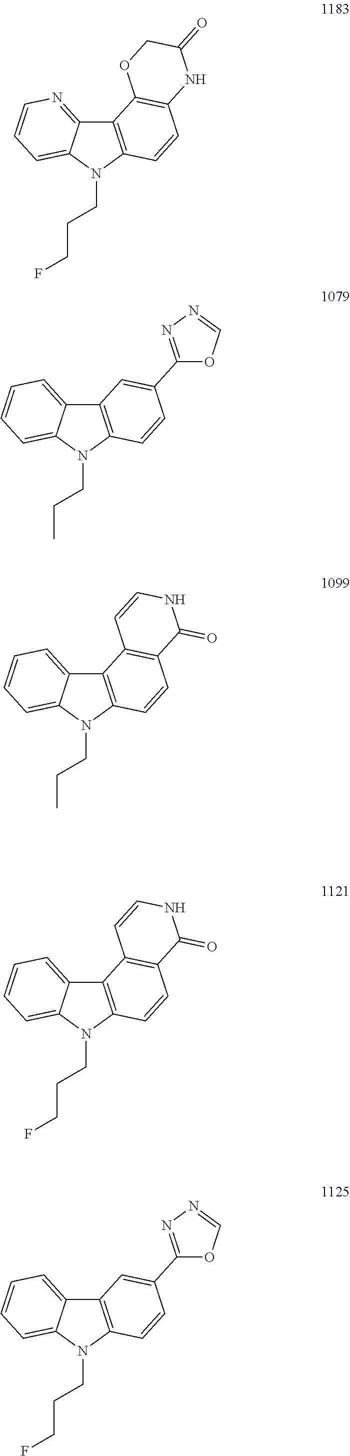 [SMILES: O=C1COc2c(ccc3c2c2ncccc2n3CCCF)N1 ; CCCn1c2ccccc2c2cc(-c3nnco3)ccc21 ; CCCn1c2ccccc2c2c3cc[nH]c(=O)c3ccc21 ; O=c1[nH]ccc2c1ccc1c2c2ccccc2n1CCCF ; FCCCn1c2ccccc2c2cc(-c3nnco3)ccc21]

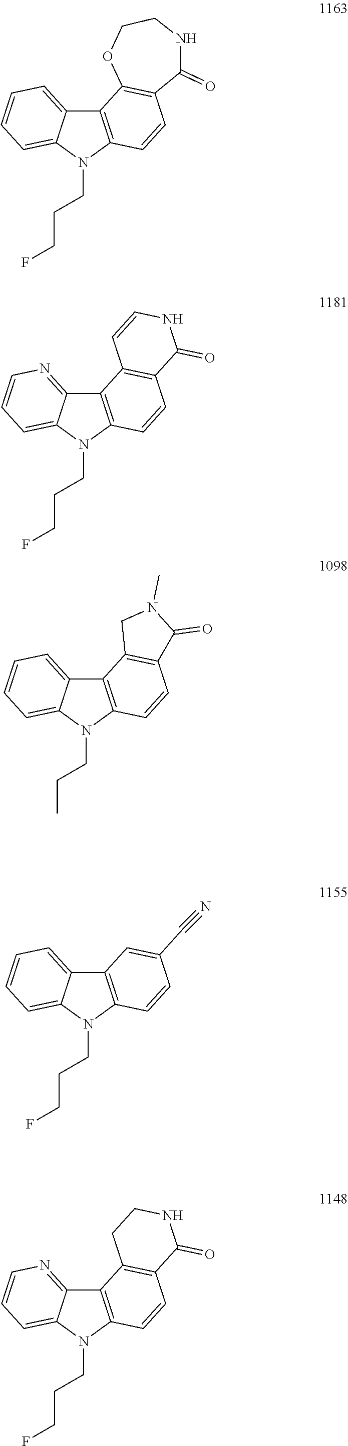 [SMILES: O=C1NCCOc2c1ccc1c2c2ccccc2n1CCCF ; O=c1[nH]ccc2c1ccc1c2c2ncccc2n1CCCF ; CCCn1c2ccccc2c2c3c(ccc21)C(=O)N(C)C3 ; N#Cc1ccc2c(c1)c1ccccc1n2CCCF ; O=C1NCCc2c1ccc1c2c2ncccc2n1CCCF]

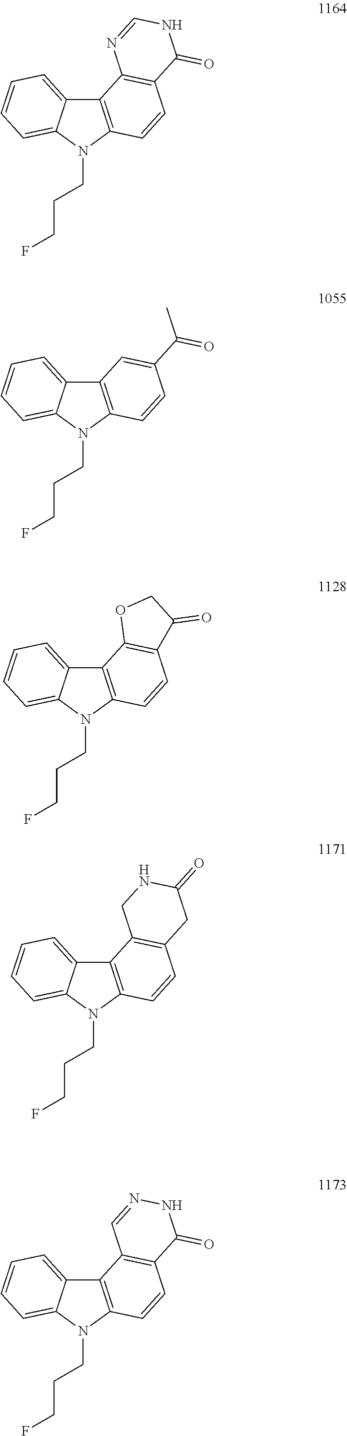 [SMILES: O=c1[nH]cnc2c1ccc1c2c2ccccc2n1CCCF ; CC(=O)c1ccc2c(c1)c1ccccc1n2CCCF ; O=C1COc2c1ccc1c2c2ccccc2n1CCCF ; O=C1Cc2ccc3c(c2CN1)c1ccccc1n3CCCF ; O=c1[nH]ncc2c1ccc1c2c2ccccc2n1CCCF]

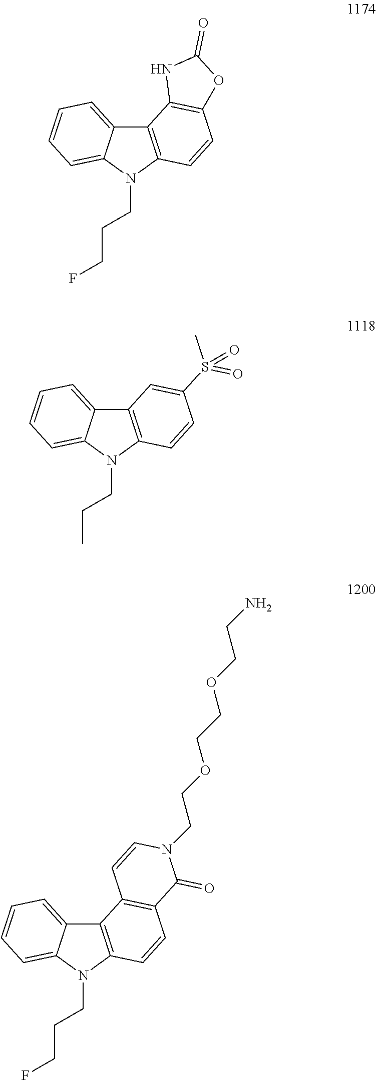 [SMILES: O=c1[nH]c2c(ccc3c2c2ccccc2n3CCCF)o1 ; CCCn1c2ccccc2c2cc(S(C)(=O)=O)ccc21 ; NCCOCCOCCn1ccc2c(ccc3c2c2ccccc2n3CCCF)c1=O]

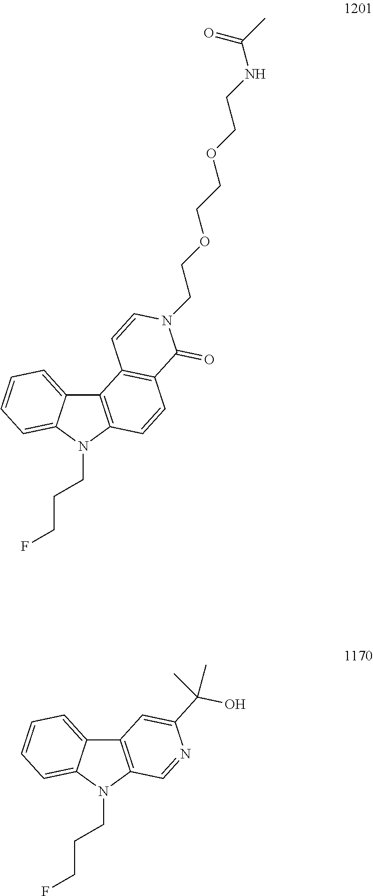 [SMILES: CC(=O)NCCOCCOCCn1ccc2c(ccc3c2c2ccccc2n3CCCF)c1=O ; CC(C)(O)c1cc2c3ccccc3n(CCCF)c2cn1]

The 4- or 5-digit reference number (where the 5-digit number has a leading 0) each refer to the same compound. Also, the reference number may have a PLA prefix or -00-01 or -00-02 suffix. For example, PLA01055, PLA1055, PLA01055-00-01, and PLA01055-00-02, PLA1055-00-01, and PLA1055-00-02 all refer to the same compound.

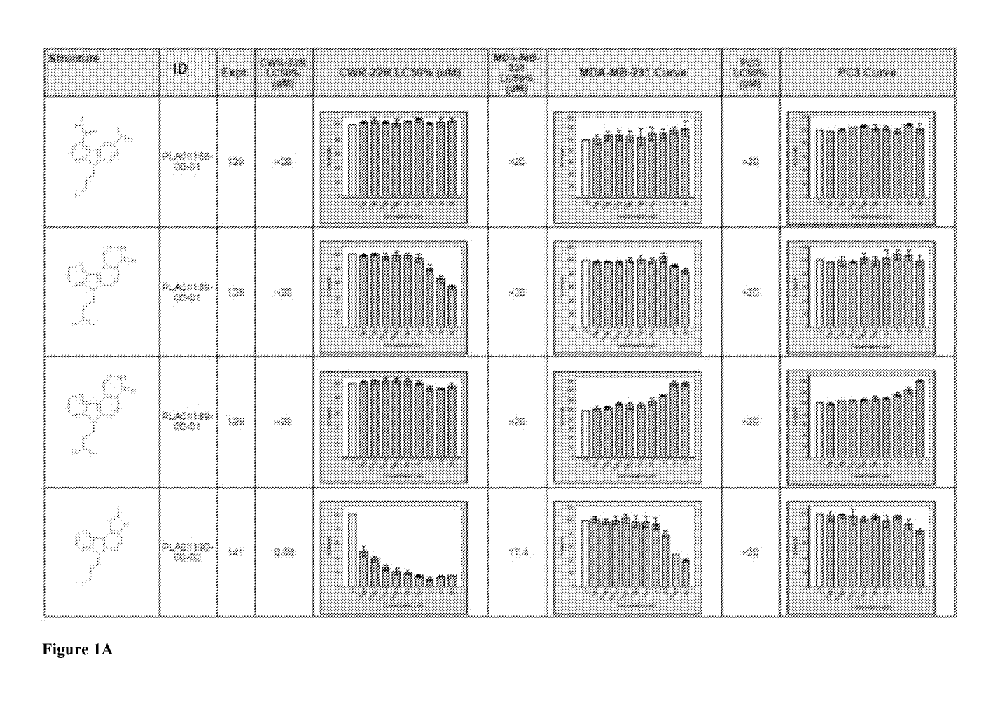

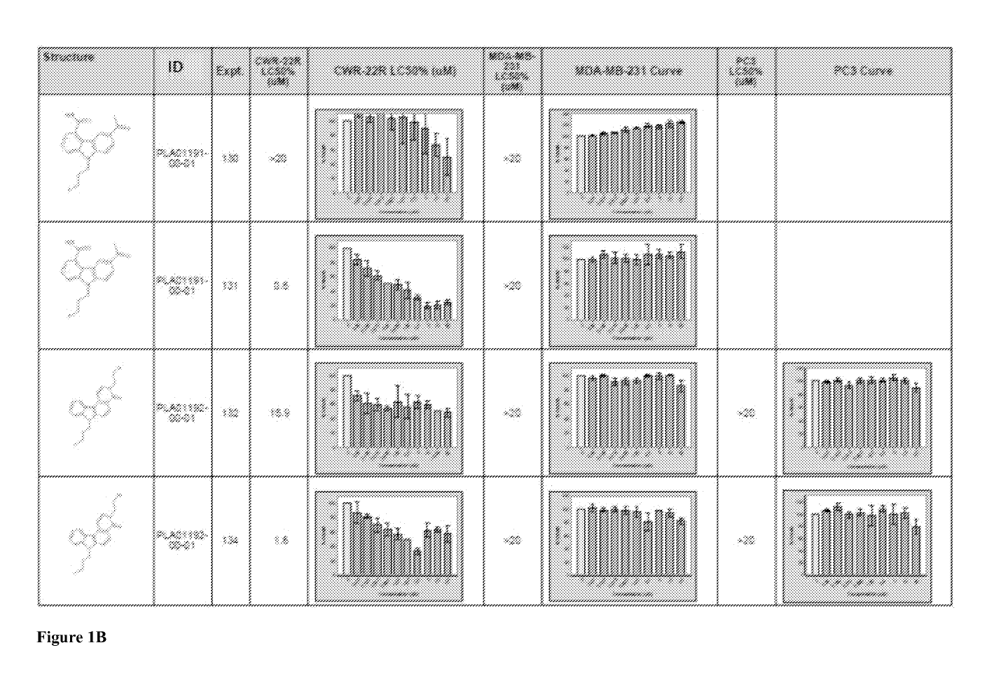

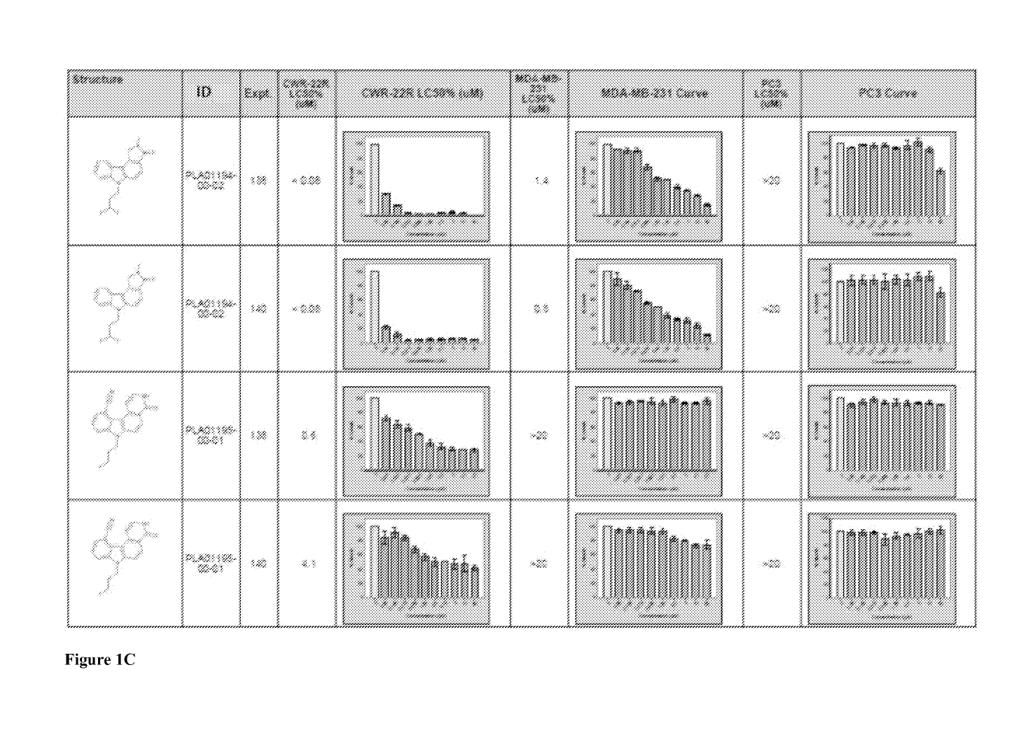

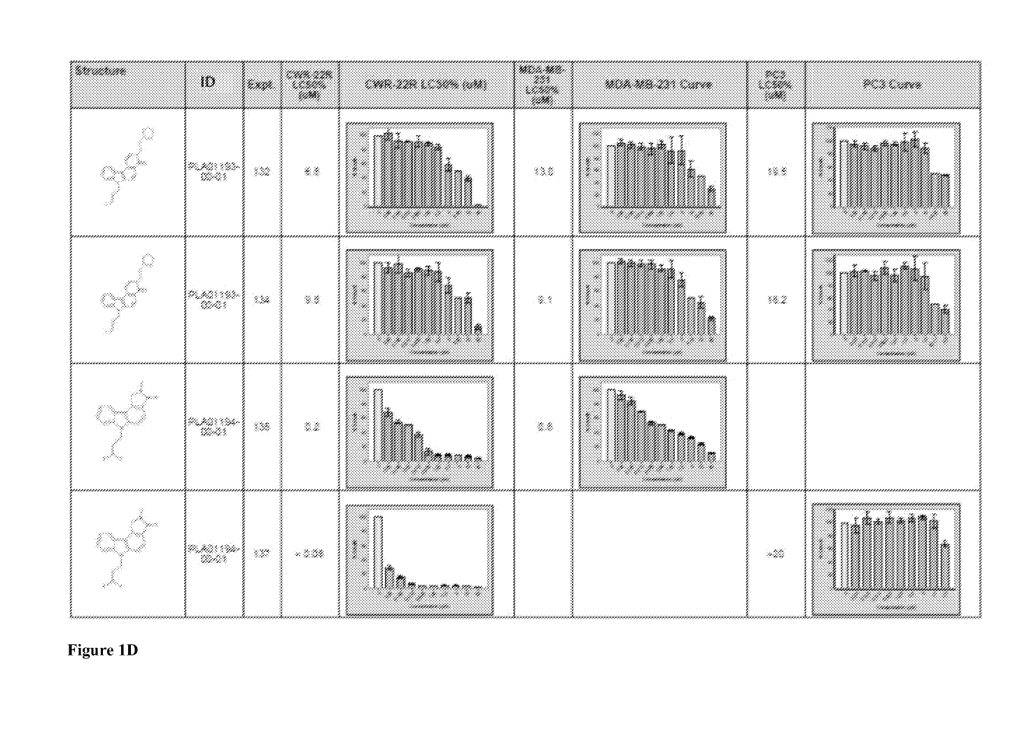

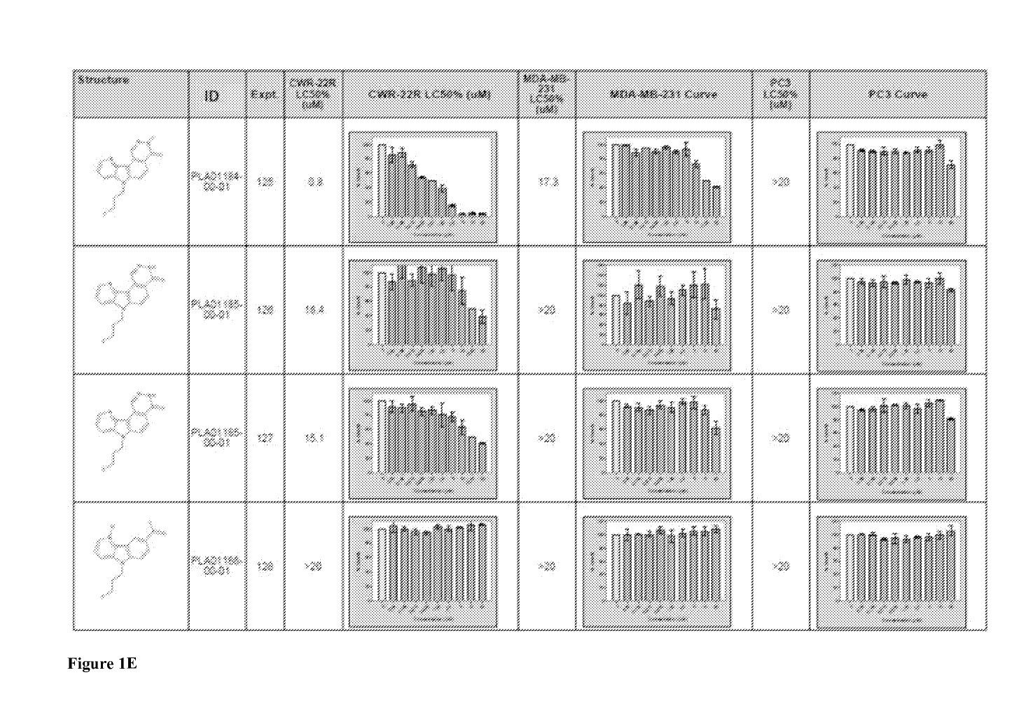

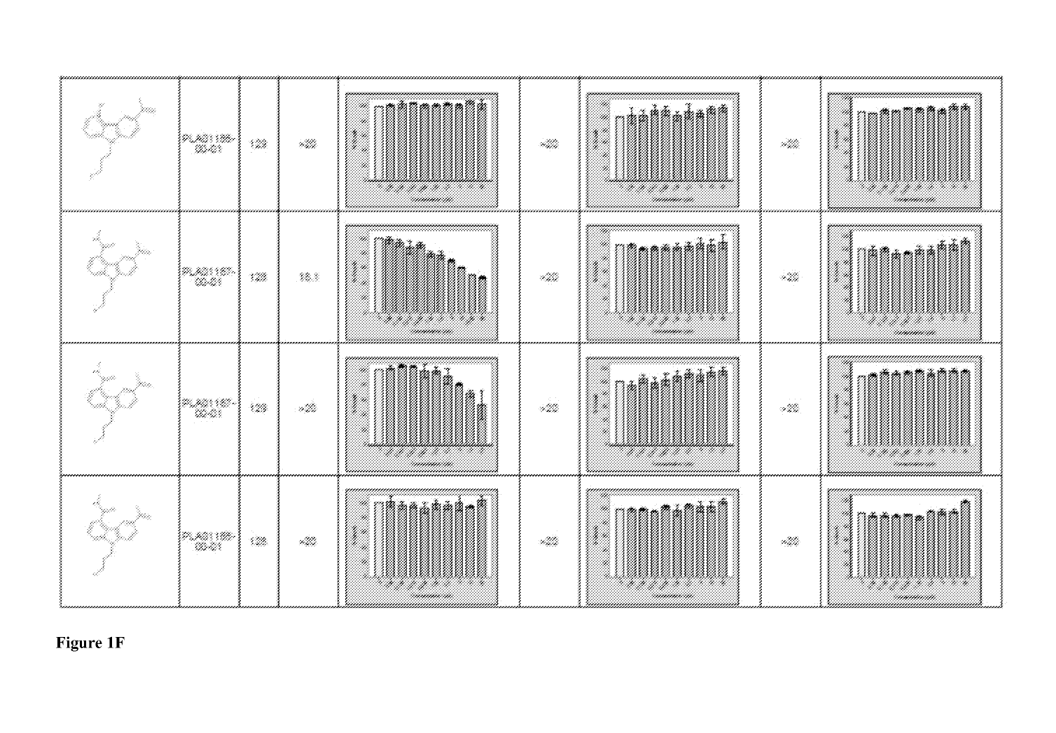

All compounds were tested for toxicity using 4 cells lines, target cells—CWR22R and non-target cells, Hela, PC3 and MDA-MB-231 (FIGS. 1A-F). Compounds with LC50 for CWR22R cells below 1 uM and no toxicity for other three cell lines at concentrations >20 uM were tested for metabolic stability and solubility (Table 1).

TABLE 1
Metabolic stability and solubility of the selected compounds.

| | Aqueous | | Aqueous | Aqueous | Aqueous |
|---|---|---|---|---|---|
| Compound: | Solubility | | Solubility | Solubility | Solubility |
| (n = 3) | (pH 7.4) | | Classification | (pH 7.4) | Classification |
| PLA1181 | <2 | μM | Low | 2.3 μM | Low |
| PLA1183 | 8.9 | μM | Low | 12.7 μM | Moderate |
| PLA1191 | 130 | μM | High | 30% | Moderate |
| PLA1192 | 15 | μM | Moderate | 27% | Moderate |
| PLA1193 | 140 | μM | High | 3% | Unstable |
| PLA1194 | 18 | μM | Moderate | 28% | Moderate |
| PLA1190 | 3.5 | μM | Low | 77% | Stable |
| PLA1195 | 2.7 | μM | Low | 63% | Moderate |
| PLA1196 | 26 | μM | Moderate | 65% | Moderate |
| PLA1197 | 4.0 | μM | Low | 73% | Stable |
| PLA1198 | 2.5 | μM | Low | 46% | Moderate |
| PLA1199 | 2.3 | μM | Low | 42% | Moderate |

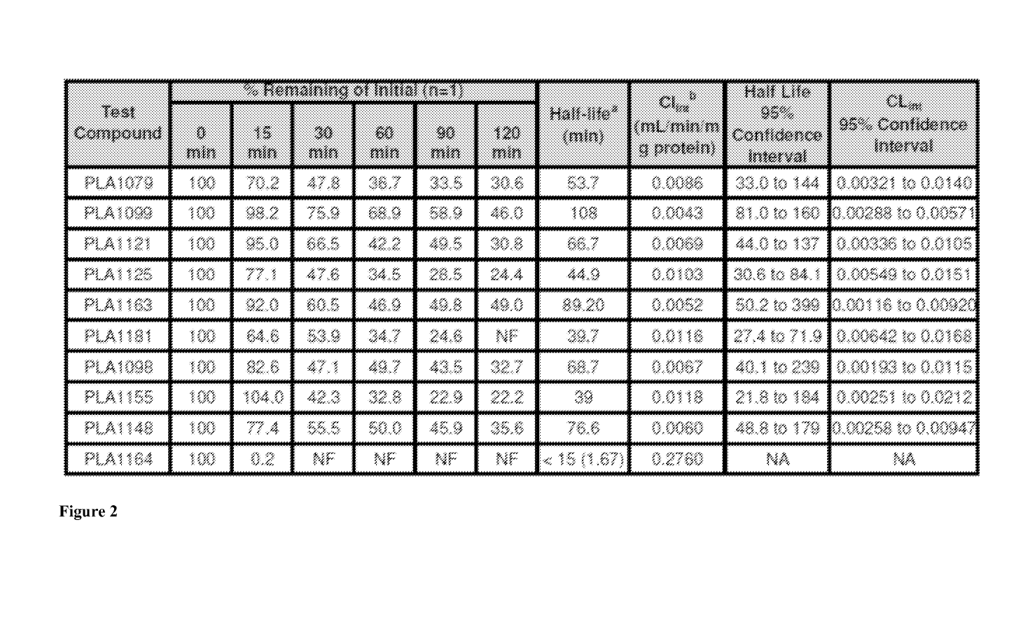

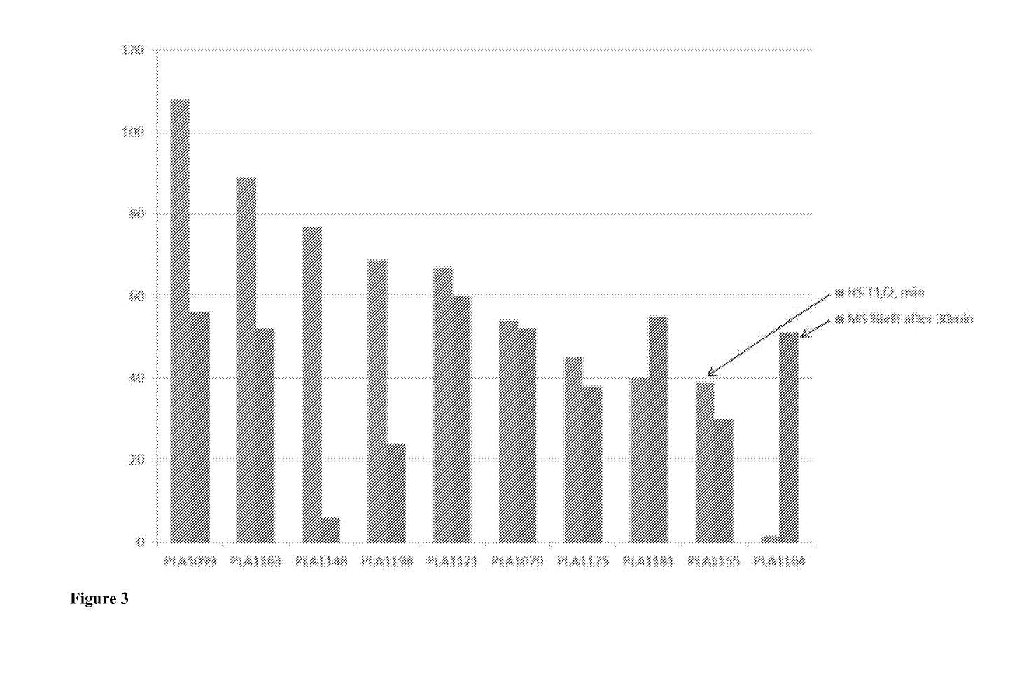

8 compounds were selected based on the solubility and metabolic stability as well as considering chemical diversity to test for time dependence stability in the presence of mouse hepatocytes (FIG. 2). Unexpectedly this assay showed week correlation with the stability of the compounds in the presence of liver microsomes (FIG. 3). Two compounds, PLAl 190 and 1197, have desirable metabolic stability, 70 and 77% left after 30 minute incubation with mouse liver microsome respectively. Formulations for in vivo administration were developed for 4 compounds, PLA1079, 1125, 1098 and 1148.

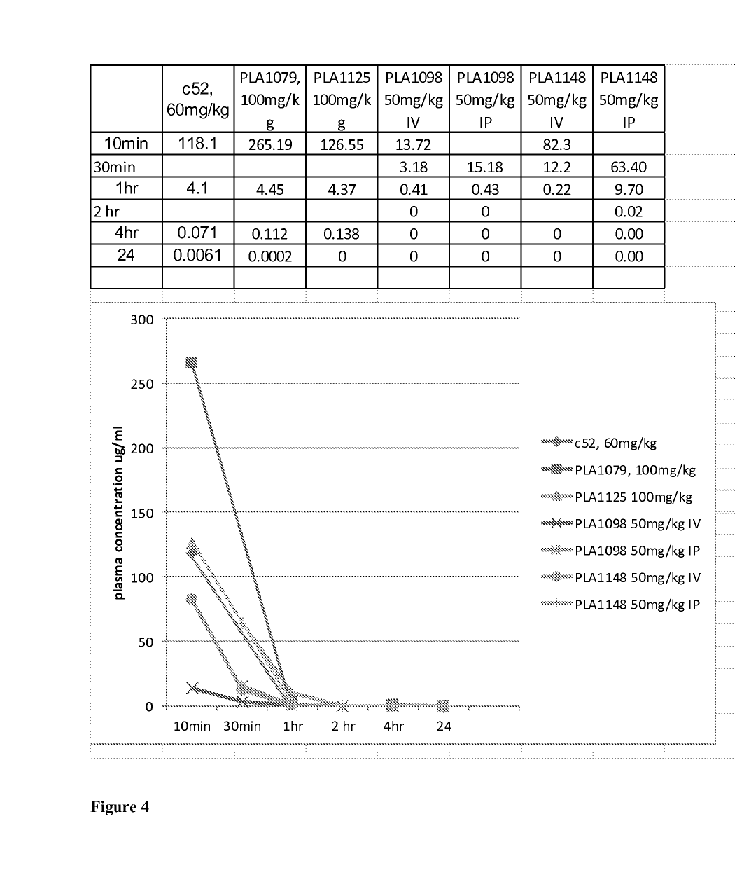

4 compounds, PLA1079, 1125, 1098 and 1148 were tested in vivo to obtain plasma concentration at different time points after intravenous and intraperitoneal injections (FIG. 4). Data were very similar for all 4 compounds, fast reduction of concentration during the first hour. Although injected doses were very high, 50-100 mg/kg they were not enough to obtain plasma concentration above LC50% in vitro at any moment, except immediately after administration (5 minutes). Positive finding was very high bioavailability of all four compounds through IP injection: plasma concentrations were similar or even higher after IP than IV administration.

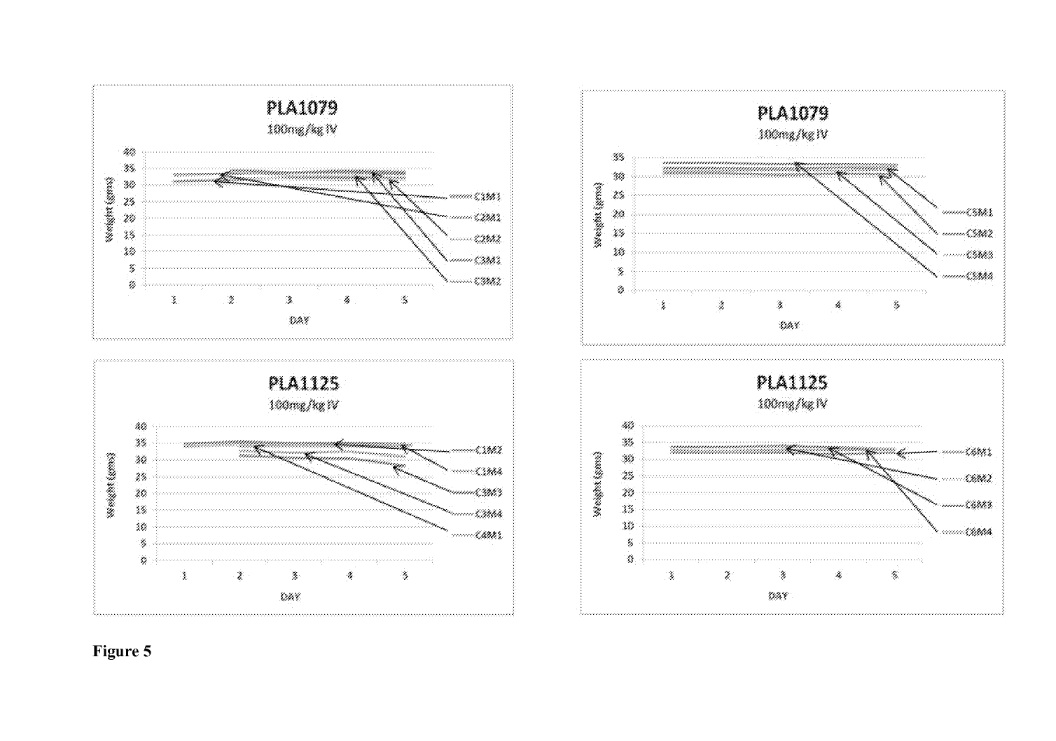

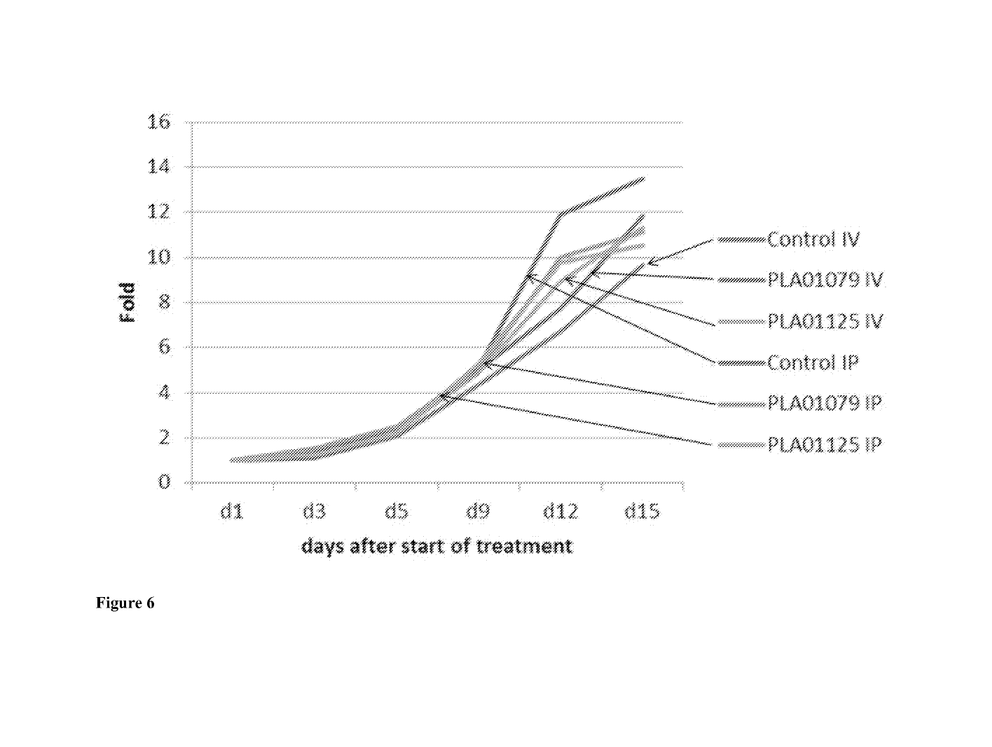

We tested these compounds in an in vivo efficacy experiment using model of CRPC in SCID mice, CWR22R. Two compounds, PLA1079 and 1125, which were synthesized in the amount of 500 mg were tested in full scale experiment with either 5 IV daily injections or 10 IP daily injections. After 5 IP or IV injections intra-tumor concentration of the compounds were measured in few mice from this experiment. It was significantly below LC50% for the same tumor cells in vitro. The absence of in vivo efficacy is attributable due to low intra-tumor concentration of the compounds (FIGS. 5, 6, and 7).

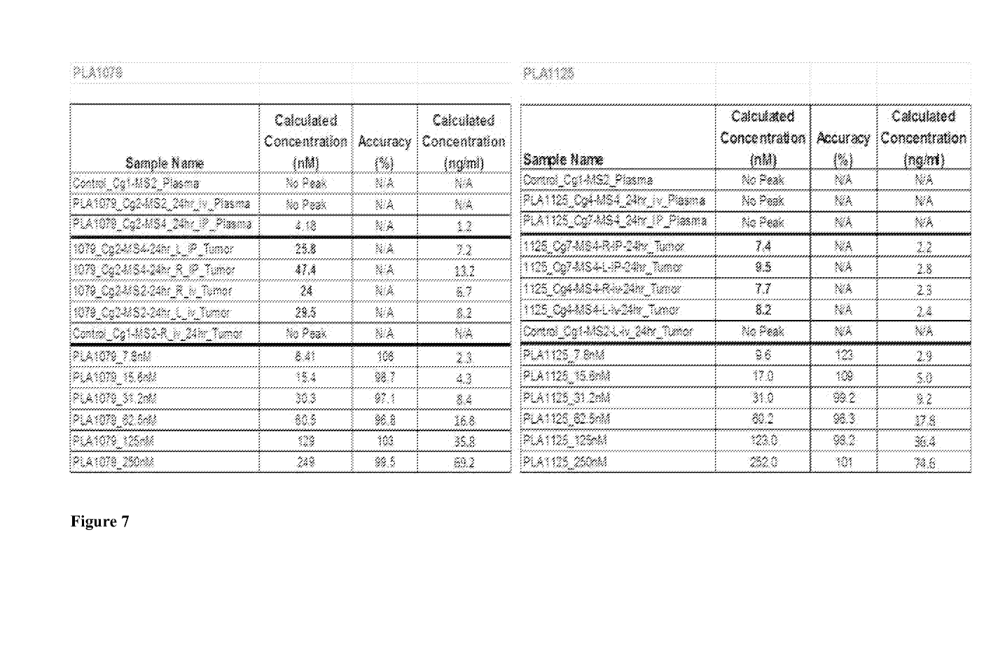

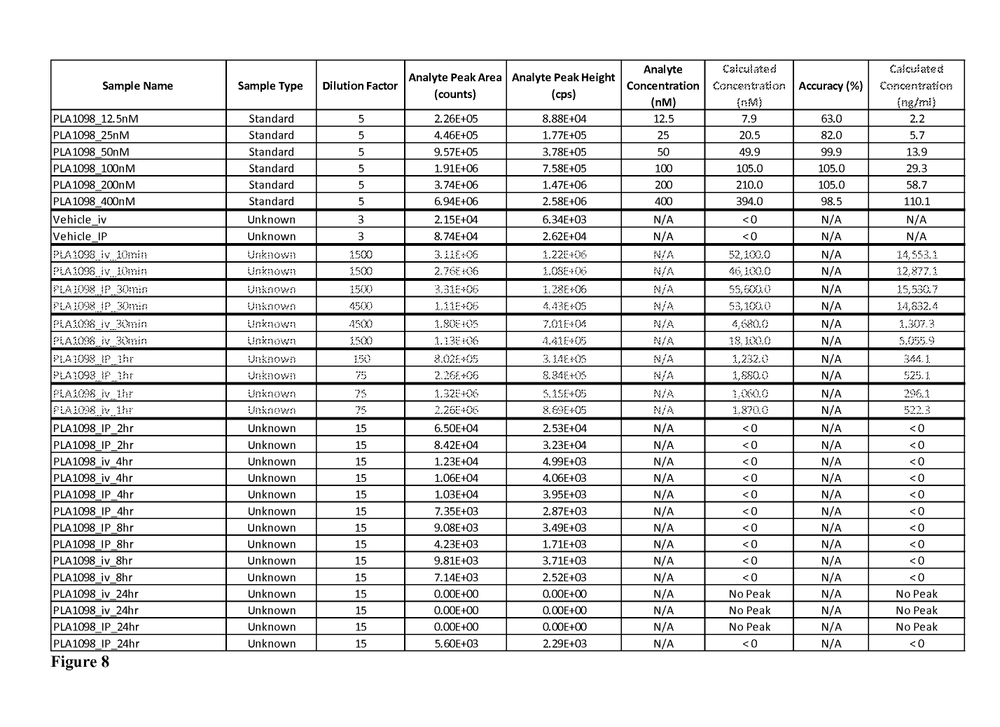

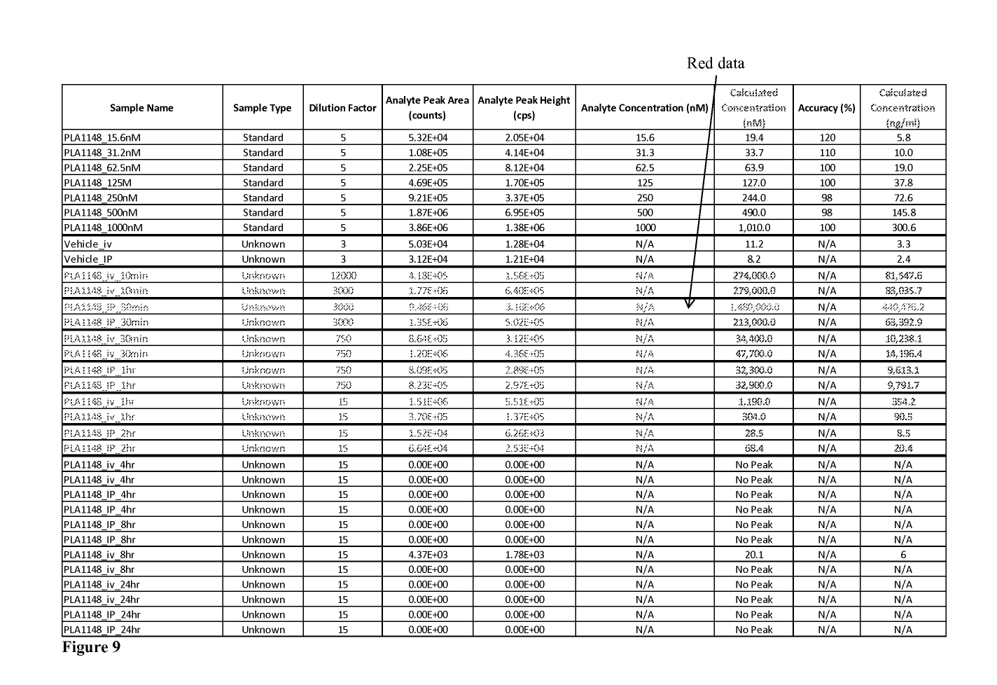

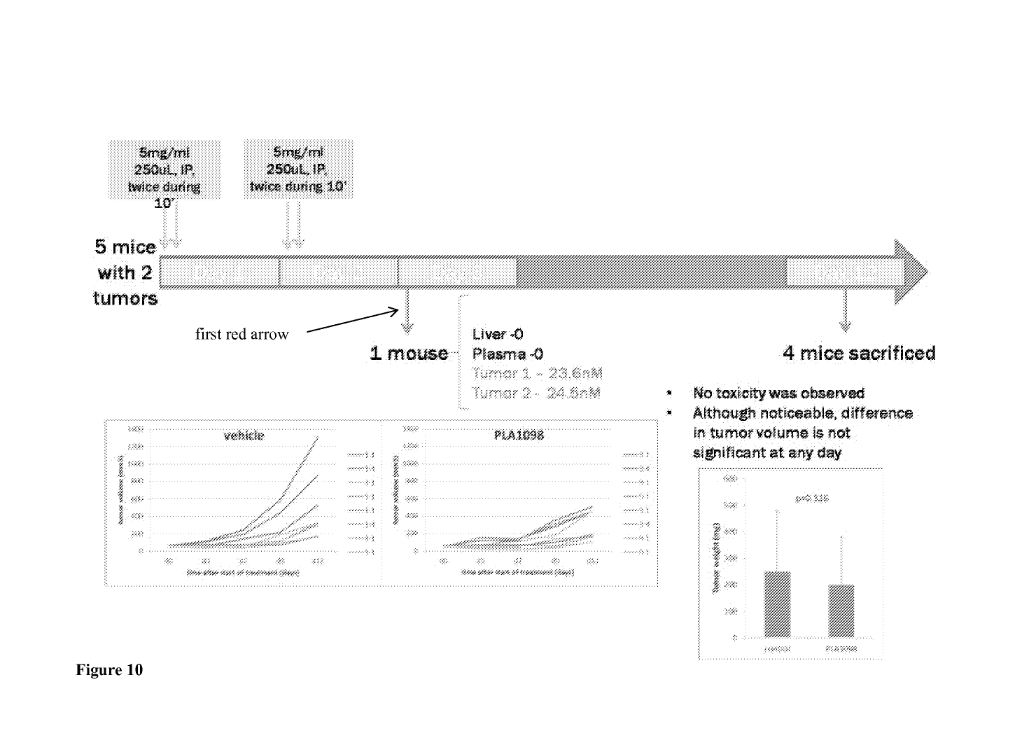

Two other compounds, PLA1148 and 1098 (PK testing in FIGS. 8 and 9) were tested in a small scale efficacy experiment. Upon PK testing of the compound PLA1148 one mouse was injected twice with 10 min interval. However the concentration of the drug in plasma of this mouse was not twice, but >4 times higher than in mice injected once. This suggests that the saturation of liver metabolizing enzymes lead to the sharp increase in the plasma concentration. It was decided to utilize this observation in an attempt to get higher plasma and tumor concentration of both compounds. Therefore in the pilot efficacy experiment in the same model of CWR22R cells mice were treated with 2 IP injections with 10 minute interval. Treatment with PLA1098 was carried out for two days. Mice were fine during the treatment, no signs of side effects were noticed and at 12 days after the treatment mice were sacrificed and tumors were excised and weighed. There were certain reduction of tumor volume and tumor weigh in the 1098 treated group vs vehicle control, but not statistically significant (FIG. 10). Short treatment period as well as small group size suggest that this difference may be significant if both conditions will be enlarged. Intratumor concentration of the compound in 2 tested tumors at 24 hours after last injection was 25 nM, what is ½ of LC50 for PLA1098 (FIG. 10). However this is so far the highest concentration found in tumor. Interestingly there were no detectable PLA1098 in plasma and liver at the same time.

Compound PLA1148 were injected only one day due to acute toxicity developed quickly after injection, symptoms suggested acute liver failure. 2 mice died next day and 3 other during next 5 days. Two mice died on the 5th day had almost undetectable tumors. There were no drug detected in one mouse at 24 hours after injections neither in tumor, nor in liver, plasma concentration was 8.5 nM. The same plasma concentration was found in the mice euthanized at 48 hours after treatment. In this mouse tumor had 84 nM of PLA1148 (LC50%-70 nM) and liver had 49 nM. This compound may be also potentially promising.

Three more compounds, PLA1099, 1121 and 1163 were poorly soluble and therefore were tested for PK using IP injection of the drug in suspension.

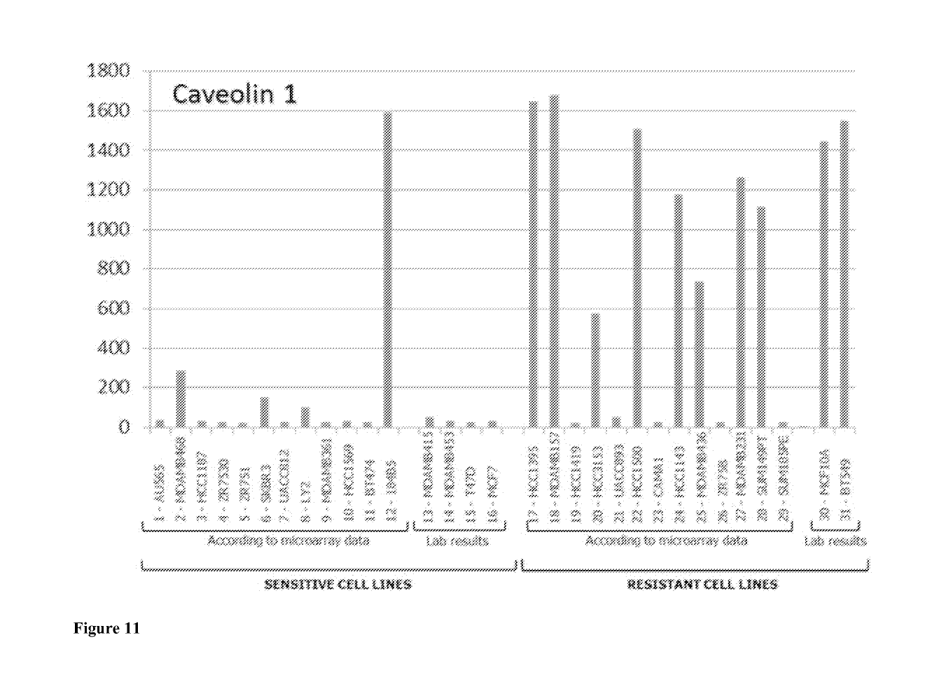

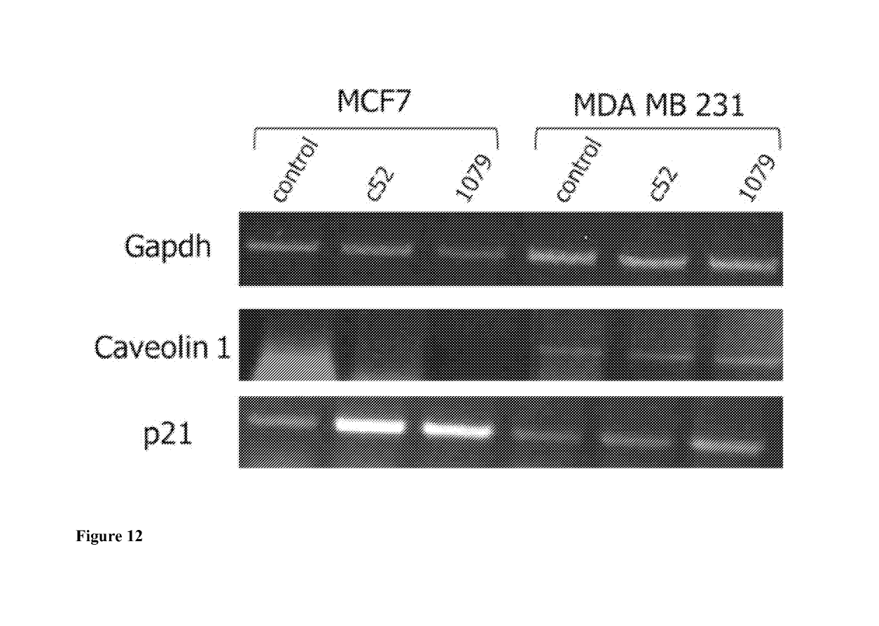

Analysis of gene expression among 50 breast cancer cell lines differed in sensitivity as well as shRNA screening of resistant breast cancer cells identified Calveolin (FIG. 11) as a potential gene associated with resistance to c52. mRNA expression of Calveolin1 was tested among our sensitive and resistant cells as well as in response to c52 treatment. As expected Calveolin1 was expressed only in resistant cells, however it was not changed upon c52 treatment (FIG. 12). Caveolinl was overexpressed in resistant cell lines, while almost all of the sensitive cell lines showed low or no expression (FIG. 12). Calveolin1 was cloned in lentiviral expression vector and are now testing if overexpression of Calveolin1 in sensitive cells will make them resistant to c52 treatment.

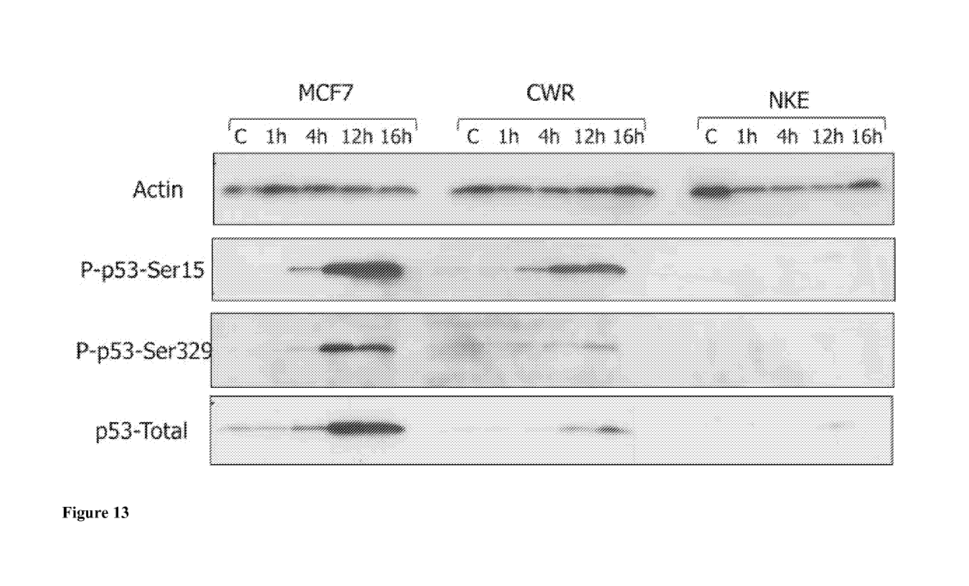

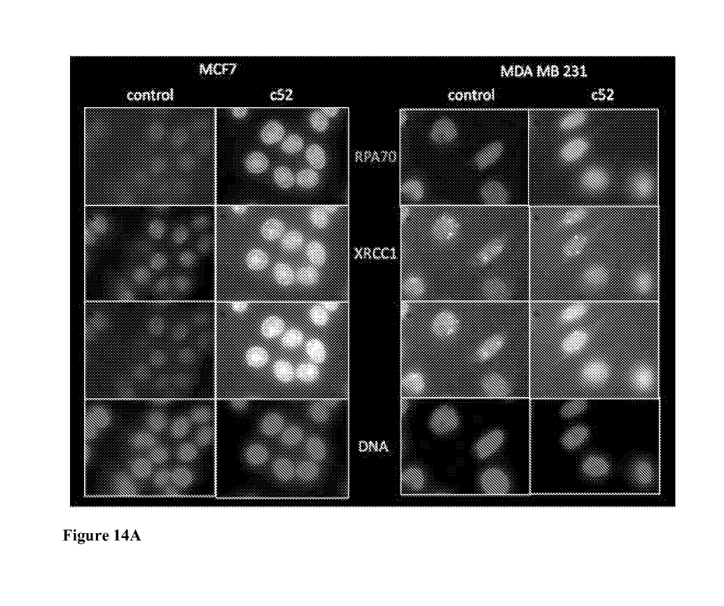

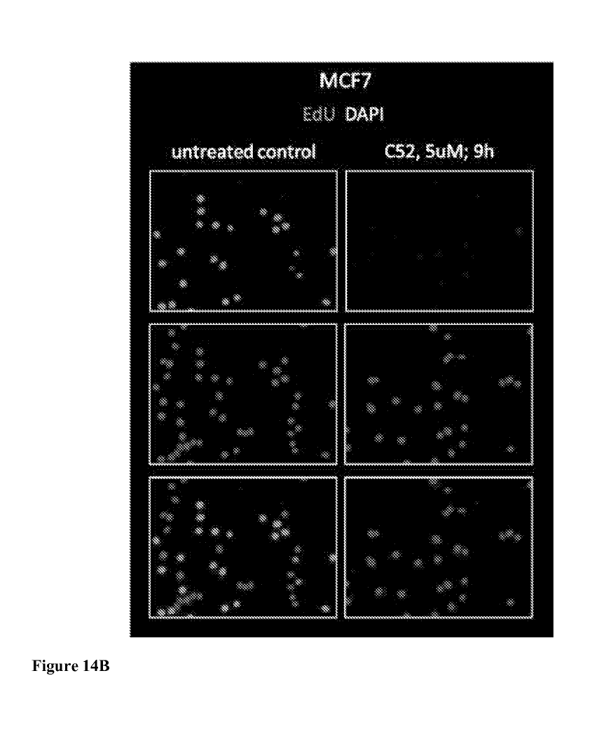

It was also found that c52 induces DNA-damage and p53 activation in sensitive, but not resistant cells (FIG. 13). Analysis of the type of DNA damage showed that it results from replication stress which c52 induces in sensitive, but not resistant cells (FIG. 14).

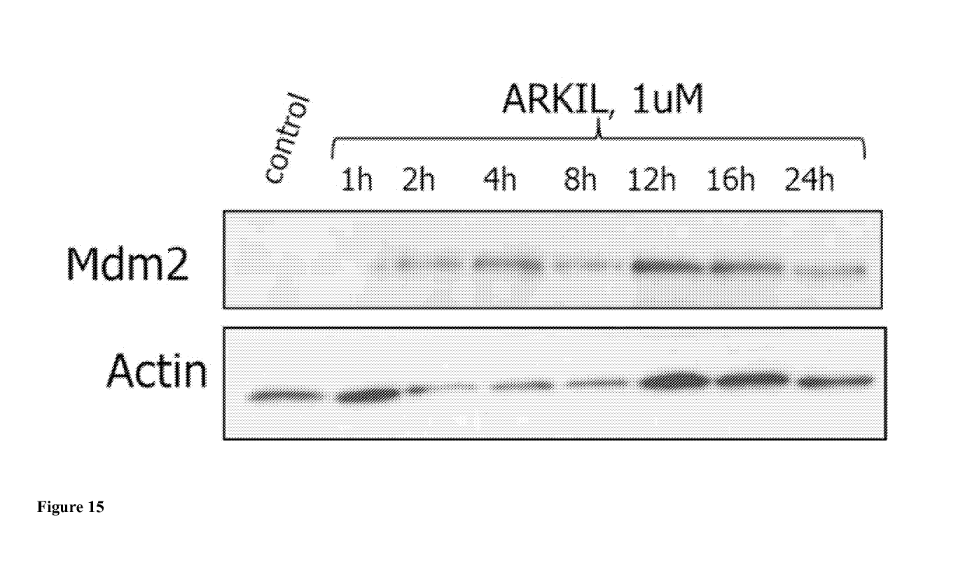

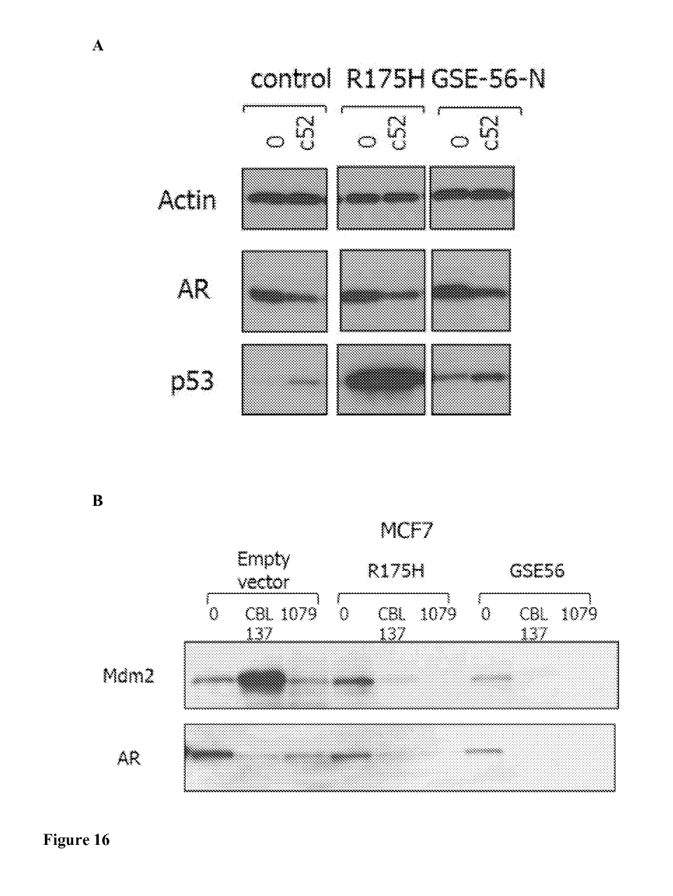

Also tested was the hypothesis that degradation of androgen receptor in prostate and breast cancer cells may be due to p53 induced accumulation of mdm2, which is also ubiquitin ligase for androgen receptor (FIG. 15). p53 was inactivated in several cell lines using different approaches what led to the blockade of mdm2 accumulation after c52 treatment. However androgen receptor was still degraded in c52 treated cells even in the absence of p53 activation and mdm2 accumulation (FIG. 16). It was concluded that c52 induces degradation of androgen receptor through p53 independent mechanism.

For target identification through affinity chromatography and sensitivity to proteolysis methods synthesis of c52 with flexible linker was carried out. PLA1098 demonstrated evidence of in vivo efficacy without any signs of toxicity.

### Example 32

This example provides compounds of the present disclosure and testing of the compounds against cancers.

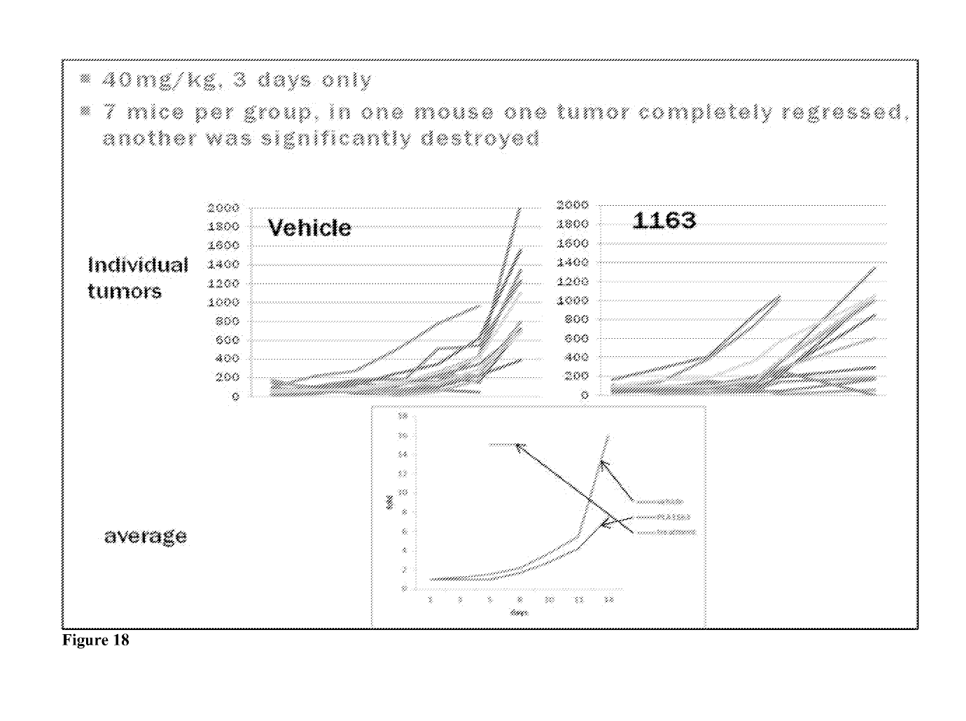

Pilot efficacy experiments were run for compounds: PLA1163, PLA1148 using the same model of subcutaneous xenografts of CWR22R cells in SCID mice (FIGS. 18 and 19).

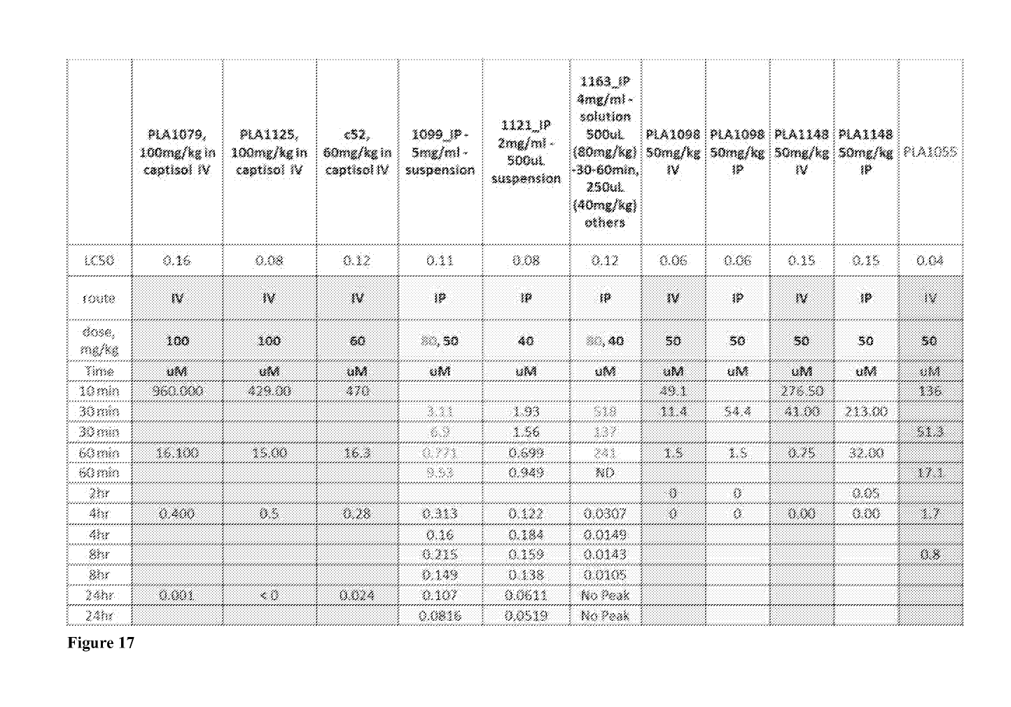

Summary of PK for tested compounds is shown in FIG. 17. Intra-tumor drug concentrations for all tested PLA compounds are shown in Table 2. LC50 data of in vitro 72 hours cytotoxicity assay are included in this table for comparison.

TABLE 2
Tissue drug concentrations for PLA compounds tested.

| | | | Tumor | | |
|---|---|---|---|---|---|
| | | Route of | concentration | LC50, | Liver |
| Compound | Dose | delivery | (nM) | nM | (nM) |
| c52 | 60 mg/kg | IP/IV | 21-24/0.5-4 nM | 150 | 23 |
| PLA1079 | 100 mg/kg | IP/IV | 24-48 nM | 120 | ND |
| PLA1125 | 100 mg/kg | IP/IV | 7-10 nM | 100 | ND |
| PLA1098 | 100 mg/kg | IP | 23-25 nM | 40 | 0 |
| PLA1148 | 50 mg/kg | IP | 0-90 nM | 200 | 50 |
| PLA1163 | 40 mg/kg | IP | 4-10 nM | 200 | 307 |

TABLE 3
PLA compounds selected for in vivo evaluation.

| | Reference No | State | Amount | series | Storage |
|---|---|---|---|---|---|
| PLA01055-00-01 | PLA01055 | Powder | 29.7 | c52 | RT |
| PLA01128-00-01 | PLA01128 | Powder | 39.5 | 219 | RT |
| Pla01164-00-02 | Pla01164-02 | Powder | 49.7 | 219 | RT |
| PLA01171-00-01 | PLA01171 | Powder | 23.1 | 219 | RT |
| PLA01173-00-02 | PLA01173-02 | Powder | 58.1 | 219 | RT |

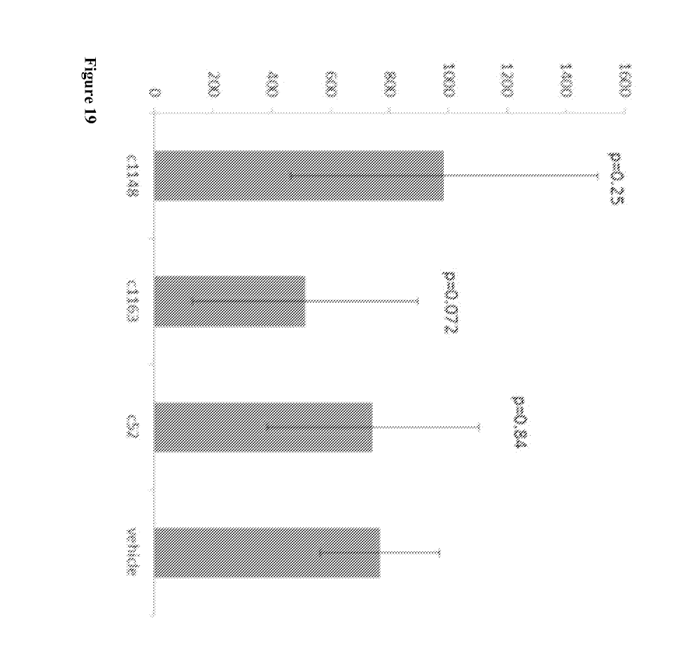

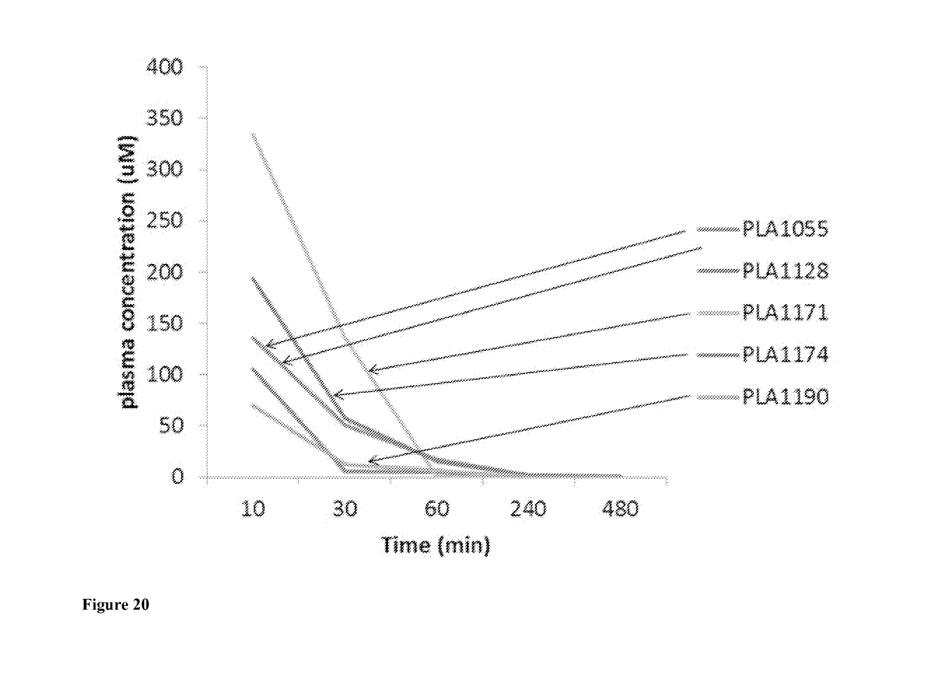

Formulations of the compounds PLA1055 and PLA128, PLA170, PLA171 and PLA1190 were developed. PK data for solubilized compounds are shown on FIG. 3. Compounds were tested for biological activity (Table 3) and 4 compounds were formulated for IV injections. Of the compounds tested for PK (FIG. 20), one showed low half-life. Another one, PLA1055, was detected up to 24 hours and at 8 hours at concentration slightly below 1 uM, the highest among the compounds tested. PLA1055 is also the most active compound in this group (LC50% 40 nM).

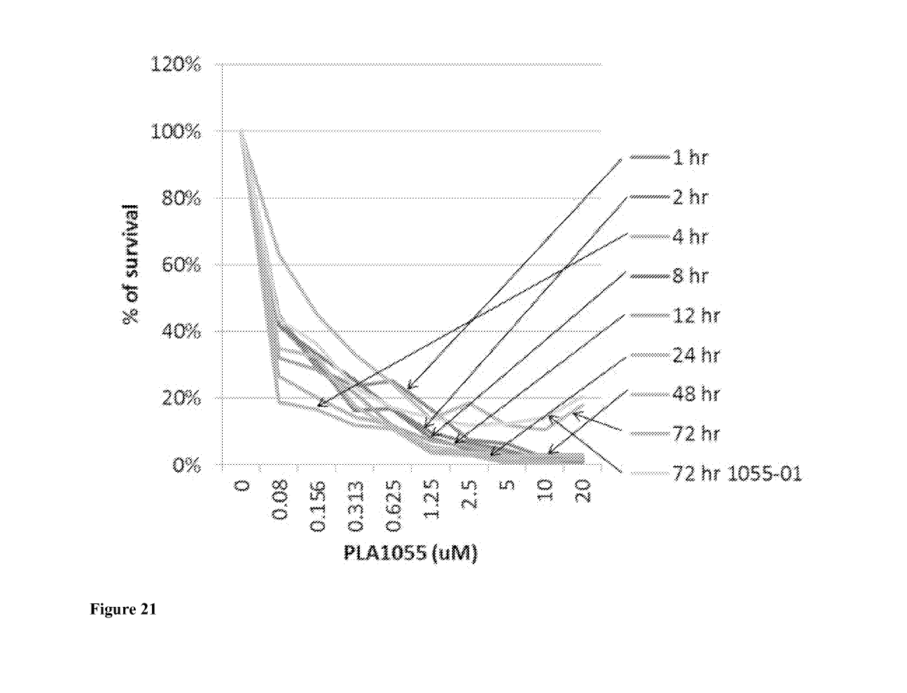

An in vitro experiment was run to detect the concentration of PLA1055 needed to kill all tumor cells in vitro (LC90) during different periods of time (30 minutes-72 hours, FIG. 21)). LC90 at 4-8 hours was 2.5 uM, what is similar to plasma concentrations at this point of time (FIG. 20).

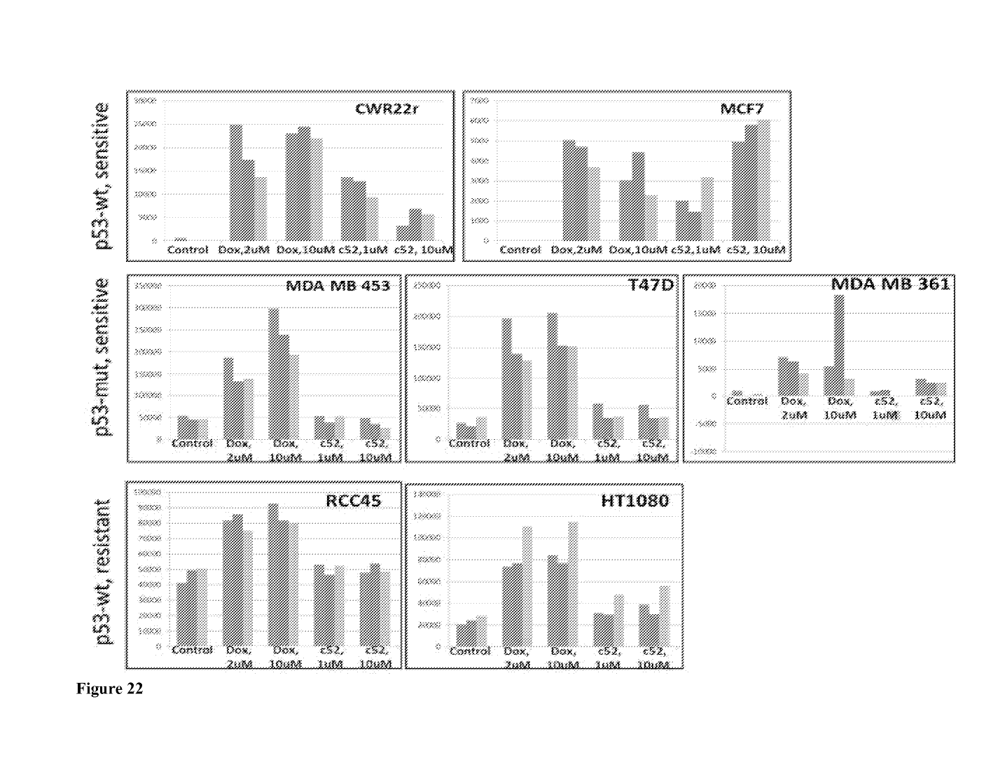

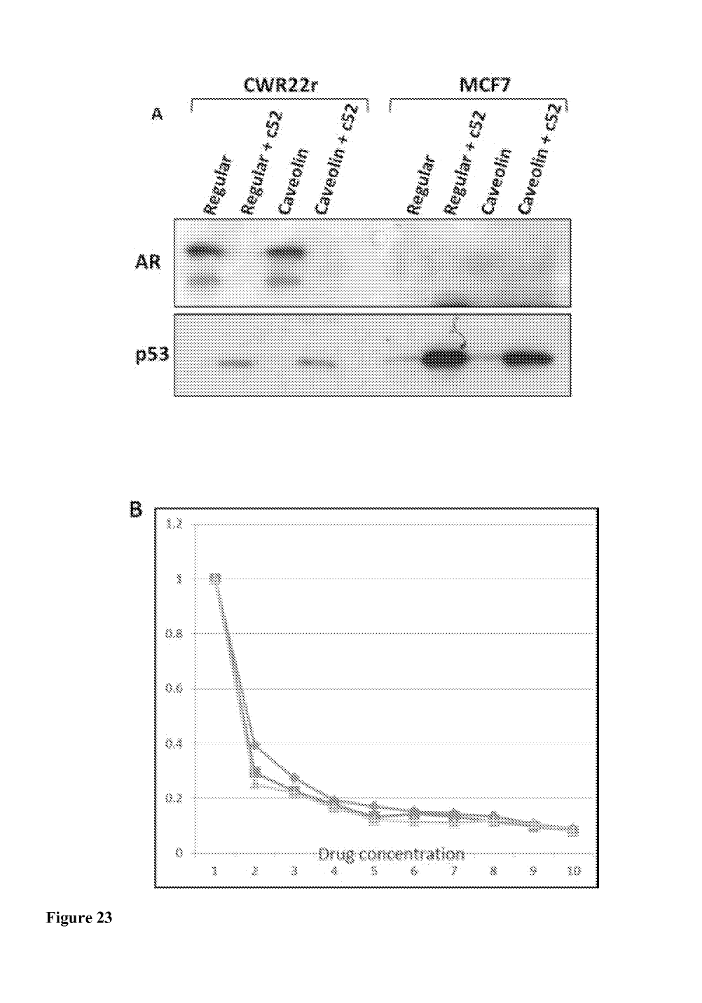

It was demonstrated that c52 induces apoptosis only in p53 wild type cells, although other sensitive cells die through non-apoptotic mechanism (FIG. 22). It was also found that replication stress happens in c52 treated cells only if they express AR. There are indications that AR may be important for replication in these cells. It was shown that Caveolinl-expressing cells were still were sensitive to c52, treatment with c52 still forced degradation of AR and activation of p53 (FIG. 23). Therefore, hypothesis of Caveolinl responsibility for c52 sensitivity was eliminated.

PLA1098 demonstrated evidence of in vivo efficacy without any signs of toxicity.

It was established that c52 causes replication stress in AR positive sensitive cells. This stress leads to activation of p53 and death of tumor cells through apoptosis mechanism. Death of cells without AR or with mutant p53 undergoes through alternative mechanism.

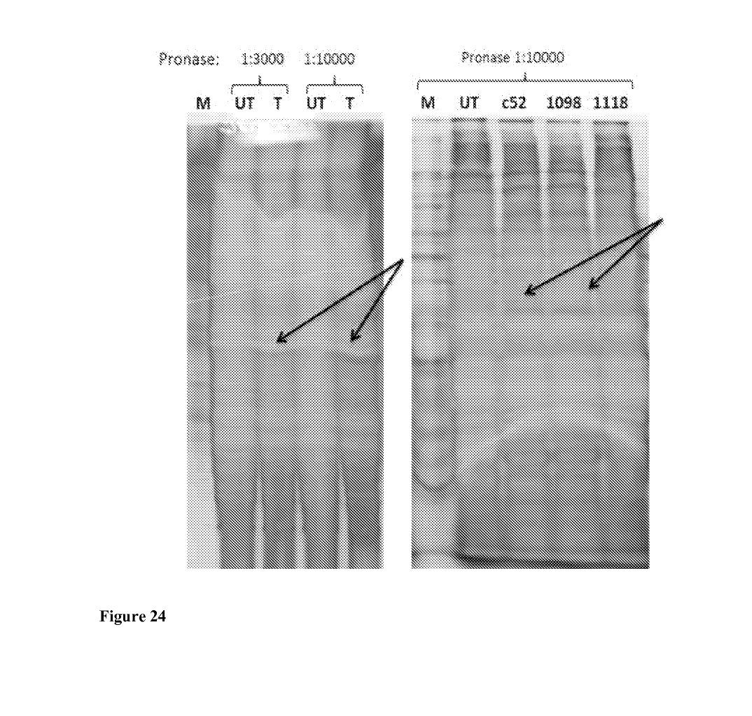

Target identification through affinity chromatography and sensitivity to proteolysis methods was undertaken. Experiments with different types of control were run, all of which demonstrated the presence of several bands resistant to proteolysis in lysates incubated either with c52 or with another active, but not with inactive compound. These bands were excised from gel and sent for protein sequencing. (FIG. 24).

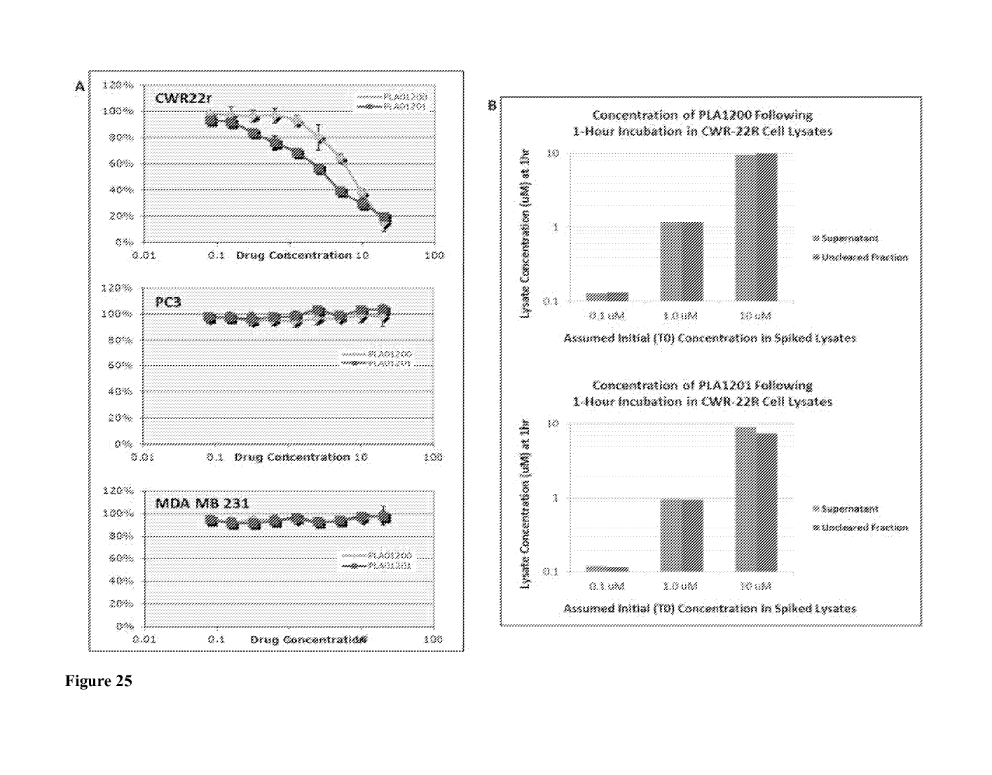

Two c52-like compounds with flexible linkers were synthesized. These compounds were tested for the presence of biological activity and stability in cell lysates. Both properties were confirmed. Biotin was attached to the flexible linker of one of the compounds to do affinity purification (FIG. 25).

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

What is claimed is:

1. A compound having the following structure: 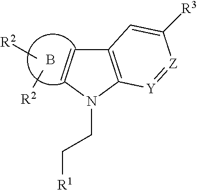
wherein R¹ is selected from the group consisting of H, CH₃, CH₂F, CHF₂, and CF₃; Y and Z are each independently are a nitrogen atom or carbon atom; ring B is a unsubstituted or substituted aryl or heteroaryl ring substituted with 0 to 2 R² groups; each R² is independently a hydrogen atom, a halogen atom, an alkoxy group, a nitrile group, or an amide group; and R³ is a five membered or six membered unsubstituted or substituted heterocycle, ketone, or nitrile, with the proviso that the compound does not have the following structure: 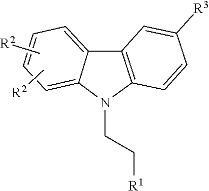
where R¹ is a hydrogen atom or CH₃, each R² is independently a hydrogen atom, an alkoxy group, a ketone, or a halide group and R³ is a ketone group.

2. The compound of claim 1, wherein the compound has the following structure: 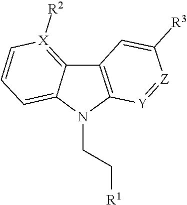
wherein when X is a carbon atom then R² is a hydrogen atom, a halogen atom, an alkoxy group, a nitrile group, or an amide group and when X is a nitrogen atom then R² is absent.

3. The compound of claim 1, wherein the compound has the following structure:

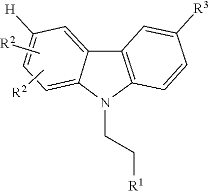

4. The compound of claim 1, wherein R¹ is selected from the group consisting of CH₃, CH₂F, and CHF₂.

5. The compound of claim 1, wherein the compound has the following structure: 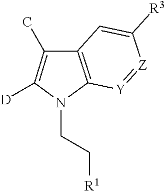
wherein C and D are replaced by the atoms of one the following structures: 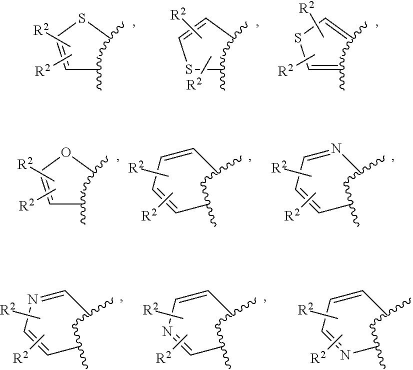
to form a ring.

6. The compound of claim 1, wherein R³ is selected from one of the following structures: 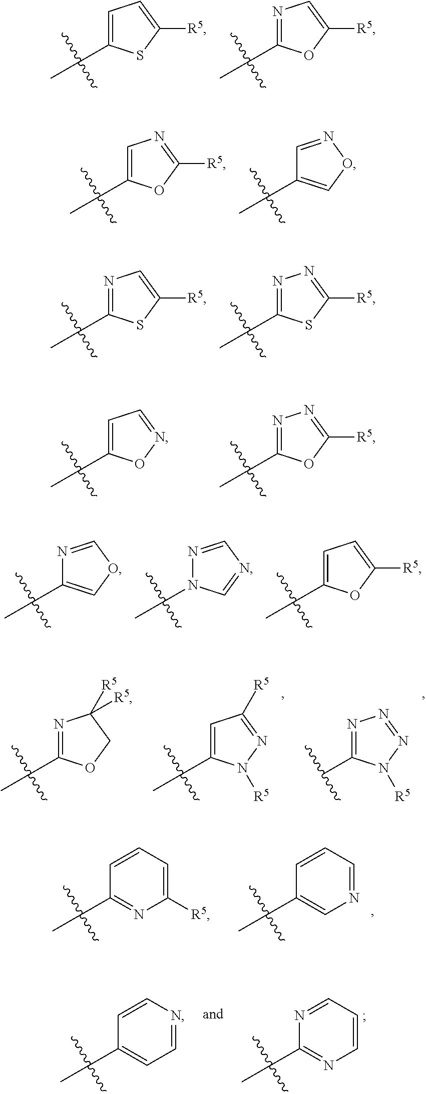
wherein each R⁵ is independently a hydrogen atom or alkyl group

7. The compound of claim 1, wherein the compound has the following structure:

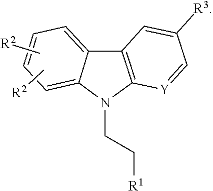

8. The compound of claim 1, wherein the compound has the following structure:

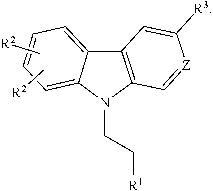

9. The compound of claim 1, wherein the compound has the following structure:

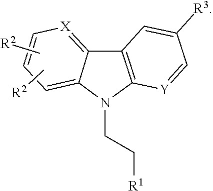

10. The compound of claim 1, wherein the compound has the following structure: 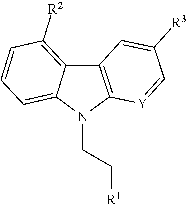
wherein R³ is a ketone or nitrile.

11. The compound of claim 1, wherein the compound has the following structure: 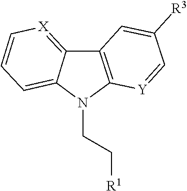
wherein R³ is a ketone or nitrile.

12. The compound of claim 1, wherein the compound has the following structure: 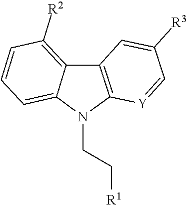
wherein R² is a fluorine atom, an alkoxy group, a nitrile group, or an amide group.

13. The compound of claim 1, wherein the compound has the following structure: 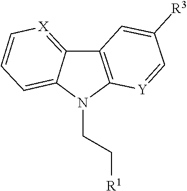
wherein R³ is a heterocyclic ring that only contains oxygen, sulfur, or a combination thereof.

14. The compound of claim 1, wherein the compound has the following structure: 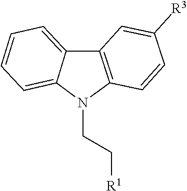
wherein R³ is a heterocyclic ring.

15. The compound of claim 1, wherein the compound has the following structure: 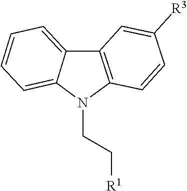
where R³ is a heteroaryl ring.

16. The compound of claim 1, wherein the compound has the following structure:

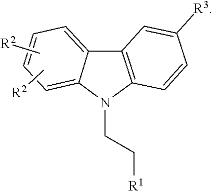

17. The compound of claim 1, wherein the compound is selected from the following structures:

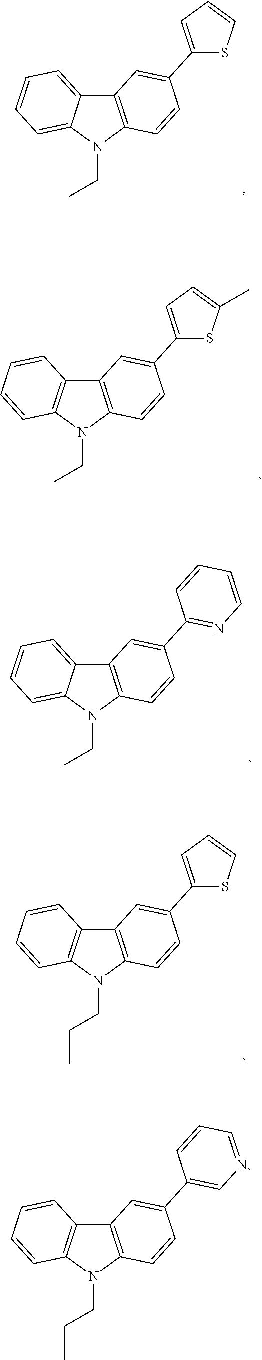 [SMILES: CCn1c2ccccc2c2cc(-c3cccs3)ccc21 ; CCn1c2ccccc2c2cc(-c3ccc(C)s3)ccc21 ; CCn1c2ccccc2c2cc(-c3ccccn3)ccc21 ; CCCn1c2ccccc2c2cc(-c3cccs3)ccc21 ; CCCn1c2ccccc2c2cc(-c3cccnc3)ccc21]

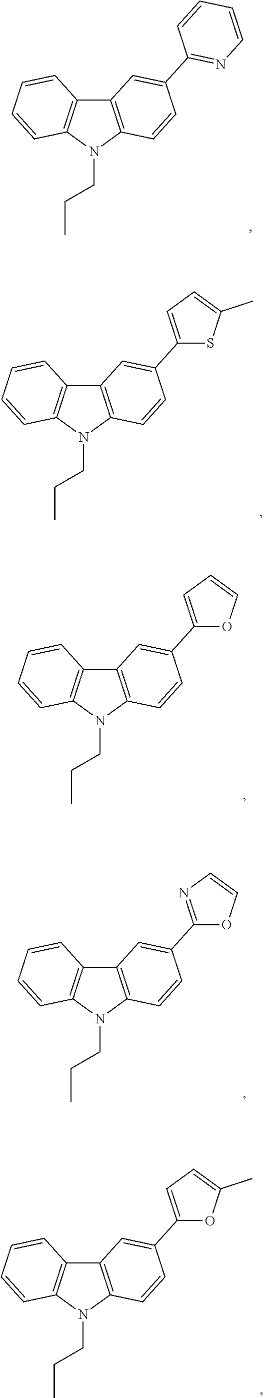 [SMILES: CCCn1c2ccccc2c2cc(-c3ccccn3)ccc21 ; CCCn1c2ccccc2c2cc(-c3ccc(C)s3)ccc21 ; CCCn1c2ccccc2c2cc(-c3ccco3)ccc21 ; CCCn1c2ccccc2c2cc(-c3ncco3)ccc21 ; CCCn1c2ccccc2c2cc(-c3ccc(C)o3)ccc21]

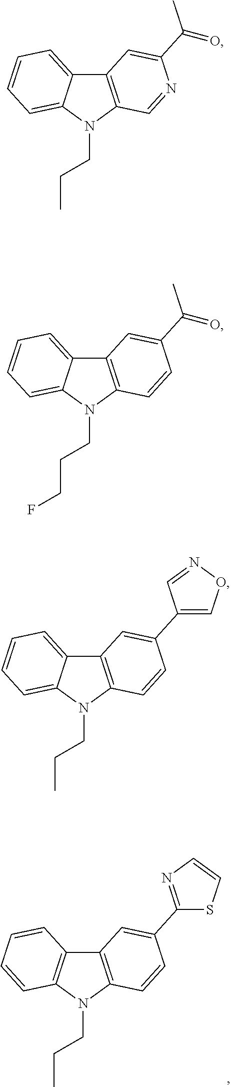 [SMILES: CCCn1c2ccccc2c2cc(C(C)=O)ncc21 ; CC(=O)c1ccc2c(c1)c1ccccc1n2CCCF ; CCCn1c2ccccc2c2cc(-c3cnoc3)ccc21 ; CCCn1c2ccccc2c2cc(-c3nccs3)ccc21]

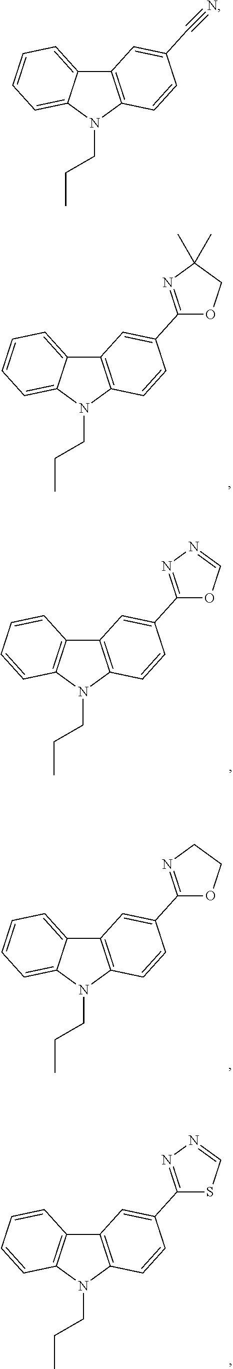 [SMILES: CCCn1c2ccccc2c2cc(C#N)ccc21 ; CCCn1c2ccccc2c2cc(C3=NC(C)(C)CO3)ccc21 ; CCCn1c2ccccc2c2cc(-c3nnco3)ccc21 ; CCCn1c2ccccc2c2cc(C3=NCCO3)ccc21 ; CCCn1c2ccccc2c2cc(-c3nncs3)ccc21]

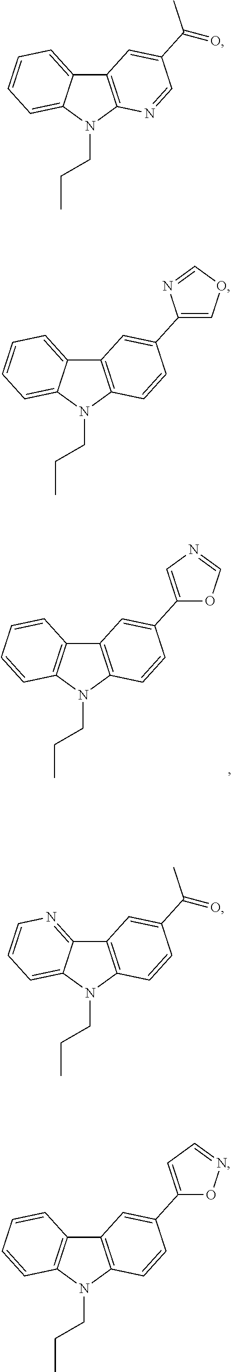 [SMILES: CCCn1c2ccccc2c2cc(C(C)=O)cnc21 ; CCCn1c2ccccc2c2cc(-c3cocn3)ccc21 ; CCCn1c2ccccc2c2cc(-c3cnco3)ccc21 ; CCCn1c2ccc(C(C)=O)cc2c2ncccc21 ; CCCn1c2ccccc2c2cc(-c3ccno3)ccc21]

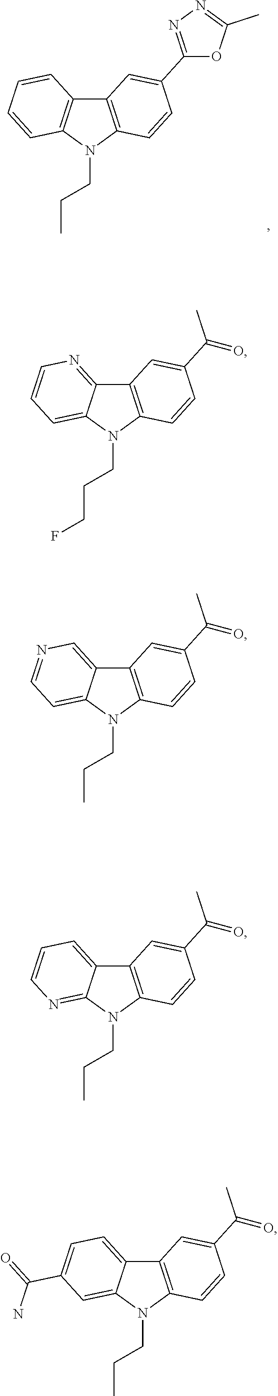 [SMILES: CCCn1c2ccccc2c2cc(-c3nnc(C)o3)ccc21 ; CC(=O)c1ccc2c(c1)c1ncccc1n2CCCF ; CCCn1c2ccncc2c2cc(C(C)=O)ccc21 ; CCCn1c2ccc(C(C)=O)cc2c2cccnc21 ; CCCn1c2ccc(C(C)=O)cc2c2ccc(C(N)=O)cc21]

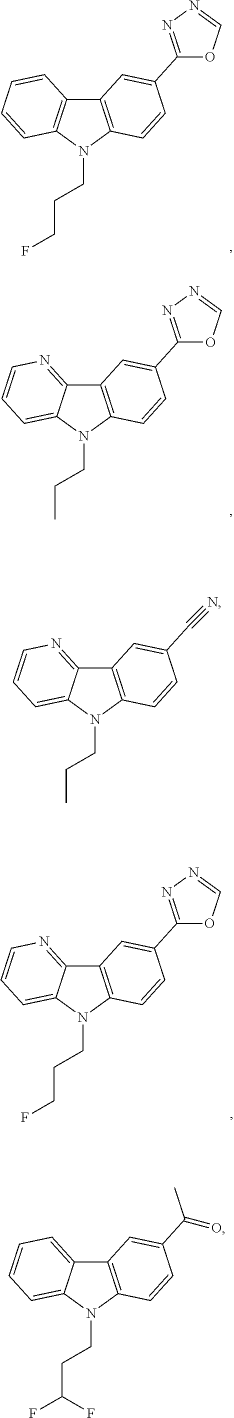 [SMILES: FCCCn1c2ccccc2c2cc(-c3nnco3)ccc21 ; CCCn1c2ccc(-c3nnco3)cc2c2ncccc21 ; CCCn1c2ccc(C#N)cc2c2ncccc21 ; FCCCn1c2ccc(-c3nnco3)cc2c2ncccc21 ; CC(=O)c1ccc2c(c1)c1ccccc1n2CCC(F)F]

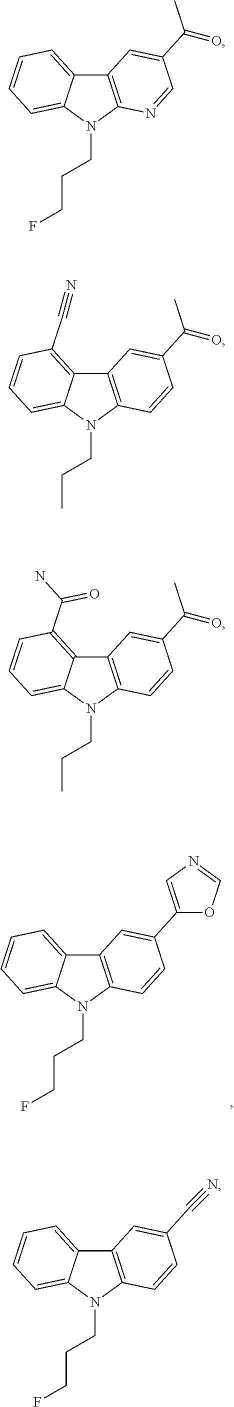 [SMILES: CC(=O)c1cnc2c(c1)c1ccccc1n2CCCF ; CCCn1c2ccc(C(C)=O)cc2c2c(C#N)cccc21 ; CCCn1c2ccc(C(C)=O)cc2c2c(C(N)=O)cccc21 ; FCCCn1c2ccccc2c2cc(-c3cnco3)ccc21 ; N#Cc1ccc2c(c1)c1ccccc1n2CCCF]

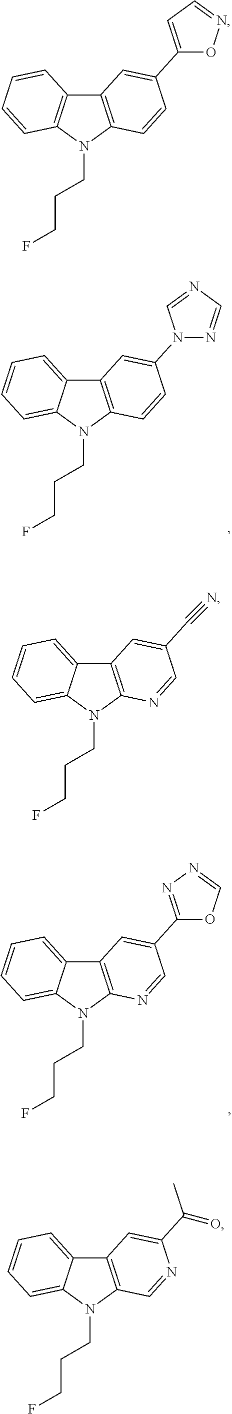 [SMILES: FCCCn1c2ccccc2c2cc(-c3ccno3)ccc21 ; FCCCn1c2ccccc2c2cc(-n3cncn3)ccc21 ; N#Cc1cnc2c(c1)c1ccccc1n2CCCF ; FCCCn1c2ccccc2c2cc(-c3nnco3)cnc21 ; CC(=O)c1cc2c3ccccc3n(CCCF)c2cn1]

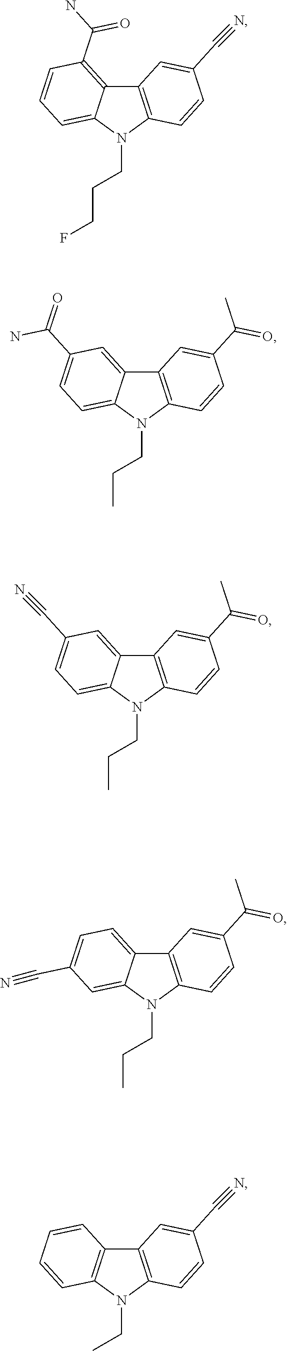 [SMILES: N#Cc1ccc2c(c1)c1c(C(N)=O)cccc1n2CCCF ; CCCn1c2ccc(C(C)=O)cc2c2cc(C(N)=O)ccc21 ; CCCn1c2ccc(C#N)cc2c2cc(C(C)=O)ccc21 ; CCCn1c2ccc(C(C)=O)cc2c2ccc(C#N)cc21 ; CCn1c2ccccc2c2cc(C#N)ccc21]

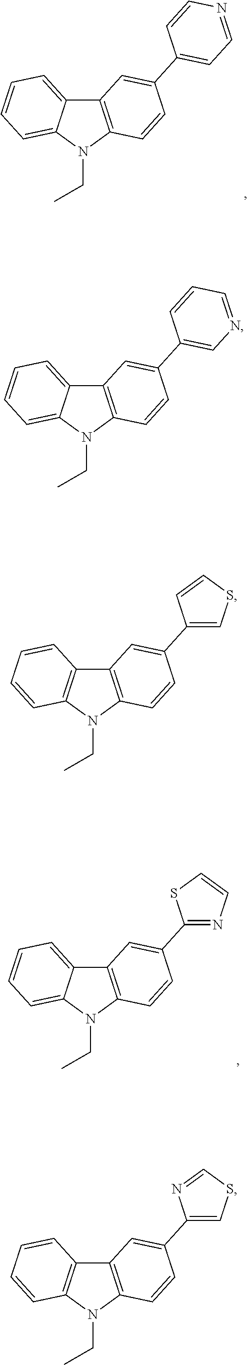 [SMILES: CCn1c2ccccc2c2cc(-c3ccncc3)ccc21 ; CCn1c2ccccc2c2cc(-c3cccnc3)ccc21 ; CCn1c2ccccc2c2cc(-c3ccsc3)ccc21 ; CCn1c2ccccc2c2cc(-c3nccs3)ccc21 ; CCn1c2ccccc2c2cc(-c3cscn3)ccc21]

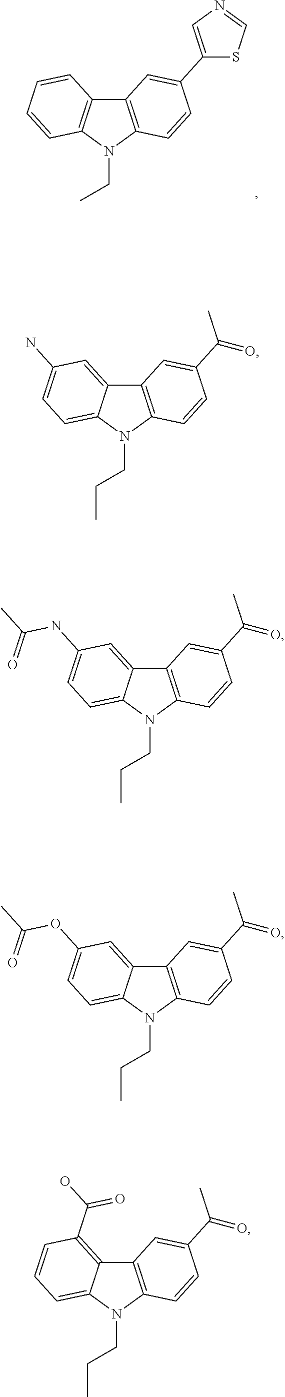 [SMILES: CCn1c2ccccc2c2cc(-c3cncs3)ccc21 ; CCCn1c2ccc(N)cc2c2cc(C(C)=O)ccc21 ; CCCn1c2ccc(NC(C)=O)cc2c2cc(C(C)=O)ccc21 ; CCCn1c2ccc(OC(C)=O)cc2c2cc(C(C)=O)ccc21 ; CCCn1c2ccc(C(C)=O)cc2c2c(C(=O)O)cccc21]

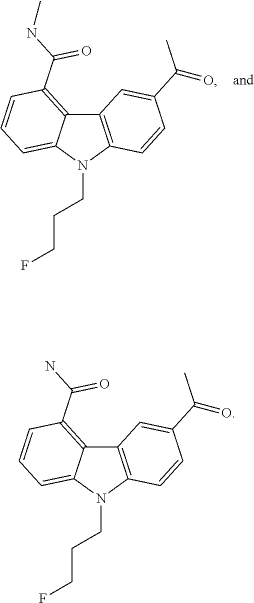 [SMILES: CNC(=O)c1cccc2c1c1cc(C(C)=O)ccc1n2CCCF ; CC(=O)c1ccc2c(c1)c1c(C(N)=O)cccc1n2CCCF]

18. A method for inhibiting the growth of AR positive or negative cancer cells in an individual diagnosed with or suspected of having AR positive or negative cancer comprising administering to the individual a composition comprising a compound of claim 1.
